(19) United States
(12) Patent Application Publication
(10) Pub. No.: US 2009/0202515 A1
(43) Pub. Date: Aug. 13, 2009

(54) AGENTS FOR SUPPRESSING NEURAL FIBROTIC DEGENERATION

(75) Inventors: Hiroyuki Yoneyama, Tokyo (JP); Kenji Ishida, Tokyo (JP); Jun Koyama, Tokyo (JP)

Correspondence Address: BOZICEVIC, FIELD & FRANCIS LLP, 1900 UNIVERSITY AVENUE, SUITE 200, EAST PALO ALTO, CA, 94303, US

(73) Assignee: Stelic Institute of Regenerative Medicine, Stelic Institute & Co.

(21) Appl. No.: 11/576,043

(22) PCT Filed: Nov. 28, 2006
(86) PCT No.: PCT/JP2006/323678

§ 371 Date: Feb. 22, 2008

(30) Foreign Application Priority Data
Sep. 8, 2006 (JP) 2006-244532

(51) Int. Cl. A61K 38/47; C12N 9/24; C12Q 1/34

(52) U.S. Cl. 424/94.61; 435/200; 435/18

(57) ABSTRACT

The present invention examined the accumulation of chondroitin sulfate proteoglycans (CSPGs). The present invention relates to neurodegeneration-suppressing agents that are suitable for gene therapy or prevention of neural fibrotic degenerative diseases which induce neural cell death due to an accumulation of abnormal proteins, where the therapies are based on siRNAs against N-acetylgalactosamine-4-O-sulfotransferases (N-acetylgalactosamine-4-O-sulfotransferase-1, N-acetylgalactosamine-4-O-sulfotransferase-2, and N-acetylgalactosamine-4-sulfate 6-O-sulfotransferase (GalNAc4ST-1, GalNAc4ST-2, and GALNAC4S-6ST, respectively)), which are sulfotransferases for acetylgalactosamine, a CSPG side chain, and chondroitinase ABC, an enzyme that degrades chondroitin sulfate, another CSPG side chain.

### TECHNICAL FIELD

The present invention relates to therapeutic and preventive agents for neural fibrotic degenerative diseases and methods for suppressing neural fibrotic degeneration, wherein the methods are based on controlling the accumulation of chondroitin sulfate proteoglycans (CSPG), and also relates to therapeutic or preventive methods for neural fibrotic degenerative diseases including Alzheimer's disease (AD), Parkinson's disease (PD), amyotrophic lateral sclerosis (ALS), and the like, based on the methods described above.

### BACKGROUND ART

Of the neural fibrotic degenerative diseases, cerebral neural fibrotic degenerative diseases are considered intractable diseases caused by a decrease in neurons resulting from neuronal cell death. Neural fibrotic degenerative diseases are roughly divided into two groups: one group develops memory-related and dementia-related symptoms, and the other group develops movement-related symptoms. Representative examples include Alzheimer's disease in the former group, and Parkinson's disease in the latter group. There are more than 18 million Alzheimer's patients in the world, and the number is predicted to increase to 34 million in the next 20 years (Alzheimer's Disease International (ADI)). In Japan, there are currently at least 300 000 patients, and the number is predicted to be over 800,000 after four years, in 2010. Genetic analysis and statistics show that the aged frequently develop the disease, which is more frequent in females than in males (according to a survey by Marumiya Wakanyaku Kenkyujo (Marumiya Oriental Drug Institute).

There are two types of Alzheimer's disease: familial Alzheimer's disease which occurs genetically, and sporadic Alzheimer's disease, which is caused by abnormalities in metabolic balance (reduced acetylcholine levels) resulting from a malfunction of factors involved in the production and degradation of amyloid β peptide (Aβ). Both types are primarily caused by an accumulation of Aβ in the brain, and manifest as the appearance of senile plaques in nerve cells. Aβ is formed by cleavage from the amyloid precursor protein (APP) by protease, and is reported to become insoluble and aggregate upon an increase in the proportion of β-sheet conformation. A recent notable discovery was the identification of neprilysin, a degradative enzyme that prevents amyloid accumulation; a reduction in the activity of this enzyme has been shown to increase Aβ accumulation in the brain. In conjunction with this is a report conforming a reduction in brain Aβ level upon expression of neprilysin in the brain using adenoviruses (Non-Patent Document 1). Furthermore, a gene expressing apolipoprotein (apo E) has been reported as a risk factor for Alzheimer's disease and is closely associated with the disease (Non-Patent Document 2). In addition to these findings, intensive studies of various factors have revealed a number of causative proteins, gene mutations, and the like. For example, there are reports that the accumulation of highly phosphorylated tau protein causes neurofibrillary degeneration, and that mutations in the genes encoding the amyloid precursor protein (APP) and presenilin 1 and 2 accelerate the accumulation of Aβ (Non-Patent Document 3). As described above, many studies have been reported; however, the only therapeutic agent for Alzheimer's disease approved for sale in Japan is donepezil hydrochloride (brand name: Aricept), which for symptoms of Alzheimer's disease has the effect of slowing dementia progression by about nine months, but not of promoting the restoration of cognitive function. This therapeutic agent is used to increase reduced acetylcholine levels in the brain by inhibiting acetylcholine esterase, an enzyme that degrades acetylcholine, which is a neurotransmitter involved in memory and learning. Donepezil hydrochloride does not serve as an eradicative drug for Alzheimer's disease and is merely a drug administered only to mild patients. Currently, various research institutes are creating animal models to discover factors involved in the onset of Alzheimer's disease. In spite of trial and error, neither decisive therapeutic methods nor agents have been established because many factors are involved in the disease.

Another central neural fibrotic degenerative disease listed as intractable is Parkinson's disease, for which the number of patients is less than Alzheimer's disease. Parkinson's disease is caused when the amount of dopamine (a neurotransmitter produced in neurons in a part of the mid brain known as the substantia nigra) is reduced by the death of dopamine neurons in the substantia nigra, resulting in an imbalance between dopamine and another neurotransmitter, acetylcholine, and causing a relative increase in acetylcholine level. The four primary symptoms are: tremors (shaking), muscle rigidity, slowness of movement, and impaired postural control. In Japan the disease tends to afflict more females, with a male:female ratio of 1:1.5-2, and it afflicts a wide range of ages, from the 30s to 80s. Currently, the disease afflicts about 100 in every 100 000 people (Minutes of the Committee on Measures Against Intractable Diseases, Disease Control Section, Health Sciences Council of Japan), and the number of people with the disease is estimated to be about 120,000 in Japan. Hereditary Parkinson's disease accounts for 5-10% of all of patients. Currently, various countries all over the world are studying actively to find causes of the disease. Gene analysis of patients with familial Parkinson's disease has identified some causative genes: α-synuclein (abnormal folding and accumulation of the protein), parkin (protein degradation system and mitochondrial function), PINK1 (mitochondrial function), DJ-1 (oxidative stress), and LRRK2 (phosphorylation). However, it still remains unclear as to how these genes are involved in Parkinson's disease. Research has also been reported suggesting that a cause of neural fibrotic degenerative diseases is protein accumulation resulting from mutations in genes involved in the proteolysis mechanism of misfolded or aggregated proteins in the brain (Non-Patent Documents 4 to 8). In addition to the above, other recently reported neural fibrotic degenerative diseases include polyglutamine disease, amyotrophic lateral sclerosis, myelopathic muscular atrophy, Huntington's disease, and multiple sclerosis; endoplasmic reticulum stress due to the accumulation of abnormal proteins has also been reported to cause neural cell death in these diseases. Normally, abnormal proteins are degraded and removed by protein degradation systems (unfolded protein response (UPR), ER associated degradation (ERAD), and ubiquitin-proteasome). However, cells that cannot sufficiently respond to excess abnormal proteins induce cell death signals (Non-Patent Document 9). Thus, complicated signal transductions occur in cells and induce neural fibrotic degeneration via various pathways. For each disease, studies of signal transductions and such have been conducted; however, they are complicated and it is thus extremely difficult to develop therapeutic agents targeted at the signal transductions.

To date, therapeutic methods for Parkinson's disease are most advanced from among the various neural fibrotic degenerative diseases; the methods are divided into physical and exercise therapy, surgical therapy, and chemical therapy. Chemical therapy is most commonly used. In particular, agents most effective against tremors or other symptoms are used, such as L-DOPA (a dopamine precursor), dopamine receptor stimulants (potentiaters of the L-DOPA effect), anticholinergic drugs (acetylcholine suppressors), dopamine release-stimulating agents, noradrenaline supplements (drugs against the “freezing phenomenon”), inhibitors of dopamine degradation (MAO-B inhibitors and COMT inhibitors), and such, depending on symptoms. However, these therapeutic agents do not work as curative medicines and are used as medicines to relieve symptoms. Long-term administration of the agents has also been confirmed to cause various problems. For example, long term administration of L-DOPA over five to ten years results in a “wearing-off” phenomenon (a shortened period of effectiveness), an “on-off” phenomenon (loss of effectiveness), dyskinesia (involuntary movement), and neurological manifestations such as hallucinations and delusions. As society continues to age, there is demand for the development of new drugs that take a completely different approach, namely, drug therapies that not only temporarily relieve symptoms, but that can also very safely and effectively inhibit the disease or suppress side effects using a mechanism that probes for and inhibits underlying causes of the disease. Central research institutes across the world are currently at the forefront of progressing studies into neurological diseases of the brain. Various drug discoveries have been devised, and currently “brain gene therapy” is increasingly expected to be one fundamental therapy (Non-Patent Documents 10 to 12).

Herein the focus is on proteoglycans for therapies for the above neural fibrotic degenerative diseases; proteoglycans are molecules structured such that one or more glycosaminoglycan (GAG) chains are covalently linked with proteins called “core proteins”, and the specific sugar chain structures in GAG chains are thought to be involved in various proteoglycan functions. Proteoglycans are divided into four groups, based on the type of GAG chain: chondroitin sulfate proteoglycans (CSPGs), dermatan sulfate proteoglycans (DSPGs), heparan sulfate proteoglycans (HSPGs), and keratan sulfate proteoglycans (KSPGs) (Non-Patent Documents 13 to 19). Of these, HSPGs have been studied extensively, since their functions are greatly modified by binding to various cytokines, adhesion molecules, and chemokines. HSPGs and GAG chains, which are side chains extending from the core proteins, are reported to convert nonfibrillar amyloid β-proteins into neurotoxic fibrillar amyloid β-proteins, and to protect amyloid β-proteins (Aβ) from the protein degradation system (Non-Patent Documents 20 to 22). Likewise, heparan sulfate proteoglycans and glycosaminoglycans are suspected to convert α-synuclein, the most likely candidate for the causative agent of Parkinson's disease, in to its fibrillar form, and also to denature amyloid precursor proteins (APPs), which are causative proteins of Alzheimer's disease (Non-Patent Document 23).

Meanwhile, CSPGs are essential molecules during the fetal period, and are abundant in each organ. CSPGs are thought to be molecules that regulate processes involved in nerve regeneration, such as inducers of differentiation for neural stem cells (Patent Document 1), and molecules involved in nerve regeneration using human/osteogenic proteins (Patent Document 2) or human bone morphogenetic proteins (Patent Document 3); CSPGs are also involved in the inhibition of nerve regeneration at various sites, as described in “Treatment of Damages in the Central Nervous System” (Patent Document 4), “Materials and Methods for Promoting the Repair of Nerve Tissues” (Patent Document 5), “Inhibitory Factors in Treating Vascular Smooth Muscle Cells” (Patent Document 6), and such. For example, the chondroitin sulfate proteoglycans “neuron”, “brevican”, and “NG2” were first identified as regeneration inhibitory substances expressed upon central nerve damage (Non-Patent Document 24); and there is a report that the administration of chondroitinase ABC (an enzyme that selectively removes chondroitin sulfate, a type of GAG chain) will degrade these CSPGs and promote central nerve regeneration (Patent Document 4). When these CSPGs are expressed they are reported to accumulate in the Lewy body (LB) of the brain in Parkinson's disease patients; however, the biological function of this remains unclear (Non-Patent Document 25). The significance of increased CSPGs in the brain also remains unknown (Non-Patent Document 23).

[Patent Document 1] Japanese Patent Application Kokai Publication No. (JP-A) 2005-278641 (unexamined, published Japanese patent application)

### [Patent Document 2] JP-A2005-007196

[Patent Document 3] Japanese Patent Kohyo Publication No. (JP-A) H09-501932 (unexamined Japanese national phase publication corresponding to a non-Japanese international publication)

### [Patent Document 4] JP-A 2005-526740

### [Patent Document 5] JP-A 2005-500375

### [Patent Document 6] JP-A H08-510209

### [Non-Patent Document 1] Iwata et al., J. Neurosci. (2004) 24(4) 991-998

[Non-Patent Document 2] Strittmatter, W. J et al., Proc. Natl. Acad. Sci. (1993) 90: 8098-8102
[Non-Patent Document 3] Forman et al. Nat. Med. (2004) 10(10): 1055-1063
[Non-Patent Document 4] Matthew, J. F. Nature. review (2006) 7: 306-318
[Non-Patent Document 5] Gsponer, J. et al. Protein Pept. Lett. (2006) 13(3): 287-293
[Non-Patent Document 6] McNaught, K. S. P. et al., Neurobiol. Aging (2006) 27: 530-545

### [Non-Patent Document 7] Hattori, N. et al., Lancet (2004) 364(9435): 722-724

[Non-Patent Document 8] Murakami, T. et al., Ann. neurol. (2004) 55(3): 439-442

### [Non-Patent Document 9] Kadowaki, H. et al., Jikken Igaku (Experimental Medicine) (2006) 24: 10173-180

### [Non-Patent Document 10] Muramatsu, S. et al., Rinsho Shinkeigaku. (2005) 45 (11): 902-904

[Non-Patent Document 11] Iwata. N. et al., J. Neurosci. (2004) 24(4): 991-998
[Non-Patent Document 12] Hadaczek P, et al., Hum. Gene Ther. (2006) 17(3): 291-302

### [Non-Patent Document 13] Lindahl, U et al., (1972) In Glycoproteins (Gottschalk, A. ed) pp. 491-517, Elsevier, New York.

[Non-Patent Document 14] Oegema, T et al., J. Biol. Chem. (1984) 259: 1720-1726
[Non-Patent Document 15] Sugahara, K et al., J. Biol. Chem. (1988) 263: 10168-10174
[Non-Patent Document 16] Sugahara, K et al., J. Biol. Chem. (1992) 267: 6027-6035
[Non-Patent Document 17] De Waard et al., J. Biol. Chem. (1992) 267: 6036-6043
[Non-Patent Document 18] Moses, J, Oldberg et al., Eur. J. Biol. (1992) 248: 521-526

### [Non-Patent Document 19] Yamada, S et al., Trends in Glycoscience and Glycotechnology, (1998) 10: 95-123

### [Non-Patent Document 20] Castillo, G. M. et al., J. Neurochem. (1997) 69: 2452-2465

[Non-Patent Document 21] Cotman, S. L. et al., Mol. Cell. Neurosci. 15 (2000): 183-198
[Non-Patent Document 22] Snow, A. D. et al., Neurobiol. Aging (1989)10: 481-497

### [Non-Patent Document 23] Horsen, J. V. et al., J. Alzheimers Dis. (2004)6: 469-474

### [Non-Patent Document 24] Rhodes K E, Fawcett J W, J. Anat. (2004) 204: 33-48

### [Non-Patent Document 25] DeWitt, D. A. et al., Brain Res. (1994) 656: 205-209

### DISCLOSURE OF THE INVENTION

### Problems to Be Solved by the Invention

An objective of the present invention is to provide neural fibrotic degeneration-suppressing agents, therapeutic agents against neural fibrotic degenerative diseases that comprise the above agents as active ingredients, and methods of screening for neural fibrotic degeneration-suppressing agents.

As the present inventors continued their dedicated studies into developing such agents, they hypothesized that the excess accumulation of CSPGs, which had not yet been recognized as a pathogenic agent of neural fibrotic degenerative diseases, not only stimulated neuronal death and synaptic defects (Alzheimer's disease), death of dopamine neurons (Parkinson's disease), and motor neuron degeneration (amyotrophic lateral sclerosis), but also affected various factors of neural fibrotic degenerative diseases. Based on this hypothesis, the present inventors conducted studies using a mouse model for Parkinson's disease, one of the neural fibrotic degenerative diseases. As a result, the inventors found that siRNA-mediated knockdown of genes encoding GalNAc4ST-1, GalNAc4ST-2, and GALNAC4S-6ST, which are CSPG sulfotransferases, or treatment with chondroitinase ABC, which removes sugar chains from chondroitin sulfate, suppressed the overexpression or overaccumulation of CSPGs in dopamine neurons, and thus activated the regulation of nerve cell function by glial cells called astrocytes. The inventors also found that these treatments suppressed the infiltration of fibrosing cells, prevented death of dopamine neurons, enhanced the secretion of tyrosine hydroxylase (TH), which is a dopamine synthetase, and thus these treatments were discovered to be therapeutic for symptoms of Parkinson's disease. Specifically, the inventors demonstrated that neural fibrotic degeneration could be suppressed by inhibiting the biosynthesis or accumulation of chondroitin sulfate proteoglycans, and they thus completed the present invention. Substances that inhibit the production or accumulation of chondroitin sulfate proteoglycans are useful as neural fibrotic degeneration-suppressing agents. In addition, the agents can also be used as therapeutic or preventive agents for neural fibrotic degenerative diseases.

There are no reports of actual clinical treatment using the above-described chondroitinase ABC, which degrades chondroitin sulfate, nor of siRNAs, which can conceivably knockdown sulfotransferases, to control the accumulation of proteoglycans, nor to observe changes in the pathological conditions of cerebral neural fibrotic degenerative diseases. An example is shown herein that focuses on administering chondroitinase ABC, a chondroitin sulfate-degrading enzyme, and siRNAs against N-acetylgalactosamine-4-O-sulfotransferases (N-acetylgalactosamine-4-O-sulfotransferase-1 (GalNAc4ST-1), N-acetylgalactosamine-4-O-sulfotransferase-2 (GalNAc4ST-2), and N-acetylgactosamine-4-sulfate 6-O-sulfotransferase (GALNAC4S-6ST)), which are sulfotransferases that act on glycosaminoglycans, which are helical side chains extending from core proteins.

The present invention relates to neural fibrotic degeneration-suppressing agents, therapeutic agents for neural fibrotic degenerative diseases that comprise the above agents as an active ingredient, and methods of screening for neural fibrotic degeneration-suppressing agents. More specifically, the present invention provides:

[1] a neural fibrotic degeneration-suppressing agent comprising as an active ingredient a substance that inhibits the production or accumulation of a chondroitin sulfate proteoglycan;
[2] the agent of [1], wherein the substance has an activity of promoting the degradation of a chondroitin sulfate proteoglycan;
[3] the agent of [1], wherein the substance has an activity of inhibiting the synthesis of a chondroitin sulfate proteoglycan;
[4] the agent of [1], wherein the substance has an activity of desulfating a chondroitin sulfate proteoglycan;
[5] the agent of [1], wherein the substance has an activity of inhibiting the sulfation of a chondroitin sulfate proteoglycan;
[6] the agent of any of [1] to [5], wherein the production or accumulation of a chondroitin sulfate proteoglycan is inhibited in a brain;
[7] the agent of any of [1] to [6], which is used for treating or preventing a neural fibrotic degenerative disease;
[8] the agent of [7], wherein the neural fibrotic degenerative disease is a cerebrospinal or peripheral neural fibrotic degenerative disease;
[9] the agent of [7], wherein the neural fibrotic degenerative disease is Parkinson's disease, Alzheimer's disease, amyotrophic lateral sclerosis, polyglutamine disease, myelopathic muscular atrophy, Huntington's disease, or multiple sclerosis;
[10] a method of screening for a neural fibrotic degeneration-suppressing agent, which comprises selecting from a test sample a substance with an activity of inhibiting the production or accumulation of a chondroitin sulfate proteoglycan;
[11] the method of [10], which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan; and
[12] the method of [10] or [11], wherein the neural fibrotic degeneration-suppressing agent is used for treating or preventing a neural fibrotic degenerative disease.

Furthermore, the present invention relates to:

[13] use of the agent of any of [1] to [9] in the preparation of a neural fibrotic degeneration-suppressing agent;
[14] a method for treating a neural fibrotic degenerative disease, comprising the step of administering the agent of any of [1] to [9] to a subject (patient, etc.); and
[15] a composition comprising the agent of any of [1] to [9] and a pharmaceutically acceptable carrier.

### EFFECT OF THE INVENTION

The present invention has demonstrated that the production and accumulation of chondroitin sulfate proteoglycans is involved in the onset of neural fibrotic degeneration. Inhibiting the production and accumulation of chondroitin sulfate proteoglycans was shown to suppress neural fibrotic degeneration. Thus, therapeutic agents for neural fibrotic degenerative diseases can be provided based on this new concept. In particular, neural fibrotic degeneration is closely associated with Parkinson's disease, Alzheimer's disease, and the like, patient numbers of which are increasing in today's society. Therefore, such therapeutic agents based on this new concept have great medical and industrial significance.

### BRIEF DESCRIPTION OF THE DRAWINGS

FIG. 1 is a set of photographs showing RT-PCR evaluation of the expression of β-actin, GalNAc4ST-1, and GALNAC4S-6ST on Day 8 (the last day) in the untreated group, the chondroitinase ABC-treated group, and the GalNAcST siRNA-treated group of Parkinson's disease model mice induced with 1-methyl-4-phenyl-1,2,3,6-tetrahydropyridine (MPTP).

FIG. 2 is a set of photographs showing intracerebral deposition of chondroitin sulfate proteoglycans (CSPGs) in the untreated group, the GalNAcST siRNA-treated group, and the chondroitinase ABC-treated group of Parkinson's disease model mice induced with MPTP.

FIG. 3 is a set of photographs showing the infiltration of F4/80-positive inflammatory macrophages in the untreated group, the GalNAcST siRNA-treated group, and the chondroitinase ABC-treated group of Parkinson's disease model mice induced with MPTP.

FIG. 4 is a set of photographs showing intracerebral fibroblasts in the untreated group, the GalNAcST siRNA-treated group, and the chondroitinase ABC-treated group of Parkinson's disease model mice induced with MPTP.

FIG. 5 is a set of photographs showing astrocytes (glial cells) in the untreated group, the GalNAcST siRNA-treated group, and the chondroitinase ABC-treated group of Parkinson's disease model mice induced with MPTP.

FIG. 6 is a set of photographs showing tyrosine hydroxylase (TH) secretion in the untreated group, the GalNAcST siRNA-treated group, and the chondroitinase ABC-treated group of Parkinson's disease model mice induced with MPTP.

### BEST MODE FOR CARRYING OUT THE INVENTION

The present invention is specifically described below:

Pathological conditions associated with Parkinson's disease, a representative neural fibrotic degenerative disease, include cerebral nerve cell degeneration such as fibrosis due to the infiltration of macrophages, fibroblasts, and such, or due to other causes. The present inventors focused on the functions of chondroitin sulfate proteoglycans in order to establish that the improvement of degenerative conditions in cerebral nerve cells is an effective therapeutic method for Parkinson's disease. The inventors then suppressed the accumulation of chondroitin sulfate proteoglycans in a mouse model for Parkinson's disease, and closely analyzed this condition to reveal that the accumulation of chondroitin sulfate proteoglycans was ameliorated in many cells, and inflammation and such were also improved as compared with that in the cerebral nerve cells of wild-type mice. Specifically, the inventors discovered that inhibiting the production or accumulation of chondroitin sulfate proteoglycans facilitated the amelioration of abnormal accumulations of chondroitin sulfate proteoglycans in cerebral nerve cells, a factor deeply involved in Parkinson's disease, and thus leading to the improvement of neural fibrotic degeneration.

The present invention relates to neural fibrotic degeneration-suppressing agents, which comprise substances that inhibit the production or accumulation of chondroitin sulfate proteoglycans as active ingredients.

In the present invention, “chondroitin sulfate proteoglycans” are a type of proteoglycan and collectively refer to compounds in which proteins (core proteins) are covalently linked to chondroitin sulfate/dermatan sulfate, which are representative sulfated mucopolysaccharides. In the present invention, preferable “chondroitin sulfate proteoglycans” are human chondroitin sulfate proteoglycans. The species from which the proteoglycans are derived are not particularly limited. Proteins of nonhuman organisms (homologues, orthologues, and such) that are equivalent to the chondroitin sulfate proteoglycans are also included in the chondroitin sulfate proteoglycans of the present invention. For example, the present invention can be conducted as long as the species has a protein corresponding to a human chondroitin sulfate proteoglycan and equivalent to a human chondroitin sulfate proteoglycan. Furthermore, the chondroitin sulfate proteoglycans in the present invention also include so-called part-time proteoglycans, which are temporarily linked with glycosaminoglycan (GAG) chains to become proteoglycans upon inflammation or the like.

Examples of the chondroitin sulfate proteoglycans described below are aggrecan, versican, neurocan, brevican, β-glycan, decorin, biglycan, fibromodulin, and PG-Lb. However, the chondroitin sulfate proteoglycans in the present invention are not limited to these examples, and may be any substances with a chondroitin sulfate proteoglycan activity. Herein, chondroitin sulfate proteoglycan activities include, for example, cell adhesion ability and cell growth promotion. Those skilled in the art can assay chondroitin sulfate proteoglycan activities by assaying cell division and growth of tumor cells (for example, Caco-2 and HT-29 cells) in the presence of a protein containing a partial amino acid sequence of a chondroitin sulfate proteoglycan, or a protein with high homology to such a partial amino acid (typically 70% homology or higher, preferably 80% or higher, more preferably 90% or higher, and most preferably 95% or higher). Proteins with the effect of promoting cell division and growth can be evaluated as proteins with a chondroitin sulfate proteoglycan activity (Int. J. Exp. Pathol. 2005 August; 86 (4):219-29; Histochem Cell Biol. 2005 August; 124 (2):139-49). High homology means 50% or higher homology, preferably 70% or higher homology, more preferably 80% or higher homology, and still more preferably 90% or higher homology (for example, 95% or higher homology, or 96%, 97%, 98%, 99% or higher homology). Such homology can be determined using the MBLAST algorithm (Altschul et al. (1990) Proc. Natl. Acad. Sci. USA 87: 2264-8; Karlin and Altschul (1993) Proc. Natl. Acad. Sci. USA 90: 5873-7).

Herein, “neural fibrotic degeneration” refers to abnormal nerve tissue conditions. The conditions include, for example, fibrosis; inflammation; infiltration of fibroblasts, inflammatory cells or such; and loss or cell death of specific cell types in nerve tissues, but are not limited thereto.

Herein, “inhibition of production or accumulation” of chondroitin sulfate proteoglycans includes, for example, “promotion of degradation” “inhibition of synthesis”, “desulfation”, and “inhibition of sulfation” of chondroitin sulfate proteoglycans; however, “inhibition of production or accumulation” is not limited thereto, and it refers to a reduction or loss of the amount, function or activity of a chondroitin sulfate proteoglycan, as compared to a comparison subject. Herein, “substances that inhibit the production or accumulation” of chondroitin sulfate proteoglycans are not particularly limited. Such substances are preferably “substances with the effect of promoting the degradation of chondroitin sulfate proteoglycans”, “substances with the effect of inhibiting the synthesis of chondroitin sulfate proteoglycans”, “substances with the effect of desulfating chondroitin sulfate proteoglycans”, or “substances with the effect of inhibiting the sulfation of chondroitin sulfate proteoglycans”.

“Promotion of degradation” of chondroitin sulfate proteoglycans includes, for example, inhibition of the expression of core proteins of chondroitin sulfate proteoglycans, and a reduction in the abundance of the core proteins. Herein, “core proteins of chondroitin sulfate proteoglycans” include, for example, aggrecan, versican, neurocan, and brevican for matrix-type chondroitin sulfate proteoglycans; and β-glycan, decorin, biglycan, fibromodulin, and PG-Lb for membrane chondroitin sulfate proteoglycans. Those described above are all examples, but the core proteins are not limited to these and a wide variety of proteins may serve as chondroitin sulfate proteoglycan cores.

“Expression” includes “transcription” from genes, “translation” into polypeptides, and “suppression of degradation” of proteins. The “expression of core proteins of chondroitin sulfate proteoglycans” refers to transcription and translation of the genes encoding core proteins of chondroitin sulfate proteoglycans, or production of core proteins of chondroitin sulfate proteoglycans through transcription and translation. Furthermore, the “function of core proteins of chondroitin sulfate proteoglycans” includes, for example, their function in binding to chondroitin sulfate, and in binding with other cellular components. Those skilled in the art can appropriately evaluate (measure) the various above-mentioned functions using general methods. Specifically, the evaluation can be performed using the methods described in the Examples herein below, or using the same methods with appropriate modifications.

The “promotion of degradation” of chondroitin sulfate proteoglycans may also be an increase in the expression of enzymes that cleave or degrade chondroitin sulfate proteoglycans, or of enzymes involved in the cleavage or degradation of proteoglycans. Such enzymes include, for example, metalloproteinases (for example, ADAMTS-1, ADAMTS-4, and ADAMTS-5), chondroitinase, and calpain I, but are not limited thereto. The “promotion of degradation” may be a reduction in the abundance of chondroitin sulfate proteoglycans caused by administering all or some of the enzymes.

Alternatively, “promotion of degradation” may be achieved by administering a substance that promotes the suppression of expression of chondroitin sulfate proteoglycans. Such substances include, for example, n-butylate, diethylcarbamazepine, tunicamycin, non-steroidal estrogen, and cyclofenil deiphenol, but are not limited thereto.

Preferred embodiments of the “substance with the activity of promoting degradation” include, for example, compounds (nucleic acids) selected from the group consisting of:

(a) antisense nucleic acids against transcripts of the genes encoding the core proteins of chondroitin sulfate proteoglycans, or portions thereof,
(b) nucleic acids with the ribozyme activity of specifically cleaving transcripts of genes encoding core proteins of chondroitin sulfate proteoglycans; and
(c) nucleic acids with the activity of using RNAi effect to inhibit the expression of genes encoding core proteins of chondroitin sulfate proteoglycans.

Furthermore, the “substances with the activity of promoting degradation” include, for example, compounds selected from the group consisting of:

(a) antibodies that bind to core proteins of chondroitin sulfate proteoglycans;
(b) chondroitin sulfate proteoglycan variants that are dominant-negative for core proteins of chondroitin sulfate proteoglycans; and
(c) low-molecular-weight compounds that bind to core proteins of chondroitin sulfate proteoglycans.

“Inhibition of synthesis” of chondroitin sulfate proteoglycans includes, for example, inhibition of biosynthesis of glycosaminoglycans and inhibition of enzymes involved in the synthesis of chondroitin sulfate proteoglycans, but is not limited thereto. The inhibition refers to inhibition of any of the processes of chondroitin sulfate proteoglycan synthesis.

As substances that inhibit the synthesis of chondroitin sulfate proteoglycans, substances inhibiting the biosynthesis of glycosaminoglycans include, for example, β-D-xyloside, 2-deoxy-D-glucose (2-DG), ethane-1-hydroxy-1,1-diphosphonate (ETDP), and 5-hexyl-2-deoxyuridine (HudR). Such substances inhibit the biosynthesis of glycosaminoglycans, and thereby inhibit the synthesis of chondroitin sulfate proteoglycans.

Meanwhile, enzymes involved in chondroitin synthesis include, for example, GalNAc4ST-1, GalNAc4ST-2, GALNAC4S-6ST, UA20ST, GalT-I, GalT-II, GlcAT-I, and XylosylT. The synthesis of chondroitin sulfate proteoglycans is inhibited by inhibiting such enzymes, suppressing the expression thereof, or the like.

Preferred embodiments of “substances with the activity of inhibiting synthesis” include, for example, compounds (nucleic acids) selected from the group consisting of:

(a) antisense nucleic acids against transcripts of genes encoding chondroitin sulfate proteoglycan synthetases, or portions thereof;
(b) nucleic acids with the ribozyme activity of specifically cleaving transcripts of the genes encoding chondroitin sulfate proteoglycan synthetases; and
(c) nucleic acids with the activity of using RNAi effect to inhibit the expression of genes encoding chondroitin sulfate proteoglycan synthetases.

The “substances with the activity of inhibiting synthesis” also include, for example, compounds selected from the group consisting of:

(a) antibodies that bind to chondroitin sulfate proteoglycan synthetases;
(b) chondroitin sulfate proteoglycan synthetase variants that are dominant-negative for chondroitin sulfate proteoglycan synthetases; and
(c) low-molecular-weight compounds that bind to chondroitin sulfate proteoglycan synthetases.

The “desulfation” of chondroitin sulfate proteoglycans refers to the removal of a sulfate group from chondroitin sulfate proteoglycans, and includes, for example, desulfation by an endogenous or exogenously-administered desulfation enzyme, and suppression of sulfation by a sulfation-suppressing compound, but is not limited thereto. “Desulfation” refers to the process of sulfate group removal.

Such desulfation enzymes include, for example, chondroitin-4-sulfatase and chondroitin-6-sulfatase. Sulfation-suppressing compounds include, for example, chlorate and EGF receptor antagonists.

Preferred embodiments of such “substances with desulfating activity” include, for example, compounds (nucleic acids) selected from the group consisting of:

(a) antisense nucleic acids against transcripts of genes encoding proteins that suppress chondroitin sulfate proteoglycan-desulfating enzymes, or portions thereof;
(b) nucleic acids with the ribozyme activity of specifically cleaving transcripts of the genes encoding proteins that suppress chondroitin sulfate proteoglycan-desulfating enzymes; and
(c) nucleic acids with the activity of using RNAi effect to inhibit the expression of genes encoding proteins that suppress chondroitin sulfate proteoglycan-desulfating enzymes.

The “substances with desulfating activity” also include, for example, compounds selected from the group consisting of:

(a) antibodies that bind to compounds that suppress chondroitin sulfate proteoglycan-desulfating enzymes;
(b) variants of proteins that suppress chondroitin sulfate proteoglycan-desulfating enzymes, which are dominant-negative for proteins that suppress chondroitin sulfate proteoglycan-desulfating enzymes; and
(c) low-molecular-weight compounds that bind to compounds that suppress chondroitin sulfate proteoglycan-desulfating enzymes.

Herein, “desulfation-suppressing compounds” include not only proteins but also non-pertinacious compounds such as coenzymes.

The “activity of inhibiting sulfation” of chondroitin sulfate proteoglycans includes, for example, inhibition of sulfotransferases, but is not limited thereto. The activity refers to the inhibition of sulfation in the process of chondroitin sulfate proteoglycan synthesis.

Such sulfotransferases include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2.

Preferred embodiments of “substances with the activity of inhibiting sulfation” include, for example, compounds (nucleic acids) selected from the group consisting of:

(a) antisense nucleic acids against transcripts of genes encoding sulfotransferases for chondroitin sulfate proteoglycans, or portions thereof;
(b) nucleic acids with the ribozyme activity of specifically cleaving transcripts of the genes encoding sulfotransferases for chondroitin sulfate proteoglycans; and
(c) nucleic acids with the activity of using RNAi effect to inhibit the expression of genes encoding sulfotransferases for chondroitin sulfate proteoglycans.

The “substances with the activity of inhibiting sulfation” also include, for example, compounds selected from the group consisting of:

(a) antibodies that bind to sulfotransferases for chondroitin sulfate proteoglycans;
(b) sulfotransferase variants for chondroitin sulfate proteoglycans; and
(c) low-molecular-weight compounds that bind to sulfotransferases for chondroitin sulfate proteoglycans.

The above enzymes shown as examples include not only single enzymes that correspond to single genes, but also groups of enzymes that share certain characteristics. For example, chondroitinase is a collective name for enzymes such as ABC, AC, and B, whose substrate specificities or such are different, but which share the characteristics of mucopolysaccharide-degrading enzymes. For example, chondroitinase AC I eliminatively cleaves the N-acetylhexosaminide linkages of chondroitin sulfates (A, C, and E), chondroitin, chondroitin sulfate-dermatan sulfate hybrids, and hyaluronic acid, and yields oligosaccharides with Δ4-glucuronate residues at the non-reducing ends. This enzyme does not act on dermatan sulfate (chondroitin sulfate B, which has L-iduronic acid for a hexuronic acid), keratan sulfate, heparan sulfate, and heparin. Meanwhile, chondroitinase AC II eliminatively cleaves the N-acetylhexosaminide linkages of chondroitin, chondroitin sulfate A, and chondroitin sulfate C, and yields Δ4-unsaturated disaccharides (ΔDi-0S, ΔDi-4S, and ΔDi-6S). This enzyme also acts well on hyaluronic acid. The enzyme does not act on dermatan sulfate (chondroitin sulfate B), which is therefore a competitive inhibitor of the enzyme. Chondroitinase B (dermatanase) eliminatively cleaves N-acetylhexosaminide linkages to L-iduronic acids in dermatan sulfate, and yields oligosaccharides (di- and tetra-saccharides) with Δ4-hexuronate residues at the non-reducing ends. This enzyme acts on neither chondroitin sulfate A nor chondroitin sulfate C, which are free of L-iduronic acid. Dermatan, which is a derivative of dermatan sulfate in which the sulfate group is removed, does not serve as a substrate for this enzyme. This enzyme preferentially cleaves portions of dermatan sulfate in which the second of the L-iduronic acid units are sulfated. Chondroitinase ABC eliminatively cleaves N-acetylhexosaminide linkages of chondroitin sulfate A, chondroitin sulfate C, dermatan sulfate, chondroitin, and hyaluronic acid, and yields mainly disaccharides with A4-hexuronate groups at the non-reducing ends. This enzyme does not act on keratan sulfate, heparin, and heparan sulfate. Chondroitinases collectively refer to enzymes sharing the characteristics of mucopolysaccharide-degrading enzymes while also having different characteristics as described above, and they are not limited to chondroitinase ACI, chondroitinase AC II, chondrotinase B. and chondroitinase ABC as exemplified above.

Further, on a genomic DNA level, such groups of enzymes sharing features do not necessarily correspond to single genes. For example, both chondroitin-4-sulfatase and chondroitin-6-sulfatase can be retrieved from the public gene database Genbank as sequences referred to by multiple accession numbers (for example, Genbank accession Nos: NT_{—}039500 (a portion thereof is shown under accession No: CAAA01098429 (SEQ ID NO: 83)), NT_{—}078575, NT_{—}039353, NW_{—}001030904, NW_{—}001030811, NW_{—}001030796, and NW_{—}000349).

The above example proteins that correspond to single genes are shown below: Specifically, below are the accession numbers in the public gene database Genbank, nucleotide sequences, and amino acid sequences for human genes encoding: aggrecan, versican, neurocan, brevican, β-glycan, decorin, biglycan, fibromodulin, and PG-Lb, which are shown above as examples of chondroitin sulfate proteoglycans; ADAMTS-1, ADAMTS-4, ADAMTS-5, and calpain I, which are shown above as examples of enzymes that cleave or degrade chondroitin sulfate proteoglycans or related enzymes; GalNAc4ST-1, GalNAc4ST-2, GALNAC4S-6ST, UA20ST, GalT-I, GalT-II, GlcAT-I, and XylosylT, which are shown above as examples of enzymes involved in chondroitin synthesis; C4ST-1, C4ST-2, C4ST-3, D4ST, C6ST-1, and C6ST-2, which are shown above as examples of sulfotransferases.

Aggrecan (Accession No: NM_{—}007424; nucleotide sequence: SEQ ID NO: 1; amino acid sequence: SEQ ID NO: 2)
Versican (Accession No: BC096495; nucleotide sequence: SEQ ID NO: 3; amino acid sequence: SEQ ID NO: 4)
Neurocan (Accession No: NM_{—}010875; nucleotide sequence: SEQ ID NO: 5; amino acid sequence: SEQ ID NO: 6)
Brevican (Accession No: NM_{—}007529; nucleotide sequence: SEQ ID NO: 7; amino acid sequence: SEQ ID NO: 8)
β-glycan (Accession No: AF039601; nucleotide sequence: SEQ ID NO: 9; amino acid sequence: SEQ ID NO: 10)
Decorin (Accession No: NM_{—}007833; nucleotide sequence: SEQ ID NO: 11; amino acid sequence: SEQ ID NO: 12)
Biglycan (Accession No: BC057185; nucleotide sequence: SEQ ID NO: 13; amino acid sequence: SEQ ID NO: 14)
Fibromodulin (Accession No: NM_{—}021355; nucleotide sequence: SEQ ID NO: 15; amino acid sequence: SEQ ID NO: 16)
PG-Lb (Accession No: NM_{—}007884; nucleotide sequence: SEQ ID NO: 17; amino acid sequence: SEQ ID NO: 18)
ADAMTS-1 (Accession No: NM_{—}009621; nucleotide sequence: SEQ ID NO: 19; amino acid sequence: SEQ ID NO: 20)
ADAMTS-4 (Accession No: NM_{—}172845; nucleotide sequence: SEQ ID NO: 21; amino acid sequence: SEQ ID NO: 22)
ADAMTS-5 (Accession No: AF140673; nucleotide sequence: SEQ ID NO: 23; amino acid sequence: SEQ ID NO: 24)
Calpain I (Accession No: NM_{—}007600; nucleotide sequence: SEQ ID NO: 25; amino acid sequence: SEQ ID NO: 26)
GalNAc4ST-1 (Accession No: NM_{—}175140; nucleotide sequence: SEQ ID NO: 27; amino acid sequence: SEQ ID NO: 28)
GalNAc4ST-2 (Accession No: NM_{—}199055; nucleotide sequence: SEQ ID NO: 29; amino acid sequence: SEQ ID NO: 30)
GALNAC4S-6ST (Accession No: NM_{—}029935; nucleotide sequence: SEQ ID NO: 31; amino acid sequence: SEQ ID NO: 32)
UA20ST (Accession No: NM_{—}177387; nucleotide sequence: SEQ ID NO: 33; amino acid sequence: SEQ ID NO: 34)
GalT-I (Accession No: NM_{—}016769; nucleotide sequence: SEQ ID NO: 35; amino acid sequence: SEQ ID NO: 36)
GalT-II (Accession No: BC064767; nucleotide sequence: SEQ ID NO: 37; amino acid sequence: SEQ ID NO: 38)
GlcAT-I (Accession No: BC058082; nucleotide sequence: SEQ ID NO: 39; amino acid sequence: SEQ ID NO: 40), or Accession No: NM_{—}024256; nucleotide sequence: SEQ ID NO: 41; amino acid sequence: SEQ ID NO: 42)
XylosylT (Accession No: NM_{—}145828; nucleotide sequence: SEQ ID NO: 43; amino acid sequence: SEQ ID NO: 44)
C4ST-1 (Accession No: NM_{—}021439; nucleotide sequence: SEQ ID NO: 45; amino acid sequence: SEQ ID NO: 46)
C4ST-2 (Accession No: NM_{—}021528; nucleotide sequence: SEQ ID NO: 47; amino acid sequence: SEQ ID NO: 48)
C4ST-3 (Accession No: XM_{—}355798; nucleotide sequence: SEQ ID NO: 49; amino acid sequence: SEQ ID NO: 50)
D4ST (Accession No: NM_{—}028117; nucleotide sequence: SEQ ID NO: 51; amino acid sequence: SEQ ID NO: 52)
C6ST-1 (Accession No: NM_{—}016803; nucleotide sequence: SEQ ID NO: 53; amino acid sequence: SEQ ID NO: 54)
C6ST-2 (Accession No: AB046929; nucleotide sequence: SEQ ID NO: 55; amino acid sequence: SEQ ID NO: 56)

In addition to the proteins listed above, the proteins of the present invention include those exhibiting high homology (typically 70% or higher, preferably 80% or higher, more preferably 90% or higher, and most preferably 95% or higher) to sequences shown in the Sequence Listing and with a function of the proteins listed above (for example, the function of binding to intracellular components). The proteins listed above are, for example, proteins comprising an amino acid sequence with an addition, deletion, substitution, or insertion of one or more amino acids in any of the amino acid sequences of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, and 56, in which the number of altered amino acids is typically 30 amino acids or less, preferably ten amino acids or less, more preferably five amino acids or less, and most preferably three amino acids or less.

The above-described genes of the present invention include, for example, endogenous genes of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 (homologues to the human genes described above, or the like).

Each of the endogenous DNAs of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 are generally highly homologous to a DNA of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55. High homology means 50% or higher homology, preferably 70% or higher homology, more preferably 80% or higher homology, and still more preferably 90% or higher homology (for example, 95% or higher, or 96%, 97%, 98%, or 99% or higher). Homology can be determined using the MBLAST algorithm (Altschul et al. (1990) Proc. Natl. Acad. Sci. USA 87: 2264-8; Karlin and Altschul (1993) Proc. Natl. Acad. Sci. USA 90: 5873-7). When the DNAs have been isolated from the body, each of them may hybridize under stringent conditions to a DNA of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, or 55. Herein, stringent conditions include, for example, “2×SSC, 0.1% SDS, 50° C.”, “2×SSC, 0.1% SDS, 42° C.”, and “1×SSC, 0.1% SDS, 37° C.”; more stringent conditions include “2×SSC, 0.1% SDS, 65° C.”, “0.5×SSC, 0.1% SDS, 42° C.”, and “0.2×SSC, 0.1% SDS, 65° C.”.

Those skilled in the art can appropriately obtain proteins functionally equivalent to the above-described proteins from the above-described highly homologous proteins by using methods for assaying the activity of promoting the degradation of CSPGs, inhibiting the synthesis of CSPGs, desulfating CSPGs, or inhibiting the sulfation of CSPGs. Specific methods for assaying the activities are described below in a section on the screening methods of the present invention. Further, based on the nucleotide sequences of the above-described genes, those skilled in the art can appropriately obtain endogenous genes of other organisms that correspond to the above-described genes. In the present invention, the above-described proteins and genes in non-human organisms, which correspond to the above-described proteins and genes, or the above-described proteins and genes that are functionally equivalent to the above-described proteins and genes, may simply be referred to using the above-described names.

The proteins of the present invention can be prepared not only as natural proteins but also as recombinant proteins using genetic recombination techniques. The natural proteins can be prepared by, for example, methods of subjecting cell extracts (tissue extracts) that may express the above-described proteins to affinity chromatography using antibodies against the above-described proteins. On the other hand, the recombinant proteins can be prepared, for example, by culturing cells transformed with DNAs encoding the proteins described above. The above-described proteins of the present invention can be suitably used, for example, in the screening methods described herein below.

In the present invention, “nucleic acids” refer to both RNAs and DNAs. Chemically synthesized nucleic acid analogs, such as so-called “PNAs” (peptide nucleic acids), are also included in the nucleic acids of the present invention. PNAs are nucleic acids in which the fundamental backbone structure of nucleic acids, the pentose-phosphate backbone, is replaced by a polyamide backbone with glycine units. PNAs have a three-dimensional structure quite similar to that of nucleic acids.

Methods for inhibiting the expression of specific endogenous genes using antisense technology are well known to those skilled in the art. There are a number of causes for the action of antisense nucleic acids in inhibiting target gene expression, including:

inhibition of transcription initiation by triplex formation;
transcription inhibition by hybrid formation at a site with a local open loop structure generated by an RNA polymerase;
transcription inhibition by hybrid formation with the RNA being synthesized;
splicing inhibition by hybrid formation at an intron-exon junction;
splicing inhibition by hybrid formation at the site of spliceosome formation;
inhibition of transport from the nucleus to the cytoplasm by hybrid formation with mRNA;
splicing inhibition by hybrid formation at the capping site or poly(A) addition site;
inhibition of translation initiation by hybrid formation at the translation initiation factor binding site;
inhibition of translation by hybrid formation at the ribosome binding site adjacent to the start codon;
inhibition of peptide chain elongation by hybrid formation in the translational region of mRNA or at the polysome binding site of mRNA; and
inhibition of gene expression by hybrid formation at the protein-nucleic acid interaction sites.
Thus, antisense nucleic acids inhibit the expression of target genes by inhibiting various processes, such as transcription, splicing, and translation ((Hirashima and Inoue, Shin Seikagaku Jikken Koza 2 (New Courses in Experimental Biochemistry 2), Kakusan (Nucleic Acids) TV: “Idenshi no Fukusei to Hatsugen (Gene replication and expression)”, Ed. The Japanese Biochemical Society, Tokyo Kagakudojin, pp. 319-347 (1993)).

The antisense nucleic acids used in the present invention may inhibit the expression and/or function of genes encoding any of the CSPG core proteins, synthetases, proteins suppressing desulfation enzymes, and sulfotransferases described above, based on any of the actions described above. In one embodiment, antisense sequences designed to be complementary to an untranslated region adjacent to the 5′ end of an mRNA for a gene encoding an above-described CSPG core protein, synthetase, desulfation enzyme-suppressing protein, or sulfotransferase may be effective for inhibiting translation of the gene. Sequences complementary to a coding region or 3′-untranslated region can also be used. Thus, the antisense nucleic acids to be used in the present invention include not only nucleic acids comprising sequences antisense to the coding regions, but also nucleic acids comprising sequences antisense to untranslated regions of genes encoding the above-described CSPG core proteins, synthetases, desulfation enzyme-suppressing proteins, or sulfotransferases. Such antisense nucleic acids to be used are linked downstream of adequate promoters and are preferably linked with transcription termination signals on the 3′ side. Nucleic acids thus prepared can be introduced into desired animals (cells) using known methods. The sequences of the antisense nucleic acids are preferably complementary to a gene or portion thereof encoding a CSPG core protein, synthetase, desulfation enzyme-suppressing protein, or sulfotransferase that is endogenous to the animals (cells) to be transformed with them. However, the sequences need not be perfectly complementary, as long as the antisense nucleic acids can effectively suppress expression of a gene. The transcribed RNAs preferably have 90% or higher, and most preferably 95% or higher complementarity to target gene transcripts. To effectively inhibit target gene expression using antisense nucleic acids, the antisense nucleic acids are preferably at least 15 nucleotides long, and less than 25 nucleotides long. However, the lengths of the antisense nucleic acids of the present invention are not limited to the lengths mentioned above, and they may be 100 nucleotides or more, or 500 nucleotides or more.

The antisense nucleic acids of the preset invention are not particularly limited, and can be prepared, for example, based on the nucleotide sequence of a versican gene (GenBank Accession No: BC096495; SEQ ID NO: 3), C4ST-1 (GenBank Accession No: NM_{—}021439; SEQ ID NO: 45), C4ST-2 (GenBank Accession NO: NM_{—}021528; SEQ ID NO: 47), C4ST-3 (GenBank Accession NO: XM_{—}355798; SEQ ID NO: 49), or such.

Expression of the above-mentioned genes encoding CSPG core proteins, synthetases, desulfation enzyme-suppressing proteins, or sulfotransferases can also be inhibited using ribozymes or ribozyme-encoding DNAs. Ribozymes refer to RNA molecules with catalytic activity. There are various ribozymes with different activities. Among others, studies that focused on ribozymes functioning as RNA-cleaving enzymes have enabled the design of ribozymes that cleave RNAs in a site-specific manner. Some ribozymes have 400 or more nucleotides, such as group I intron type ribozymes and M1 RNA, which is comprised by RNase P. but others, called hammerhead and hairpin ribozymes, have a catalytic domain of about 40 nucleotides (Koizumi, M. and Otsuka E., Tanpakushitsu Kakusan Koso (Protein, Nucleic Acid, and Enzyme) 1990, 35, 2191).

For example, the autocatalytic domain of a hammerhead ribozyme cleaves the sequence G13U14C15 at the 3′ side of C15. Base pairing between U14 and A9 has been shown to be essential for this activity, and the sequence can be cleaved when C15 is substituted with A15 or U15 (Koizumi, M. et al., FEBS Lett, 1988, 228, 228). Restriction enzyme-like RNA-cleaving ribozymes that recognize the sequence UC, ULU, or UA in target RNAs can be created by designing their substrate-binding sites to be complementary to an RNA sequence adjacent to a target site (Koizumi, M. et al., FEBS Lett, 1988, 239, 285; Koizumi, M, and Otsuka E., Tanpakushitsu Kakusan Koso (Protein, Nucleic Acid, and Enzyme) 1990, 35, 2191; and Koizumi, M. et al., Nucl Acids Res, 1989, 17, 7059).

In addition, hairpin ribozymes are also useful for the purposes of the present invention. Such ribozymes are found in, for example, the minus strand of satellite RNAs of tobacco ringspot viruses (Buzayan, J M., Nature, 1986, 323, 349). It has been shown that target-specific RNA-cleaving ribozymes can also be created from hairpin ribozymes (Kikuchi, Y. and Sasaki, N., Nucl Acids Res, 1991, 19, 6751; and Kikuchi Y. Kagaku to Seibutsu (Chemistry and Biology) 1992, 30, 112). Thus, the expression of the above-described genes encoding CSPG core proteins, synthetases, desulfation enzyme-suppressing proteins, or sulfotransferases can be inhibited by using ribozymes to specifically cleave the gene transcripts.

The expression of endogenous genes can also be suppressed by RNA interference (hereinafter abbreviated as “RNAi”), using double-stranded RNAs comprising a sequence the same as or similar to a target gene sequence.

A great many disease-related genes have been rapidly identified since the entire human nucleotide sequence was revealed upon the recent completion of the genome project, and currently specific gene-targeted therapies and drugs are being actively developed. Of these, the application to gene therapy of small interfering RNAs (siRNAs), which produce the effect of specific post-transcriptional suppression, has been drawing attention. RNAi is a technology currently drawing attention in which double-stranded RNAs (dsRNAs) incorporated directly into cells suppress the expression of genes with sequences homologous to the dsRNAs. In mammalian cells, RNAi can be induced using short dsRNAs (siRNAs) and has many advantages: compared to knockout mice, RNAi has a stable effect, simple experiments, low costs, and so on.

Nucleic acids with inhibitory activity based on RNAi effect are generally referred to as siRNAs or shRNAs. RNAi is a phenomenon in which, when cells or such are introduced with short double-stranded RNAs (hereinafter abbreviated as “dsRNAs”) comprising sense RNAs that comprise sequences homologous to the mRNAs of a target gene, and antisense RNAs that comprise sequences homologous a sequence complementary thereto, the dsRNAs bind specifically and selectively to the target gene mRNAs, induce their disruption, and cleave the target gene, thereby effectively inhibiting (suppressing) target gene expression. For example, when dsRNAs are introduced into cells, the expression of genes with sequences homologous to the RNAs is suppressed (the genes are knocked down). As described above, RNAi can suppress the expression of target genes, and is thus drawing attention as a method applicable to gene therapy, or as a simple gene knockout method replacing conventional methods of gene disruption, which are based on complicated and inefficient homologous recombination. The RNAs to be used in RNAi are not necessarily perfectly identical to the genes or portions thereof that encode an above-described CSPG core protein, synthetase, desulfation enzyme-suppressing protein, or sulfotransferase; however, the RNAs are preferably perfectly homologous to the genes or portions thereof.

The targets of the siRNAs to be designed are not particularly limited, as long as they are genes encoding an above-described CSPG core protein, synthetase, desulfation enzyme-suppressing protein, or sulfotransferase. Any region of the gene can be a candidate for a target. For example, siRNAs may be prepared based on a nucleotide sequence of the versican gene (SEQ ID NO: 3), C4ST-1 gene (SEQ ID NO: 45), C4ST-2 gene (SEQ ID NO: 47), C4ST-3 gene (SEQ ID NO: 49), and such. More specifically, partial regions of such sequences may be used as candidates for the targets. For example, siRNAs may be prepared based on portions of the nucleotide sequences of a versican gene (SEQ ID NO: 57), C4ST-1 gene (SEQ ID NO: 58), C4ST-2 gene (SEQ ID NO: 59), C4ST-3 gene (SEQ ID NO: 60), C6ST-1 gene (SEQ ID NO: 61), C6ST-2 gene (SEQ ID NO: 62), GalNAc4ST-1 gene (SEQ ID NO: 63), GalNAc4ST-2 gene (SEQ ID NO: 64), GALNAC4S-6ST gene (SEQ ID NO: 65), or such. More specifically, examples of the siRNAs also include those targeted to the DNA sequences (SEQ ID NOs: 71 to 82) specifically shown herein.

The siRNAs can be introduced into cells by adopting methods of introducing cells with plasmid DNAs linked with siRNAs synthesized in vitro or methods that comprise annealing two RNA strands.

The two RNA molecules described above may be closed at one end or, for example, may be siRNAs with hairpin structures (shRNAs). shRNAs refer to short hairpin RNAs, which are RNA molecules with a stem-loop structure, since a portion of the single strand constitutes a strand complementary to another portion. Thus, molecules capable of forming an intramolecular RNA duplex structure are also included in the siRNAs of the present invention.

In a preferred embodiment of the present invention, the siRNAs of the present invention also include, for example, double-stranded RNAs with additions or deletions of one or a few RNAs in an siRNA which targets a specific DNA sequence (SEQ ID NOs: 71 to 82) shown herein and which can suppress the expression of versican, C4ST-1, C4ST-2, C4ST-3, or such via RNAi effect, as long as the double-stranded RNAs have the function of suppressing the expression of a gene encoding an above-described CSPG core protein, synthetase, desulfation enzyme-suppressing protein, or sulfotransferase.

The RNAs used in RNAi (siRNAs) do not need to be perfectly identical (homologous) to the genes encoding the above proteins or portions thereof, however, the RNAs are preferably perfectly identical (homologous).

Some details of the RNAi mechanism still remain unclear, but it is understood that an enzyme called “DICER” (a member of the RNase III nuclease family) is contacted with a double-stranded RNA and degrades it in to small fragments, called “small interfering RNAs” or “siRNAs”. The double-stranded RNAs of the present invention that have RNAi effect include such double-stranded RNAs prior to being degraded by DICER. Specifically, since even long RNAs that have no RNAi effect when intact can be degraded into siRNAs which have RNAi effect in cells, the length of the double-stranded RNAs of the present invention is not particularly limited.

For example, long double-stranded RNAs covering the full-length or near full-length mRNA of a gene encoding an above-described CSPG core protein, synthetase, desulfation enzyme-suppressing protein, or sulfotransferase can be pre-digested, for example, by DICER, and then the degradation products can be used as agents of the present invention. These degradation products are expected to contain double-stranded RNA (siRNA) molecules with RNAi effect. With this method, it is not necessary to specifically select the mRNA regions expected to have RNAi effect. In other words, it is not necessary to accurately determine regions with RNAi effect in the mRNAs of the genes described above.

The above-described “double-stranded RNAs capable of suppression via RNAi effect” can be suitably prepared by those skilled in the art based on nucleotide sequences of the above-described CSPG genes encoding core proteins, synthetases, desulfation enzyme-suppressing proteins, or sulfotransferases, which are targeted by the double-stranded RNAs. For example, the double-stranded RNAs of the present invention can be prepared based on the nucleotide sequence of SEQ ID NO: 71. In other words, it is within the range of ordinary trials for those skilled in the art to select an arbitrary consecutive RNA region in an mRNA that is a transcript of the nucleotide sequence of SEQ ID NO: 71, and prepare double-stranded RNA corresponding to the region. Those skilled in the art can also use known methods to properly select siRNA sequences with stronger RNAi effect from the mRNA sequence, which is the transcript of the nucleotide sequence of SEQ ID NO: 71. When one of the strands is already identified, those skilled in the art can readily determine the nucleotide sequence of the other strand (complementary strand). Those skilled in the art can appropriately prepare siRNAs using a commercially available nucleic acid synthesizer. Alternatively, general custom synthesis services may be used to synthesize desired RNAs.

The siRNAs of the present invention are not necessarily single pairs of double-stranded RNAs directed to target sequences, but may be mixtures of multiple double-stranded RNAs directed to regions that cover the target sequence. Herein, those skilled in the art can appropriately prepare the siRNAs as nucleic acid mixtures matched to a target sequence by using a commercially available nucleic acid synthesizer or DICER enzyme. Meanwhile, general custom synthesis services may be used to synthesize desired RNAs. The siRNAs of the present invention include so-called “siRNA cocktails”.

All nucleotides in the siRNAs of the present invention do not necessarily need to be ribonucleotides (RNAs). Specifically, one or more of the ribonucleotides constituting the siRNAs of the present invention may be replaced with corresponding deoxyribonucleotides. The term “corresponding” means that although the sugar moieties are structurally differently, the nucleotide residues (adenine, guanine, cytosine, or thymine (uracil)) are the same. For example, deoxyribonucleotides corresponding to ribonucleotides with adenine refer to deoxyribonucleotides with adenine. The term “or more” described above is not particularly limited, but preferably refers to a small number of about two to five ribonucleotides.

Furthermore, DNAs (vectors) capable of expressing the RNAs of the present invention are also included in the preferred embodiments of compounds capable of suppressing the expression of the genes encoding the above-described proteins of the present invention. The DNAs (vectors) capable of expressing the double-stranded RNAs of the present invention are, for example, DNAs structured such that a DNA encoding one strand of a double-stranded RNA and a DNA encoding the other strand of the double-stranded RNA are linked with promoters so that each DNA can be expressed. The above DNAs of the present invention can be appropriately prepared by those skilled in the art using standard genetic engineering techniques. More specifically, the expression vectors of the present invention can be prepared by adequately inserting DNAs encoding the RNAs of the present invention into various known expression vectors.

Furthermore, the expression-inhibiting substances of the present invention also include compounds that inhibit the expression of the above-described CSPG core proteins, synthetases, desulfation enzyme-suppressing proteins, or sulfotransferases by binding to an expression regulatory region of a gene encoding the above-described CSPG core proteins, synthetases, desulfation enzyme-suppressing proteins, or sulfotransferases (for example, a promoter region; specific examples include the nucleotide sequence of SEQ ID NO: 66, which is a promoter region of PG-Lb). Such compounds can be obtained, for example, using a fragment of a promoter DNA of the gene encoding an above-described CSPG core protein, synthetase, desulfation enzyme-suppressing protein, or sulfotransferase to perform screening methods using as an indicator the activity of binding to the DNA fragment. Those skilled in the art can appropriately determine whether compounds of interest inhibit the expression of the above-described genes encoding CSPG core proteins, synthetases, desulfation enzyme-suppressing proteins, or sulfotransferases by using known methods, for example, reporter assays and such.

Furthermore, DNAs (vectors) capable of expressing the above-described RNAs of the present invention are also included in preferred embodiments of the compounds capable of inhibiting the expression of a gene encoding an above-described CSPG core protein, synthetase, desulfation enzyme-suppressing protein, or sulfotransferase of the present invention. For example, DNAs (vectors) capable of expressing the above-described double-stranded RNAs of the present invention are structured such that a DNA encoding one strand of a double-stranded RNA and a DNA encoding the other strand of the double-stranded RNA are linked to promoters so that both can be expressed. Those skilled in the art can appropriately prepare the above-described DNAs of the present invention using standard genetic engineering techniques. More specifically, the expression vectors of the present invention can be prepared by appropriately inserting DNAs encoding the RNAs of the present invention into various known expression vectors.

Preferred embodiments of the above-described vector of the present invention include vectors expressing RNAs (siRNAs) that can suppress the expression of versican, C4ST-1, C4ST-2, C4ST-3, or the like by RNAi effect.

Antibodies that bind to the above-described CSPG core proteins, synthetases, desulfation enzyme-suppressing compounds, or sulfotransferases can be prepared by methods known to those skilled in the art. Polyclonal antibodies can be obtained, for example, by the following procedure: small animals such as rabbits are immunized with an above-described natural protein or a recombinant protein expressed in microorganisms as a fusion protein with GST, or a partial peptide thereof. Sera are obtained from these animals and purified by, for example, ammonium sulfate precipitation, Protein A or G column, DEAE ion exchange chromatography, affinity column coupled with the core protein, synthetase, desulfation enzyme-suppressing compound, or sulfotransferase for CSPGs described above, synthetic peptide, or such, to prepare antibodies. Monoclonal antibodies can be obtained by the following procedure: small animals such as mice are immunized with an above-described CSPG core protein, synthetase, desulfation enzyme-suppressing compound, or sulfotransferase, or a partial peptide thereof. Spleens are removed from the mice and crushed to isolate cells. The cells are fused with mouse myeloma cells using a reagent such as polyethylene glycol. Clones producing antibodies that bind to an above-described CSPG core protein, synthetase, desulfation enzyme-suppressing compound, or sulfotransferase are selected from among the resulting fused cells (hybridomas). The obtained hybridomas are then transplanted in the peritoneal cavities of mice, and ascites is collected from the mice. The obtained monoclonal antibodies can be purified by, for example, ammonium sulfate precipitation, Protein A or G columns, DEAE ion exchange chromatography, affity columns coupled with an above-described CSPG core protein, synthetase, desulfation enzyme-suppressing compound, or sulfotransferase, synthetic peptides, or such.

The antibodies of the present invention are not particularly limited as long as they bind to an above-described core protein, synthetase, desulfation enzyme-suppressing compound, or sulfotransferase of the present invention. The antibodies of the present invention may be human antibodies, humanized antibodies created by gene recombination, fragments or modified products of such antibodies, in addition to the polyclonal and monoclonal antibodies described above.

The proteins of the present invention used as sensitizing antigens to prepare antibodies are not limited in terms of the animal species from which the proteins are derived. However, the proteins are preferably derived from mammals, for example, mice and humans.

Human-derived proteins are particularly preferred. The human-derived proteins can be appropriately obtained by those skilled in the art using the gene or amino acid sequences disclosed herein.

In the present invention, the proteins to be used as sensitizing antigens may be whole proteins or partial peptides thereof. Such partial peptides of the proteins include, for example, amino-terminal (N) fragments and carboxyl-terminal (C) fragments of the proteins. Herein, “antibodies” refer to antibodies that react with a full-length protein or fragment thereof.

In addition to immunizing nonhuman animals with antigens to obtain the above hybridomas, human lymphocytes, for example, EB virus-infected human lymphocytes, can be sensitized in vitro with the proteins or with cells expressing the proteins, or with lysates thereof, and the sensitized lymphocytes can be fused with human-derived myeloma cells with the ability to divide permanently, for example, U266, to obtain hybridomas that produce desired human antibodies with binding activity to the proteins.

It is expected that antibodies against the above-described CSPG core proteins, synthetases, desulfation enzyme-suppressing compounds, or sulfotransferases of the present invention exhibit the effect of inhibiting protein expression or function by binding to the proteins. When using the prepared antibodies for human administration (antibody therapy), the antibodies are preferably human or humanized antibodies in order to reduce immunogenicity.

Furthermore, in the present invention, low-molecular-weight substances (low-molecular-weight compounds) that bind to the above-described CSPG core proteins, synthetases, desulfation enzyme-suppressing compounds, or sulfotransferases are also included in the substances capable of inhibiting the function of the above-described CSPG core proteins, synthetases, desulfation enzyme-suppressing compounds, or sulfotransferases. Such low-molecular-weight substances may be natural or artificial compounds. In general, the compounds can be produced or obtained by methods known to those skilled in the art. The compounds of the present invention can also be obtained by the screening methods described below.

In addition, the substances of the present invention capable of inhibiting the expression or function of the above-described CSPG core proteins, synthetases, desulfation enzyme-suppressing proteins, or sulfotransferases include dominant-negative mutants (dominant-negative proteins) for the above-described CSPG core proteins, synthetases, desulfation enzyme-suppressing proteins, or sulfotransferases. The “dominant-negative protein mutants for the above CSPG core proteins, synthetases, desulfation enzyme-suppressing proteins, or sulfotransferases” refer to proteins with the function of reducing or abolishing the activity of endogenous wild-type proteins by expressing the genes encoding the CSPG core proteins, synthetases, desulfation enzyme-suppressing proteins, or sulfotransferases. Such dominant-negative proteins include, for example, versican core protein mutants that competitively inhibit the linking of the wild-type versican core protein with chondroitin sulfate.

Furthermore, in the present invention, the organs, tissues, or cells where the production or accumulation of chondroitin sulfate proteoglycans is inhibited are not specifically limited; however, they are preferably organs and tissues containing nerve cells, and are more preferably the brain, brain tissues, or nerve cells in brain tissues.

Compounds that inhibit the production or accumulation of chondroitin sulfate proteoglycans are expected to serve as therapeutic or preventive agents for neural fibrotic degenerative diseases. Herein, “therapeutic or preventive” does not necessarily refer to a perfect therapeutic or preventive effect on organs, tissues, or cells with neural fibrotic degeneration, and may refer to a partial effect.

In the present invention, the neural fibrotic degenerative diseases are not specifically limited, as long as they are associated with neural fibrotic degeneration; however, the neural fibrotic degenerative diseases are preferably cerebral neural fibrotic degenerative diseases, more preferably cerebrospinal neural fibrotic degenerative diseases, or peripheral neural fibrotic degenerative diseases, even more preferably Parkinson's disease, Alzheimer's disease, amyotrophic lateral sclerosis, polyglutamine disease, myelopathic muscular atrophy, Huntington's disease, multiple sclerosis, and the like.

The neural fibrotic degeneration-suppressing agents of the present invention have the activity of suppressing neural fibrotic degeneration through inhibiting the production or accumulation of chondroitin sulfate proteoglycans, which is a cause of neural fibrotic degeneration. Thus, preferred embodiments of the present invention provide, for example, therapeutic agents for cerebral neural fibrotic degenerative diseases, therapeutic agents for cerebrospinal neural fibrotic degenerative diseases, therapeutic agents for peripheral neural fibrotic degenerative diseases, therapeutic agents for Parkinson's disease, therapeutic agents for Alzheimer's disease, therapeutic agents for amyotrophic lateral sclerosis, therapeutic agents for polyglutamine disease, therapeutic agents for myelopathic muscular atrophy, therapeutic agents for Huntington's disease, and therapeutic agents for multiple sclerosis, all of which comprise as an active ingredient a neural fibrotic degeneration-suppressing agent of the present invention.

The “neural fibrotic degeneration-suppressing agents” of the present invention can also be referred to as “therapeutic agents for neural fibrotic degeneration”, “neural fibrotic degeneration inhibitors”, “neural fibrotic degeneration-improving agents”, or the like. Meanwhile, the “suppressing agents” of the present invention can also be referred to as “pharmaceutical agents”, “pharmaceutical compositions”, “therapeutic medicines”, or the like.

The “treatments” of the present invention also comprise preventive effects that can suppress the onset of neural fibrotic degeneration in advance. The treatments are not limited to those producing a perfect therapeutic effect on cells (tissues) developing neural fibrotic degeneration, and the effects may be partial.

The agents of the present invention can be combined with physiologically acceptable carriers, excipients, diluents and such, and orally or parenterally administered as pharmaceutical compositions. Oral agents may be in the form of granules, powders, tablets, capsules, solutions, emulsions, suspensions, or the like. The dosage forms of parenteral agents can be selected from injections, infusions, external preparations, inhalants (nebulizers), suppositories, and the like. Injections include preparations for intracranial, intranasal, subcutaneous, intramuscular, and intraperitoneal injections, and the like. The external preparations include nasal preparations, ointments, and such. Techniques for formulating the above-described dosage forms that contain the agents of the present invention as primary ingredients are known.

For example, tablets for oral administration can be produced by compressing and shaping the agents of the present invention in combination with excipients, disintegrants, binders, lubricants, and the like. Excipients commonly used include lactose, starch, mannitol, and the like. Commonly used disintegrants include calcium carbonate, carboxymethylcellulose calcium, and the like. Binders include gum arabic, carboxymethylcellulose, and polyvinylpyrrolidone. Known lubricants include talc, magnesium stearate, and such.

Known coatings can be applied to tablets comprising the agents of the present invention to prepare enteric coated formulations or for masking. Ethylcellulose, polyoxyethylene glycol, or such can be used as a coating agent.

Meanwhile, injections can be prepared by dissolving the agents of the present invention, which are chief ingredients, together with an appropriate dispersing agent, or dissolving or dispersing the agents in a dispersion medium. Both water-based and oil-based injections can be prepared, depending on the selection of dispersion medium. When preparing water-based injections, the dispersing agent is distilled water, physiological saline, Ringer's solution or such. For oil-based injections, any of the various vegetable oils, propylene glycols, or such is used as a dispersing agent. If required, a preservative such as paraben may be added at this time. Known isotonizing agents such as sodium chloride and glucose can also be added to the injections. In addition, soothing agents such as benzalkonium chloride and procaine hydrochloride can be added.

Alternatively, the agents of the present invention can be formed into solid, liquid, or semi-solid compositions to prepare external preparations. Such solid or liquid compositions can be prepared as the same compositions as described above and then used as external preparations. The semi-solid compositions can be prepared using an appropriate solvent, to which a thickener is added if required. Water, ethyl alcohol, polyethylene glycol, and the like can be used as the solvent. Commonly used thickeners are bentonite, polyvinyl alcohol, acrylic acid, methacrylic acid, polyvinylpyrrolidone, and the like. Preservatives such as benzalkonium chloride can be added to these compositions. Alternatively, suppositories can be prepared by combining the compositions with carriers, like oil bases such as cacao butter, or aqueous gel bases such as cellulose derivatives.

When the agents of the present invention are used as gene therapy agents, the agents may be directly administered by injection, or vectors carrying the nucleic acid may be administered. Such vectors include adenovirus vectors, adeno-associated virus vectors, herpes virus vectors, vaccinia virus vectors, retroviral vectors, and lentivirus vectors. These vectors allow efficient administration.

Alternatively, the agents of the present invention can be encapsulated into phospholipid vesicles such as liposomes, and then the vesicles can be administered. Vesicles carrying siRNAs or shRNAs are introduced into given cells by lipofection. The resulting cells are then systemically administered, for example, intravenously or intra-arterially. The cells can also be locally administered into tissues or such with neural fibrotic degeneration. siRNAs exhibit a quite superior and specific post-transcriptional suppression effect in vitro; however, in vivo they are rapidly degraded due to serum nuclease activity. Thus, their limited time in vivo becomes problematic, and there is therefore demand for the development of optimized and effective delivery systems. As one example, Ochiya et al. have reported that atelocollagen, a bio-affity material, is a highly suitable siRNA carrier because it has the activity of protecting nucleic acids from nucleases in the body when mixed with the nucleic acids to form a complex (Ochiya, T. et al. Nat. Med. (1999) 5, 707-710; Ochiya, T. et al. Curr. Gene Ther. (2001)1, 31-52). As another example, delivery systems introducing liposomes or viruses directly into the brain have been developed (Xia, H. et al. Nat. Biotechnol. (2002) 20, 1006-1010; Brummelkamp, T. R. et al. Cancer Cell (2002) 2, 243-247; Barton, G. M. and Medzhitov, R. Proc. Natl. Acad. Sci. USA, (2002) 99, 14943-14945; Abbas-Terki, T. et al. Hum. Gene. Ther. (2002) 13, 2197-2201). siRNAs are also being developed for use in gene therapy agents for neural fibrotic degenerative diseases, based on the methods described above and the like. For example, transgenic mice have been used to develop siRNAs against BACE1 (Beta-site APP Cleaving Enzyme: β-secretase) for reducing in vivo amyloid formation. A delivery system in which BACE1 siRNAs are inserted into lentivirus vectors and expressed in the brain has been reported (Singer et al. Nat. Neurosci. (2005) 8(10), 1343-1349). These systems are all examples, and the delivery systems of the present invention are not limited thereto.

The agents of the present invention are administered to mammals including humans at required (effective) doses, within a dose range considered to be safe. Ultimately, the doses of the agents of the present invention can be appropriately determined by medical practitioners or veterinarians after considering the dosage form and administration method, and the patient's age and weight, symptoms, and the like. For example, adenoviruses are administered once a day at a dose of about 106 to 1013 viruses every one to eight weeks, although the doses vary depending on the age, sex, symptoms, administration route, administration frequency, and dosage form.

Commercially available gene transfer kits (for example: AdenoExpress™, Clontech) may be used to introduce siRNAs or shRNAs into target tissues or organs.

When the agents of the present invention are used, the type of disease and site to which the agents are applied are not particularly limited, as long as the disease develops neural fibrotic degeneration; for example, the agents are applied to cerebrospinal neural fibrotic degenerative diseases, peripheral neural fibrotic degenerative diseases, Parkinson's disease, Alzheimer's disease, amyotrophic lateral sclerosis, polyglutamine disease, myelopathic muscular atrophy, Huntington's disease, multiple sclerosis, and such. The above diseases may occur in combination with other diseases.

The present invention also provides methods of screening for neural fibrotic degeneration-suppressing agents, wherein the methods comprise selecting, from test samples, substances with the activity of inhibiting the production or accumulation of chondroitin sulfate proteoglycans. Neural fibrotic degeneration-suppressing agents or candidate compounds for neural fibrotic degeneration-suppressing agents can be efficiently obtained using the screening methods of the present invention.

Preferred embodiments of the screening methods of the present invention are methods of screening for neural fibrotic degeneration-suppressing agents that comprise the step of selecting substances with any of the activities (a) to (d):

(a) promoting the degradation of chondroitin sulfate proteoglycans;

(b) inhibiting the synthesis of chondroitin sulfate proteoglycans;

(c) desulfating chondroitin sulfate proteoglycans; and

(d) inhibiting the sulfation of chondroitin sulfate proteoglycans.

Representative examples based on fundamental principles common in screening for these substances include methods comprising the following procedure: A preferred procedure uses tools (1) to (3) below, and is as follows: (1) and (2) are mixed in a test tube or culture dish, and the resulting effect is simply detected using (3).

(1) chondroitin sulfate proteoglycans (CSPGs) themselves, or glycosaminoglycans (GAG) chains, or cells synthesizing (producing) CSPGs or GAG chains

(2) test compounds (for example, enormous compound libraries owned by pharmaceutical companies)

(3) methods for detecting CSPG cleavage sites, the amount of CSPGs, or the amount of free glycosaminoglycans (GAGs)

Embodiments of the screening methods of the present invention are exemplified below. In the embodiments described below, the chondroitin sulfate proteoglycans, synthetases, compounds suppressing desulfation enzymes, sulfotransferases, degradation-promoting enzymes, and desulfation enzymes to be used include those derived from humans, mice, rats, and others, but are not limited thereto. Chondroitin sulfate proteoglycan portions are components such as glycosaminoglycan chains or core proteins, or portions thereof. The chondroitin sulfate proteoglycan portions are not particularly limited.

The test compounds to be used in the embodiments described below are not particularly limited, but include, for example, single compounds, such as natural compounds, organic compounds, inorganic compounds, proteins, and peptides, as well as compound libraries, expression products of gene libraries, cell extracts, cell culture supernatants, products of fermenting microorganisms, extracts of marine organisms, and plant extracts.

In the embodiments described below, the “contact” with test compounds is typically achieved by mixing the test compounds with chondroitin sulfate proteoglycans, portions thereof, synthetases, compounds suppressing desulfation enzymes, sulfotransferases, degradation-promoting enzymes, or desulfation enzymes, but the “contact” is not limited to this methods. For example, the “contact” can also be achieved by contacting test compounds with cells expressing these proteins or portions thereof.

In the embodiments described below, the “cells” include those derived from humans, mice, rats, and such, but are not limited thereto. Cells of microorganisms, such as Escherichia coli and yeasts, which are transformed to express the proteins used in each embodiment, can also be used. For example, the “cells that express chondroitin sulfate proteoglycans” include cells that express endogenous genes for chondroitin sulfate proteoglycans, and cells that express introduced foreign genes for chondroitin sulfate proteoglycans. Such cells that express foreign genes for chondroitin sulfate proteoglycans can typically be prepared by introducing host cells with expression vectors carrying a chondroitin sulfate proteoglycan gene as an insert. The expression vectors can be prepared using standard genetic engineering techniques.

The “chondroitin sulfate proteoglycan core proteins” described below include, for example, core proteins of matrix-type chondroitin sulfate proteoglycans, such as aggrecan, versican, neurocan, and brevican, and core proteins of membrane chondroitin sulfate proteoglycans, such as decorin, biglycan, fibromodulin, and PG-Lb. The “synthetases” include, for example, GaiNAc4ST-1, GalNAc4ST-2, GALNAC4S-6ST, UA20ST, GalT-I, GalT-II, GlcAT-I, and XylosylT. The “sulfotransferases” include, for example, chondroitin D-N-acetylgalactosamine-4-O-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2. The “degradation-promoting enzymes” include, for example, ADAMTS-1, ADAMTS-4, ADAMTS-5, chondroitinase ABC(ChABC), chondroitinase AC, chondroitinase B, and calpain I. The “desulfation enzymes” include, for example, chondroitin-4-sulfatase and chondroitin-6-sulfatase.

Embodiments of the screening methods of the present invention include methods comprising the step of selecting compounds that have the activity of promoting the degradation of chondroitin sulfate proteoglyeans. An example of the above-mentioned methods of the present invention comprises the steps of:

(a) contacting test compounds with chondroitin sulfate proteoglycans or portions thereof;

(b) measuring the abundance of chondroitin sulfate proteoglycans or portions thereof, and

(c) selecting substances that reduce the abundances as compared with those determined in the absence of the test compounds.

In the above methods, first, test compounds are contacted with chondroitin sulfate proteoglycans or portions thereof.

In these methods, the amount of the chondroitin sulfate proteoglycans or portions thereof is then measured. The measurement can be conducted by methods known to those skilled in the art. For example, the amounts can be detected using labeled compounds or antibodies that bind to the chondroitin sulfate proteoglycans or portions thereof, and then measuring the amount of the label. Alternatively, the detection can be achieved by chromatography or mass spectrometry.

In these methods, compounds that reduce the abundance of the chondroitin sulfate proteoglycans or portions thereof as compared with in the absence of a test compound (the control) are then selected. Compounds resulting in a reduction can be used as therapeutic agents for neural fibrotic degeneration.

Below is a brief illustrative example of the methods able to assess (measure) whether a test compound has the above activity (a): the activity of promoting the degradation of chondroitin sulfate proteoglycans.

Embodiment of the methods for screening for the above activity (a) of promoting the degradation of chondroitin sulfate proteoglycans:

A CS-GAG, such as chondroitin sulfate A (CS-A), CS-B, CS-C (Seikagaku Co., ICN, Sigma, and others), or human-derived proteoglycan (BGN, ISL, and others), is prepared, and 96-well plates are coated with it at a concentration of 10 μg/ml (using known methods, such as in Kawashima H. et al., J. Biol. Chem. 277:12921-12930, 2002). Various test compounds are added to each well of the plates. After two hours of reaction at 37° C., changes in CS-GAG are detected.

Detection methods include, for example, the simple WFA lectin (Wisteria floribunda lectin)-binding method. Since WFA lectin binds to the GalNAc residues of CS-GAG chains, it can easily detect CS-GAGs. Chondroitinase ABC is used as a positive control for test compounds. The principle behind this use of chondroitinase ABC is that its addition degrades CS-GAG chains, making it impossible for WFA lectin to bind them. More specifically, FITC-labeled WFA lectin (EY Co.) is added to the CS-coated wells before and after mixing the test compounds, and changes in the intensity of FITC fluorescence in the wells due to the CS-GAG degradation can be quantified and digitized very simply by using detection devices, such as fluorescence plate readers or fluorescence microscopes. Compounds whose addition most reduces fluorescent values may be determined to be novel therapeutic candidate compounds that fulfill the concept of the present invention.

In an alternative detection method, anti-CS antibody (clone CS56, Seikagaku Co.) can be used to directly label CS-GAGs. As with WFA lectin, large-scale screening can be carried out simply and in very short time by adding FITC-labeled anti-CS antibody to CS-coated wells and examining changes in fluorescence value.

In more specific detection methods, GAG content is accurately quantified and digitized by simply using the plates before and after mixing of test compounds in an sGAG Assay Kit (Wieslab Co.), an ELISA Kit for Sulphanated Glycosaminoglycans (Funakoshi Co.), or such.

More specifically, the reducing ends of free GAG chains can easily be fluorescently labeled by adding 2-aminobenzamide, 2-aminopyridine (2-AB and 2-AP, respectively; LUD Co. and others), or the like to the plates before and after mixing of test compounds, which enables more specific analysis using HPLC, MALDI-MS, LC-MS, or such to determine the types of sugar chains and even the content of each type of chain. These methods, which examine the properties of candidate compounds in detail, take screening to the next level.

Other embodiments of the screening methods of the present invention include methods comprising the step of selecting substances with the activity of inhibiting the synthesis of chondroitin sulfate proteoglycans. These methods of the present invention comprise, for example, the steps of:

(a) contacting test compounds with cells expressing chondroitin sulfate proteoglycans or portions thereof, extracts of these cells, or groups of substances including those enzymes and substrates constituting the process of chondroitin sulfate proteoglycan synthesis;

(b) measuring the amount of synthesized chondroitin sulfate proteoglycans or intermediates thereof in the above-mentioned cells, cell extracts, or group of substances; and

(c) selecting compounds that reduce the amount as compared to in the absence of the test compounds.

In the above methods, test compounds are first contacted with cells expressing chondroitin sulfate proteoglycans or portions thereof, extract of these cells, or groups of substances including those enzymes and substrates that constitute the process of chondroitin sulfate proteoglycan synthesis.

Next, the amount of synthesized chondroitin sulfate proteoglycans or intermediates thereof is measured. The measurement can be performed by those skilled in the art using known methods; for example, methods using labeled antibodies, mass spectrometry, and chromatography can be used.

Further, compounds that reduce (suppress) the synthesized amount as compared with in the absence of the test compounds (the control) are selected. Compounds resulting in a reduction (suppression) can be used as therapeutic agents for neural fibrotic degeneration.

Below is a brief illustrative example of the methods able to assess (measure) whether a test compound has the above activity (b): the activity of inhibiting the synthesis of chondroitin sulfate proteoglycans.

Embodiment of the methods for screening for the above activity (b) of inhibiting the synthesis of chondroitin sulfate proteoglycans:

Cells and cell lines synthesizing chondroitin sulfate are known to researchers in the art. In human, for example, chondroitin sulfate is produced after 16 hours of cell culture by standard methods for culturing mononuclear cells isolated from peripheral blood collected from healthy subjects (Uhlin-Hansen L. et al., Blood 82:2880, 1993; etc.). Alternatively, for more convenience, there are many examples of known cell; for example, the fibroblast cell line NIH3T3 (Phillip H A, et al. J. Biol. Chem. 279:48640, 2004; etc.); the renal tubule-derived cancer cell line ACHN (Kawashima H. et al., J. Biol. Chem. 277:12921, 2002), the renal distal tubule-derived cell line MDCK (Borges F. T. et al., Kidney Int. 68:1630, 2005; etc.), and the vascular endothelial cell line HUVEC (Schick B. P. et al., Blood 97:449, 2001; etc.). Various test compounds are added during the process of culturing such cell lines for set periods, and changes in the amount of CS-GAG before and after culture can be easily evaluated by the above-described method of (a). Compounds that suppress the increase in the amount of CS-GAG after culture (which thus reflects the amount of synthesized CS-GAG ) can be easily determined to be candidate therapeutic compounds that fulfill the concept of the present invention.

As a further option, cell lines constitutively expressing the genes for CS-GAG synthetases such as GalNAc4ST-1 and XylosylT can be prepared by introducing the genes into CHO cells, L cells, or such by well-known methods. The use of such cell lines that constitutively synthesize CS-GAG allows more clear determination of candidates for therapeutic compounds.

In another embodiment, the screening methods of the present invention include methods comprising the step of selecting substances with the activity of desulfating chondroitin sulfate proteoglycans. The above methods of the present invention comprise, for example, the steps of:

(a) contacting test compounds with chondroitin sulfate proteoglycans or portions thereof;

(b) measuring the amount of sulfation in the chondroitin sulfate proteoglycans or portions thereof; and

(c) selecting substances that reduce the amount of sulfation as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the desulfated structures remaining in the chondroitin sulfate proteoglycans or portions thereof, and measuring the amount of the label. Alternatively, the measurement can be achieved by chromatography or mass spectrometry or such.

Then, in the present methods compounds that reduce the abundances of the chondroitin sulfate proteoglycans or portions thereof as compared with in the absence of the test compounds (the control) are selected. Compounds resulting in a reduction can be used as therapeutic agents for neural fibrotic degeneration.

Below is a brief illustrative example of the methods able to assess (measure) whether a test compound has the above activity (c): the activity of desulfating chondroitin sulfate proteoglycans.

Embodiment of the methods for screening for the above activity (c) of desulfating chondroitin sulfate proteoglycans:

By using essentially the same method as described above in (a), human-derived proteoglycans (BGN Co., ISL Co., etc.) or such are prepared and coated on to 6-well plates at a concentration of 10 μg/ml (by known methods, such as those described in Kawashima H. et al., J. Biol. Chem. 277:12921-12930, 2002). Various test compounds are added to each well of the plates, and alterations in CS-GAG are detected after two hours of reaction at 37° C.

In this detection method, desulfated moieties can be easily detected using the reaction of either anti-proteoglycan Δdi4S antibody (clone: 2-B-6, which recognizes sulfated moieties at position 4) or anti-proteoglycan Δdi6S antibody (clone: 3-B-3, which recognizes sulfated moieties at position 6) (both from Seikagaku Co.) with the disaccharide structure of the desulfated fragments that remain in the core protein of proteoglycans after desulfation. Thus, FITC-labeled 2-B-6 or 3-B-3 antibody is reacted in such plates before and after mixed culture, and changes in the fluorescence value can be simply detected. Compounds whose addition increases the fluorescence intensity can be determined to be substances that promote desulfation, and are easily identified as novel candidate therapeutic compounds that fulfill the concept of the present invention.

Another embodiment of the screening methods of present invention includes methods comprising the step of selecting substances with the activity of inhibiting the sulfation of chondroitin sulfate proteoglycans. The above methods of the present invention comprise, for example, the steps of:

(a) contacting test compounds with cells expressing chondroitin sulfate proteoglycans or portions thereof, extracts of these cells, or groups of substances including those enzymes and substrates constituting the process of sulfation of chondroitin sulfate proteoglycans;

(b) measuring the activity of sulfation of chondroitin sulfate proteoglycans in the above-mentioned cells, cell extracts, or groups of substances; and

(c) selecting compounds that reduce the activity as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the sulfated structures of the chondroitin sulfate proteoglycans or portions thereof, and measuring the amount of the label. Alternatively, the measurement can be achieved by chromatography or mass spectrometry and such.

Then, compounds that reduce the abundance of the chondroitin sulfate proteoglycans or portions thereof as compared with in the absence of the test compounds (the control) are selected. Compounds resulting in a reduction can be used as therapeutic agents for neural fibrotic degeneration.

Below is a brief illustrative example of the methods able to assess (measure) whether a test compound has the above activity (d): the activity of inhibiting the sulfation of chondroitin sulfate proteoglycans.

Embodiment of the methods for screening for the above activity (d) of inhibiting the sulfation of chondroitin sulfate proteoglycans:

The cells and cell lines that promote the sulfation of chondroitin sulfates are the same as described above in (c). Various test compounds are mixed during a set period of culture of such cell lines, and the degree of sulfation before and after the culture can be easily determined by, for example, using an antibody that recognizes sulfation at position 4 (clone: LY111) or an antibody that recognizes sulfation at position 6 (clone: MC21C) (both from Seikagaku Co.). Fluorescence values may be compared between before and after the culture by using fluorescently labeled antibodies. Alternatively, the same detection method as described above in (c) can be conducted using 2-B-6 or 3-B-3 antibodies before and after culture. Compounds that suppress an increase in the sulfation after cell culture (an increase in the fluorescence value for LY111 or MC21C), or compounds that promote the progression of desulfation after cell culture (an increase in the fluorescence value for 2-B-6 or 3-B-3) can be easily determined to be candidate therapeutic compounds that fulfill the concept of the present invention.

As a further option, cell lines that constitutively express sulfotransferase genes such as C4ST-1 and C6ST-1 can be prepared by introducing the genes into CHO cells, L cells, or such by well-known methods. The use of such cell lines that constitutively add sulfate groups allows more clear determination of candidates for therapeutic compounds.

Other preferred embodiments of the present invention are methods of screening for neural fibrotic degeneration-suppressing agents in which compounds that reduce the expression level of a gene encoding a CSPG core protein, synthetase, desulfation enzyme-suppressing protein, or sulfotransferase of the present invention, or compounds that increase the expression level of a gene for an enzyme that desulfates CSPGs or promotes the degradation of CSPGs, are selected; wherein the method comprises the steps of:

(a) contacting test compounds with cells expressing a gene encoding a CSPG core protein, synthetase, desulfation enzyme-suppressing protein, sulfotransferase, degradation-promoting enzyme, or desulfating enzyme;
(b) determining the expression level of the gene in the cells;
(c) comparing the expression level with that in the absence of the test compounds (the control); and
(d) selecting compounds that reduce the expression level of the gene of the CSPG core protein, synthetase, desulfation enzyme-suppressing protein, or sulfotransferase as compared with the control, or compounds that increase the expression level of the gene of the CSPG desulfating enzyme or the CSPG degradation-promoting enzyme as compared with the control.

In the above methods, test compounds are first contacted with cells expressing a gene encoding a CSPG core protein, synthetase, desulfating enzyme-suppressing protein, sulfotransferase, degradation-promoting enzyme, or desulfating enzyme.

Next, the expression level of the gene encoding the core protein, synthetase, desulfating enzyme-suppressing protein, sulfotransferase, degradation-promoting enzyme, or desulfating enzyme is measured. Herein, “expression of the gene” includes both transcription and translation. Gene expression level can be measured by methods known to those skilled in the art.

For example, mRNAs are extracted from cells expressing any one of the above-described proteins by conventional methods, and these mRNAs can be used as templates in Northern hybridization, RT-PCR, DNA arrays, or such to measure the transcription level of the gene. Alternatively, protein fractions are collected from cells expressing a gene encoding any of the above-described proteins, and expression of the protein can be detected by electrophoresis such as SDS-PAGE to measure the level of gene translation. Alternatively, the level of gene translation can be measured by detecting the expression of any of the above-described proteins by Western blotting using an antibody against the proteins. Such antibodies for use in detecting the proteins are not particularly limited, as long as they are detectable. For example, both monoclonal and polyclonal antibodies can be used.

Next, the expression level is compared with that in the absence of the test compounds (the control).

Then, when the gene encodes a CSPG core protein, synthetase, desulfating enzyme-suppressing protein, or sulfotransferase, compounds that reduce (suppress) the expression level of the gene as compared with a control are selected. The compounds resulting in a reduction (suppression) can be agents for suppressing neural fibrotic degeneration or candidate compounds for treating neural fibrotic degeneration.

Alternatively, when the gene encodes a CSPG desulfating enzyme or an enzyme promoting CSPG degradation, compounds that increase (enhance) the expression level of the gene as compared with a control are selected. Compounds resulting in an increase (enhancement) can be agents for suppressing neural fibrotic degeneration or candidate compounds for treating neural fibrotic degeneration.

An embodiment of the screening methods the present invention is a method in which compounds that reduce the expression level of a gene encoding a CSPG core protein, synthetase, desulfation enzyme-suppressing protein, or sulfotransferase of the present invention, or compounds that increase the expression level of a gene for a CSPG degradation-promoting enzyme or a CSPG desulfating enzyme, can be selected using the expression of a reporter gene as an indicator. The above methods of the present invention comprise, for example, the steps of:

(a) contacting test compounds with cells or cell extracts containing a DNA structured such that a reporter gene is operably linked to a transcriptional regulatory region of a gene encoding a CSPG core protein, synthetase, desulfating enzyme-suppressing protein, sulfotransferase, degradation-promoting enzyme, or desulfating enzyme;
(b) measuring the expression level of the reporter gene;
(c) comparing the level with the that in the absence of the test compounds (the control); and
(d) selecting compounds that reduce the expression level of the reporter gene as compared with the control when the reporter gene is operably linked with a transcriptional regulatory region of the gene encoding a CSPG core protein, synthetase, desulfating enzyme-suppressing protein, or sulfotransferase; or selecting compounds that increase the expression level of the reporter gene as compared with the control when the reporter gene is operably linked with a transcriptional regulatory region of a gene encoding a CSPG degradation-promoting enzyme or a CSPG desulfating enzyme.

In the above methods, test compounds are first contacted with cells or cell extracts containing DNAs structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme.

Herein, “operably linked” means that a reporter gene is linked with a transcriptional regulatory region of a gene encoding a CSPG core protein, synthetase, desulfating enzyme-suppressing protein, sulfotransferase, degradation-promoting enzyme, or desulfating enzyme, such that expression of the reporter gene is induced upon binding of transcriptional factors to the transcriptional regulatory region. Therefore, the meaning of “operably linked” also includes cases where a reporter gene is linked with a different gene and produces a fusion protein with a different gene product, as long as expression of the fusion protein is induced upon the binding of transcriptional factors to the transcriptional regulatory region of the gene encoding the CSPG core protein, synthetase, desulfating enzyme-suppressing protein, sulfotransferase, degradation-promoting enzyme, or desulfating enzyme. Those skilled in the art can obtain the transcriptional regulatory regions of genes encoding CSPG core proteins, synthetases, desulfating enzyme-suppressing proteins, sulfotransferases, degradation-promoting enzymes, or desulfating enzymes that are present in the genome, based on the cDNA nucleotide sequences of the genes encoding the CSPG core proteins, synthetases, desulfating enzyme-suppressing proteins, sulfotransferases, degradation-promoting enzymes, or desulfating enzymes.

The reporter genes for use in these methods are not particularly limited, as long as their expression is detectable. The reporter genes include, for example, the CAT gene, the lacZ gene, the luciferase gene, and the GFP gene. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, cells introduced with vectors carrying such structures as inserts. Such vectors can be prepared by methods well known to those skilled in the art. The vectors can be introduced into cells by standard methods, for example, calcium phosphate precipitation, electroporation, lipofection, and microinjection.

The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include cells in which the structure has been integrated into the chromosomes. A DNA structure can be integrated into chromosomes by methods generally used by those skilled in the art, for example, gene transfer methods using homologous recombination.

The “cell extracts containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, mixtures of cell extracts included in commercially available in vitro transcription-translation kits and DNAs structured such that a reporter gene is operably linked with the transcriptional regulatory region of the gene encoding a CSPG core protein, synthetase, desulfating enzyme-suppressing protein, or sulfotransferase.

“Contact” can be achieved by adding test compounds to a culture medium of “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme”, or by adding test compounds to the above-described commercially available cell extracts containing the DNAs. When the test compound is a protein, contact may also be achieved, for example, by introducing a DNA vector expressing the protein into the cells.

In the above methods, the expression level of the reporter gene is then measured. The expression level of the reporter gene can be measured by methods known to those skilled in the art, depending on the type of the reporter gene. When the reporter gene is the CAT gene, its expression can be determined, for example, by detecting the acetylation of chloramphenicol by the gene product. When the reporter gene is the lacZ gene, its expression level can be determined by detecting the color development of chromogenic compounds due to the catalytic action of the gene expression product. Alternatively, when the reporter gene is the luciferase gene, its expression level can be determined by detecting the fluorescence of fluorogenic compounds due to the catalytic action of the gene expression product. Furthermore, when the reporter gene is the GFP gene, its expression level can be determined by detecting the fluorescence of the GFP protein.

In the above methods, the expression level of the reporter gene is then compared with that in the absence of the test compounds (the control).

In the present methods, compounds that reduce (suppress) the expression level of a reporter gene as compared with a control are then selected, where the reporter gene is operably linked with a gene encoding a CSPG core protein, synthetase, desulfating enzyme-suppressing protein, or sulfotransferase. Compounds resulting in a reduction (suppression) can be agents for suppressing neural fibrotic degeneration or candidate compounds for treating neural fibrotic degeneration.

Alternatively, when the reporter gene is operably linked with a gene encoding a CSPG degradation-promoting enzyme or CSPG desulfating enzyme, compounds that increase (enhance) the reporter gene expression level as compared with a control are selected. Compounds resulting in an increase (enhancement) can be agents for suppressing neural fibrotic degeneration or candidate compounds for treating neural fibrotic degeneration.

The neural fibrotic degeneration-suppressing agents that are found by the screening methods of the present invention are preferably therapeutic or preventive agents for neural fibrotic degenerative diseases.

The present invention also provides kits comprising various agents, reagents, and the like, which are used to conduct the screening methods of the present invention.

The kits of the present invention can be prepared, for example, by selecting adequate reagents from the above-described various reagents, depending on the screening method to be conducted. The kits of the present invention may contain, for example, the chondroitin sulfate proteoglycans of the present invention. The kits of the present invention may further contain various reagents, vessels, and the like to be used in the methods of the present invention. The kits may appropriately contain, for example, anti-chondroitin sulfate proteoglycan antibodies, probes, various reaction reagents, cells, culture media, control samples, buffers, and instruction manuals containing a description of how to use the kits.

Preferred embodiments of the present invention are the methods of screening for neural fibrotic degeneration-suppressing agents, comprising the step of detecting whether the production or accumulation of chondroitin sulfate proteoglycans is inhibited. Thus, the kits for screening for the neural fibrotic degeneration-suppressing agents of the present invention may contain, for example, oligonucleotides such as probes for the genes encoding CSPG core proteins, and primers to amplify certain regions of these genes; and antibodies recognizing CSPGs (anti-chondroitin sulfate proteoglycan antibodies), which can be used to detect chondroitin sulfate proteoglycans.

The above-described oligonucleotides specifically hybridize to, for example, DNAs of the genes encoding the versican core protein of the present invention. Herein, “specifically hybridize to” means that the oligonucleotides do not significantly cross-hybridize to DNAs encoding other proteins under standard hybridization conditions, and preferably under stringent hybridization conditions (for example, the conditions described in Sambrook J. et al. “Molecular Cloning” 2nd Ed., Cold Spring Harbour Laboratory Press, New York, USA (1989)). The oligonucleotides are not necessarily perfectly complementary to the nucleotide sequences of the versican core protein genes of the present invention, as long as they allow specific hybridization.

The hybridization conditions in the present invention include, for example, conditions such as “2×SSC, 0.1% SDS, and 50° C.”, “2×SSC, 0.1% SDS, and 42° C.”, and “1×SSC, 0.1% SDS, and 37° C.”, and more stringent conditions such as “2×SSC, 0.1% SDS, and 65° C.”, “0.5×SSC, 0.1% SDS, and 42° C.”, and “0.2×SSC, 0.1% SDS, and 65° C.”. More specifically, for methods using Rapid-hyb buffer (Amersham Life Science), prehybridization is carried out at 68° C. for 30 minutes or more; then a probe is added, and after one hour or more of hybrid formation at 68° C., washing is carried out three times with 2×SSC/0.1% SDS at room temperature for 20 minutes, then three times with 1×SSC/0.1% SDS at 37° C. for 20 minutes, and finally twice with 1×SSC/0.1% SDS at 50° C. for 20 minutes. Alternatively, for example, prehybridization is carried out in Expresshyb Hybridization Solution (CLONTECH) at 55° C. for 30 minutes, and then a labeled probe is added thereto, and after one hour or more of incubation at 37-55° C., washing is carried out three times in 2×SSC/0.1% SDS at room temperature for 20 minutes and then once in 1×SSC/0.1% SDS at 37° C. for 20 minutes. More stringent conditions can be achieved, for example, by increasing the temperature for prehybridization, hybridization, or second washing. For example, temperatures for prehybridization and hybridization can be 60° C., and more stringent conditions can be achieved by increasing the temperature to 68° C. In addition to conditions such as the salt concentration of the buffers and the temperature, those skilled in the art can also determine conditions using other conditions including the nucleotide sequence composition of the probe, the probe length and concentration, and reaction time.

The oligonucleotides can be used as probes and primers in the above-described screening kits of the present invention. When the oligonucleotides are used as primers, they are typically 15 to 100 bp long, and preferably 17 to 30 bp long. Such primers are, for example, those of SEQ ID NO: 69 or 70, but they are not particularly limited as long as they can amplify at least a portion of a DNA of the above-described genes of the present invention.

The present invention also provides therapeutic or preventive methods for neural fibrotic degenerative diseases, which comprise the step of administering the agents of the present invention to individuals (for example, to patients and such).

The individuals subjected to the therapeutic or preventive methods of the present invention are not particularly limited, as long as they are organisms that can develop a neural fibrotic degenerative disease; however, humans are preferred.

In general, administration to individuals can be achieved, for example, by methods known to those skilled in the art, such as intraarterial injections, intravenous injections, and subcutaneous injections. The administered dose varies depending on the patient's weight and age, and the administration method or such; however, those skilled in the art (medical practitioners, veterinarians, pharmacists, and the like) can appropriately select a suitable dose.

The present invention also relates to the uses of agents of the present invention in producing neural fibrotic degeneration-suppressing agents.

All prior-art documents cited herein are incorporated by reference herein.

### EXAMPLES

Herein below, the present invention will be specifically described with reference to Examples, but the technical scope of the present invention is not to be construed as being limited thereto.

### Example 1

### Examination of the Expression of the GalNAc4ST-1 and GALNAC4S-6ST Genes After Treatment with GalNAcST (siRNA) or Chondroitinase ABC in MPTP-induced C57BL/6JcL Parkinson's Disease Model Mice

In this example, a mouse model for Parkinson's disease was created by selectively degenerating dop amine neurons using 1-methyl-4-phenyl-1,2,3,6 tetrahydropyridine (MPTP)(Amende et al. (2005) Journal of NeuroEngineering and Rehabilitation 2(20), 1-13); a GalNAcST siRNA agent or chondroitinase ABC agent was administered to these mice, and gene expression and tissue appearance after the treatment were examined and compared.

14-day pregnant C57BL/6JcL mice (CLEA Japan Inc.) were fed and allowed to deliver. Each of the 8-week-old female C57BL/6JcL mice (CLEA Japan Inc.) were intraperitoneally administered with 100 μl of 4 U/ml chondroitinase ABC (Sigma Aldrich Japan) or 200 μl of a mixture consisting of 1 μg of GalNAcST siRNA (a mixture of cocktail sequences of GalNAc4ST-1, GalNAc4ST-2, and GALNAC4S-6ST; GeneWorld) and 1% atelocollagen (Koken), which is an siRNA vehicle. Then, on Days 2, 3, and 4 the mice were administered with MPTP (Sigma Aldrich Japan), which selectively destroys only dopamine neurons, at a dose of 30 mg/kg, three times in total. The mice were then fed, and on Day 8 of the experiment 100 μl of 5 mg/ml BrdU (ZyMED Laboratory Inc.) was administered into their caudal vein. After one hour, the mice were dissected and their brains were excised to prepare samples for immunostaining and gene expression analysis. 1 ml of RNA-Bee (Tel-Test Inc.) was added to every 50 mg of excised organ (brains), which where then crushed in an electric homogenizer (Digital Homogenizer, Asone). Then, 200 μl of chloroform (Sigma Aldrich Japan) was added to each sample. After mixing gently, the samples were cooled on ice for about five minutes, then centrifuged using a centrifuge (Centrifuge 5417R, Eppendorf) at 12,000 rpm and 4° C. for 15 minutes. After centrifugation, 500 μl of the supernatants were transferred to different eppendorf tubes, and an equal volume (500 μl) of isopropanol (Sigma Aldrich Japan) was added to each tube. After mixing, 1 μl of glycogen (Invitrogen) was added to the tubes, which where then cooled on ice for 15 minutes. After cooling on ice for 15 minutes, the mixtures were centrifuged at 12,000 rpm and 4° C. for 15 minutes. Then, the RNA precipitates were washed three times with 1000 μl of 75% ethanol (Sigma Aldrich Japan), air-dried for 30 minutes to one hour, and dissolved in 50 μl of Otsuka distilled water (Otsuka Pharmaceutical). The RNAs were then diluted 100 times with Otsuka distilled water (Otsuka Pharmaceutical) and the concentrations of extracted RNA in the samples were determined using UV plates (Corning Costar) in a plate reader (PowerWave XS, BioTek Inc.).

An RT reaction (cDNA synthesis) was then conducted by the following procedure: the determined concentrations of the yielded RNA samples were adjusted to 500 ng/20 μl. The samples were then heated at 68° C. for three minutes in a block incubator (Astec), and then cooled on ice for ten minutes. After cooling, 80 μl of previously prepared RT PreMix solution (composition: 18.64 μl of 25 mM MgCl₂ (Invitrogen), 20 μl of 5× buffer (Invitrogen), 6.6 μl of 0.1 M DTT (Invitrogen), 10 μl of 10 mM dNTP mix (Invitrogen), 2 μl of RNase Inhibitor (Invitrogen), 1.2 μl of MMLV reverse transcriptase (Invitrogen), 2 μl of random primer (Invitrogen), and 19.56 μl of sterile distilled water (Otsuka distilled water, Otsuka Pharmaceutical)) was added to the samples. The resulting mixtures were incubated in a block incubator (Astec) at 42° C. for one hour. After one hour, the reaction mixtures were heated in a block incubator (Astec) at 99° C. for five minutes, and then cooled on ice. Desired cDNAs were prepared in 100 μl and PCR was carried out using the synthesized cDNAs in the following composition:

1 μl of the cDNAs obtained as described above was combined with 2 μl of PCR buffer (composition: 166 mM (NH₄)₂SO₄ (Sigma Aldrich Japan), 670 mM Tris-HCl (pH 8.8) (Invitrogen), 67 mM MgCl₂.6H₂O) (Sigma Aldrich Japan), and 100 mM 2-mercaptoethanol (Wako)), 0.8 μl of 25 mM dNTP mix (Invitrogen), 0.6 μl of DMSO (Sigma Aldrich Japan), 0.2 μl of primer forward (GeneWorld), 0.2 μl of Primer Reverse (GeneWorld), 15.7 μl of Otsuka distilled water (Otsuka Pharmaceutical), and 0.1 μl of Taq polymerase (Perkin Elmer). The resulting mixtures were reacted in an Authorized Thermal Cycler (Eppendorf) with 30 cycles of 94° C. for 45 seconds, 55° C. for 45 seconds, and 72° C. for 60 seconds. After the reaction, 2 μl of loading dye (Invitrogen) was added to the yielded PCR products. 1.5% of UltraPure Agarose (Invitrogen) gel was prepared, and the PCR products were electrophoresed using Mupid-2 plus (Advance) at 100 V for 20 minutes. After the electrophoresis, the gel was shaken for 20 minutes in a staining solution of ethidium bromide (Invitrogen) 10000-times diluted with 1×LoTE (composition: 3 mM Tris-HCl (pH7.5) (Invitrogen) and 0.2 mM EDTA (pH 7.5) (Sigma Aldrich Japan)). The gel was then photographed using an Exilim (Casio) installed in I-Scope WD (Advance) to observe gene expression.

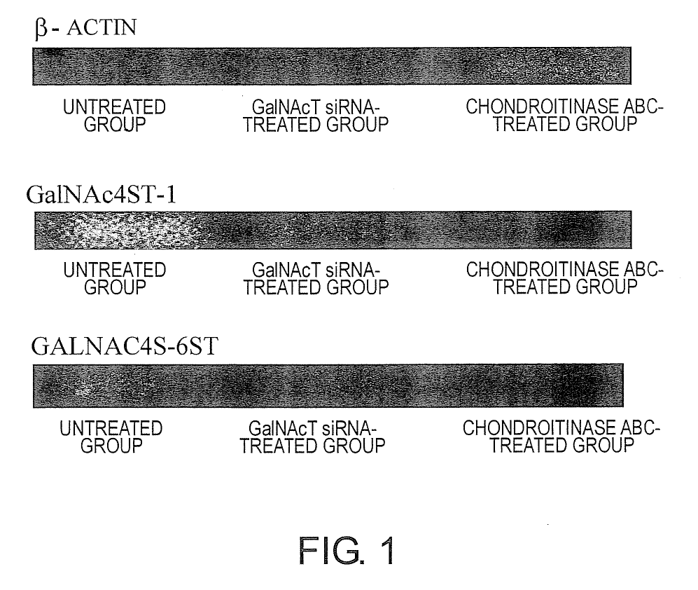

FIG. 1 shows the RT-PCR results for the expression of the GalNAc4ST-1, GALNAC4S-6ST, and β-actin genes in the untreated group and in the GalNAcST siRNA-treated and chondroitinase ABC-treated groups. The GalNAcST siRNA (a mixed siRNA consisting of a GalNAc4ST-1 siRNA cocktail, GalNAc4ST-2 siRNA cocktail, and GALNAC4S-6ST siRNA cocktail; GeneWorld) and primers (Forward and Reverse; Hokkaido System Science) used in this experiment are listed below.

### [Primer Sequences]

*β-actin (GeneWorld)

| | | |
|---|---|---|
| Forward: | 5′-GACCCAGATCATGTTTGAGAC-3′ | (SEQ ID NO:67) |
| Reverse: | 5′-ATGCCTGGGTACATGGTGGTA-3′ | (SEQ ID NO:68) |

### *GalNAc4ST-1 (Hokkaido System Science)

| | | |
|---|---|---|
| Forward: | 5′-ACGTGCCTTTTACACCCAAG-3′ | (SEQ ID NO:69) |
| Reverse: | 5′-GTGTGCCCTTTTCTGTGGAT-3′ | (SEQ ID NO:70) |

[GalNAc4ST-1 siRNA cocktail sequences] (GenBank accession number NM_{—}175140) (GeneWorld)

| | |
|---|---|
| 5′-ACCCCCAACTCGGAACGATGCGGCT-3′ | (SEQ ID NO:71) |
| 5′-TGCATGTTCTCGTCCATCCTGCTG-3′ | (SEQ ID NO:72) |
| 5′-CGCCACCGTGTACTGTACTGTGAAGT-3′ | (SEQ ID NO:73) |
| 5′-AGGCT GCTCCAACTG GAAGAGGGTG-3′ | (SEQ ID NO:74) |

[GalNAc4ST-2 siRNA cocktail sequences] (GenBank accession number NM_{—}199055) (GeneWorld)

| | |
|---|---|
| 5′-ATATAGTATCTAGGATATATGTAG-3′ | (SEQ ID NO:75) |
| 5′-GAAGTACCAAAAGCTGGCTGCTCTA-3′ | (SEQ ID NO:76) |
| 5′-TTCTATCACTTGGACTATTTGATGTT-3′ | (SEQ ID NO:77) |
| 5′-TACACAACTCCACATTTGTAATTTG-3′ | (SEQ ID NO:78) |

[GALNAC4S-6ST siRNA cocktail sequences] (GenBank accession number NM_{—}029935) (GeneWorld)

| | |
|---|---|
| 5′-CCAGAAGCCAAGCTCATTGTTATG-3′ | (SEQ ID NO:79) |
| 5′-CTGTGGAGAGGTTGTACTCAGACTA-3′ | (SEQ ID NO:80) |
| 5′-ATTTGCCTGGAAGACAACGTGAGAGC-3′ | (SEQ ID NO:8l) |
| 5′-GTCCCTTCTGCAGAAGCTGGGCCCACT-3′ | (SEQ ID NO:82) |

According to the RT-PCR results shown in FIG. 1, β-actin as an endogenous control was found to be expressed at the same level in the untreated group, GaiNAcST (siRNA)-treated group, and chondroitinase ABC-treated group. Meanwhile, the expressions of GalNAc4ST-1 and GALNAC4S-6ST were found to be decreased in the GalNAcST (siRNA)-treated group as compared with the untreated group. Thus, it was demonstrated that the gene expression was suppressed (reduced) upon administration of GalNAcST siRNA combined with atelocollagen medium.

### Example 2

### Comparative Examination of Intracerebral CSPG Deposition-Suppressing Effect of Chondroitinase ABC and GalNAcST siRNA Treatments in the MPTP-Induced C57BL/6JcL Parkinson's Disease Model Mice

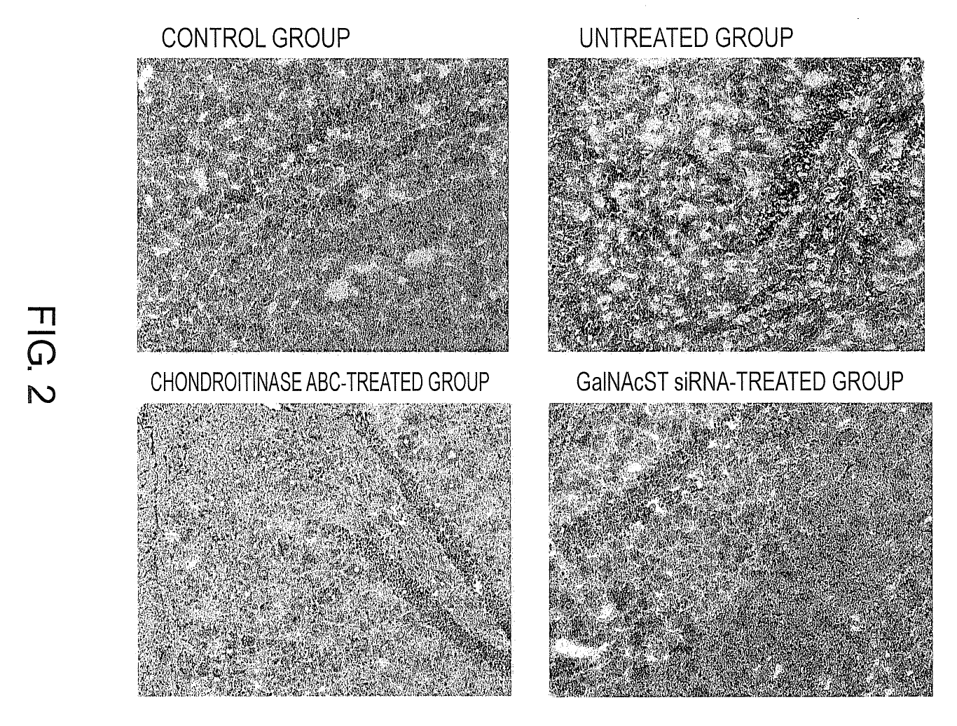

In this Example, the CSPG deposition-suppressing effect was examined and compared using brain tissue samples from Parkinson's disease model mice. The brain tissues obtained in Example 1 were embedded in the freezing embedding medium OCT compound (Miles Lab.), and placed in liquid nitrogen to prepare frozen blocks. The frozen blocks were sliced into 10-μm sections using a cryostat (Microm). The resulting sections were fixed in acetone (Sigma Aldrich Japan) for ten minutes, and then washed with phosphate buffer. An anti-chondroitin sulfate proteoglycans (CSPG) antibody (clone CS56, mouse monoclonal antibody, 10 μg/ml; Seikagaku) was added as the primary antibody, and the sections were reacted at room temperature for one hour. Then, the secondary antibody reaction was conducted using a Histofinee Mouse Stain Kit (Nichirei; used for mouse monoclonal antibodies), and DAB substrate (Nichirei) was added thereto for the enzymatic color reaction. The samples were observed under a light microscope (Leica Microsystems). The histological images are shown in FIG. 2 (the original images are in color). The positive signals in the untreated group showed stronger CSPG accumulation in the area of dentate gyrus than in the control group. It was also found that the accumulation of CSPGs was suppressed in the GalNAcST siRNA-treated and chondroitinase ABC-treated groups. The findings described above demonstrate that in vivo administration of GalNAcST siRNA and chondroitinase ABC suppresses CSPG deposition in the brain tissues of the mouse model for MPTP-induced Parkinson's disease.

### Example 3

### Comparative Examination of the Effect of Chondroitinase ABC and GalNAcST siRNA Treatments in Suppressing Inflammation Associated with Macrophage Infiltration in MPTP-induced C57BL/6JcL Parkinson's Disease Model Mice

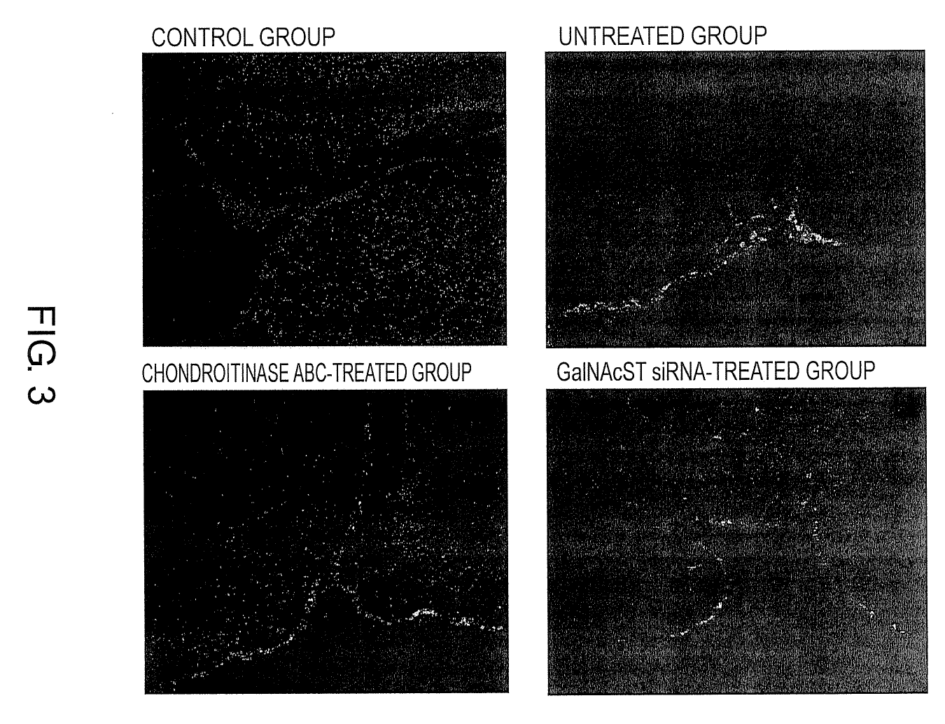

As shown in Example 2, CSPG deposits are known to absorb chemokines, which are in vivo substances that induce inflammatory cells such as macrophages. Since it was hypothesized that CSPG deposition attracted inflammatory cells and thereby resulted in the induction of brain tissue destruction, the effect of GalNAcST administration and chondroitinase ABC on the intracerebral macrophage accumulation was also compared using the same brain samples as described in Example 2. Sections prepared by the same method as described in Example 2 were fixed with 4% paraformaldehyde (PFA)-phosphate buffer (Nacalai Tesque) for ten minutes, and then washed with deionized water. A rat anti-mouse macrophage antibody (clone F4/80, at 1:200 dilution; BMA) was added as the primary antibody, and the sections were incubated at 4° C. overnight. Then, an Alexa488-labeled goat anti-rat IgG antibody (at a 1:200 dilution; Invitrogen) was added as the secondary antibody, and the sections were incubated at room temperature for 30 minutes. Histological images obtained by the procedure described above are shown in FIG. 3 (the original images are in color). The strong positive signals in the untreated group were a result of the accumulation of more macrophages at the periphery of the tissue than in the control group. Furthermore, a comparison of the staining results showed that the accumulation in the GalNAcST siRNA-treated and chondroitinase ABC-treated groups was almost comparable to that in the control group. The results described above show that the accumulation of macrophages in the brain tissues of the Parkinson's disease model mice was significantly suppressed by in vivo administration of GalNAcST siRNA and chondroitinase ABC (accumulated macrophages are shown in green, and nuclei are shown in red using Sytox-Orange (Invitrogen)).

### Example 4

### Comparative Examination of Fibroblast Infiltration-Inhibiting Effect of GalNAcST siRNA and Chondroitinase ABC Treatments in MPTP-induced C57BL/6JcL Parkinson's Disease Model Mice

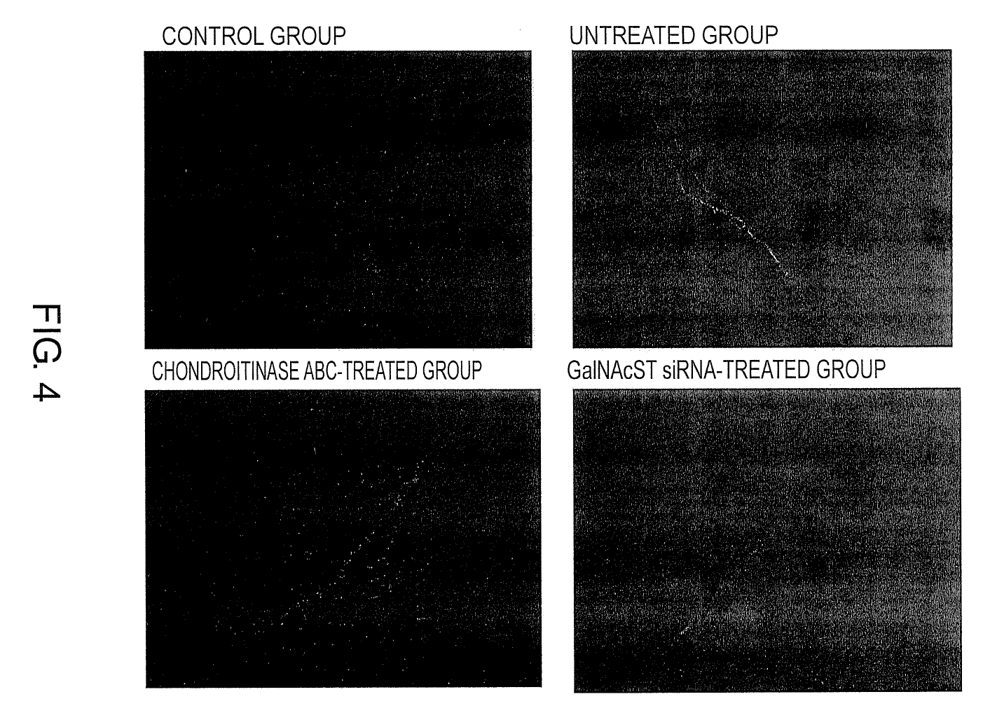

Since there was the possibility that the deposition of CSPGs shown in Example 2 also enhanced cellular fibrosis, brain tissue samples were used as in Examples 2 and 3 to compare histological findings regarding the fibrosis of intracerebral nerve cells and resulting from GalNAcST administration and in vivo chondroitinase ABC administration. Sections prepared by the same method as described in Examples 2 and 3 were fixed with 4% PFA-phosphate buffer (Nacalai Tesque) for ten minutes, and washed with deionized water. An anti-fibroblast antibody (ER-TR7; at 1:100 dilution; BMA) was added as the primary antibody, and the sections were reacted at 4° C. overnight. Then, an Alexa488-labeled goat anti-rat IgG antibody (at 1:200 dilution; Invitrogen) was added as the secondary antibody, and the sections were reacted at room temperature for 30 minutes. Histological images obtained by the procedure described above are shown in FIG. 4 (the original images are in color). The strong positive signals in the untreated group were a result of the intracerebral infiltration of more fibroblasts near the granular cortex in the posterior splenium of the corpus callosum as compared with the control group. A further comparison of the findings revealed that, like the control group, positive findings for fibroblasts are not observed in the GalNAcST siRNA-treated and chondroitinase ABC-treated groups. The results described above demonstrate that in vivo administration of GaiNAcST siRNA or chondroitinase ABC significantly suppresses the positive ER-TR7 signals induced in the brain tissues of Parkinson's disease model mice. Furthermore, in consideration of the CSPG depositions found in this Example and Examples 2 and 3 and the infiltration of macrophages induced by these depositions, the suppression of CSPG overexpression was able to inhibit cellular fibrosis and the induction of inflammatory cells. It can be concluded that this can lead to the suppression of neural fibrotic degeneration (fibroblasts are shown in green, and nuclei are in red using Sytox-Orange (Invitrogen)).

### Example 5

### Comparative Examination of Activation Effect of GalNAcST (siRNA) and Chondroitinase ABC Treatments on Astrocytes in MPTP-induced C57BL/6JcL Parkinson's Disease Model Mice

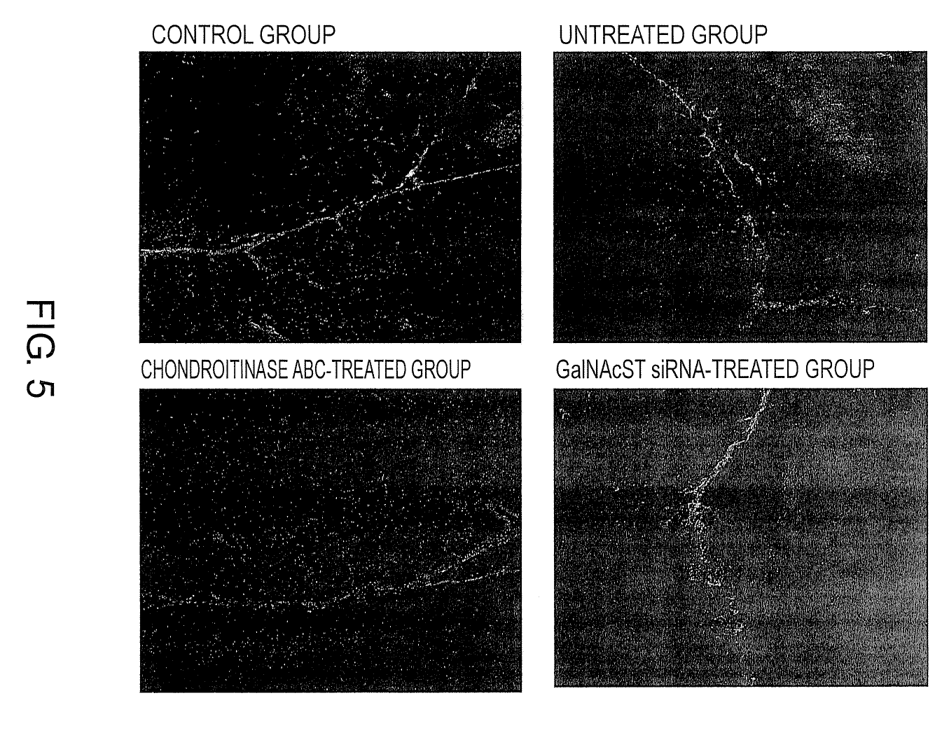

In addition to its many nerve cells, the brain contains glial cells, which are cells that supply nutrients. In this Example, glial cells called astrocytes were stained using an anti-GFAP antibody, and histological findings were compared after GalNAcST administration or in vivo chondroitinase ABC administration. Sections obtained by the same procedure as described in Examples 2, 3, and 4 were fixed with 4% PFA-phosphate buffer (Nacalai Tesque) for ten minutes, and then washed with deionized water. An anti-GFAP antibody (at 1:20 dilution; Santa Cruz Biotechnology) was added as the primary antibody, and the sections were reacted at 4° C. overnight. Then, an Alexa488-labeled donkey anti-goat IgG antibody (at 1:20 dilution; Invitrogen) was added as the secondary antibody, and the sections were reacted at room temperature for 30 minutes. FIG. 5 shows histological images for the control group, untreated group, GalNAcST siRNA-treated group and chondroitinase ABC-treated group (the original images are in color). In the control group, astrocytes were found normally in the granular cortex in the posterior splenium of the corpus callosum; however, this was not the case for the untreated group. This finding suggests that MPTP selectively destroyed astrocytes from among the glial cells. Meanwhile, the signals in the GalNAcST siRNA-treated and chondroitinase ABC-treated groups were found to be stronger than that in the untreated group. Astrocytes are glial cells known chiefly for their role in maintaining the function of nerve cells. The above findings suggest that the suppression of CSPGs restored the function of glial cells called astrocytes and improved nerve cell function (astrocytes are shown in green, and nuclei are in red using Sytox-Orange (Invitrogen)).

### Example 6

### Comparative Examination of Activation Effect of GalNAcST (siRNA) Treatment on Dopamine Neurons in MPTP-induced C57BL/6JcL Parkinson's Disease Model Mice

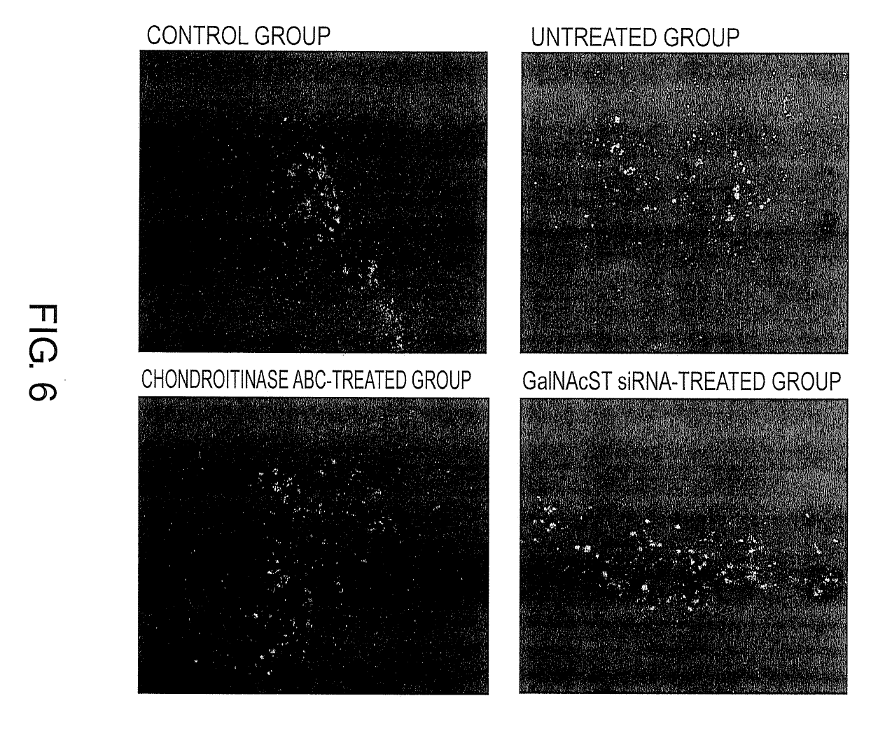

To ultimately elucidate the results shown in the above Examples, the prepared tissue section samples were stained for dopamine neurons with an antibody against tyrosine hydroxylase, a marker for dopamine neurons, and then histological features were compared. Tyrosine hydroxylase (TH) is an enzyme that converts the dopamine precursor into dopamine. Sections prepared by the same procedure as described in Examples 2, 3, and 4 were fixed with 4% PFA-phosphate buffer (Nacalai Tesque) for ten minutes, and then washed with deionized water. A rabbit anti-tyrosine hydroxylase polyclonal antibody (at 1:50 dilution; Calbiochem) was added as the primary antibody, and the sections were reacted at room temperature for one hour. Then, an Alexa488-labeled donkey anti-rabbit antibody (at 1:200 dilution; Invitrogen) was added as the secondary antibody, and the sections were reacted at room temperature for 30 minutes. FIG. 6 shows histological images for the control group, untreated group, GalNAcST siRNA-treated group, and chondroitinase ABC-treated group (the original images are in color). In the control group, the expression of tyrosine hydroxylase was observed to be normal near the superior colliculi of the midbrain; however, the untreated group showed a negative signal. This finding suggests that MPTP selectively destroyed dopamine neurons. However, the signals were found to be stronger in the GalNAcST siRNA-treated and chondroitinase ABC-treated groups than in the untreated group. Thus, it was concluded that the function of dopamine neurons was expected to be restored through the activation of astrocytes by in vivo administration of GalNAcST siRNA or chondroitinase ABC, as described in Example 5 (TH is shown in green, and nuclei are shown in red using Sytox-Orange (Invitrogen)).

### INDUSTRIAL APPLICABILITY

In the present invention, the influence of chondroitin sulfate proteoglycan (CSPG) accumulation was examined; for example, siRNAs against N-acetylgalactosamine-4-O-sulfotransferases (N-acetylgalactosamine-4-O-sulfotransferase-1, N-acetylgalactosamine-4-O-sulfotransferase-2, and N-acetylgactosamine-4-sulfate 6-O-sulfotransferase (GalNAc4ST-1, GalNAc4ST-2, and GALNAC4S-6ST, respectively), which transfer sulfate groups of acetylgalactosamine, a CSPG side chain, and chondroitinase ABC, an enzyme that degrades acetylgalactosamine, also a CSPG side chain, suppress the death of dopamine neurons by suppressing the sulfation of CSPG GAG chains and the accumulation of CSPGs in the hypothalamus of the brain, and thereby produce a therapeutic or preventive effect on Parkinson's disease. Further, according to the present invention, the overexpression and accumulation of CSPGs is also thought to be a factor in the impairment of brain function in neural fibrotic degenerative diseases, such as Alzheimer's disease, polyglutamine disease, amyotrophic lateral sclerosis, myelopathic muscular atrophy, Huntington's disease, and multiple sclerosis, which are thought to develop due to an accumulation of abnormal proteins. The neural fibrotic degeneration-suppressing agents of the present invention can effectively improve the pathological condition through novel action mechanisms and pharmaceutical regimens, and can thus further improve QOL for patients and provide superior therapeutic methods that are useful in practicing medicine.

All the publications, patents, and patent applications cited herein are incorporated by reference herein in their entirety.

1. A neural fibrotic degeneration-suppressing agent comprising as an active ingredient a substance that inhibits the production or accumulation of a chondroitin sulfate proteoglycan.

2. The agent of claim 1, wherein the substance has an activity of promoting the degradation of a chondroitin sulfate proteoglycan.

3. The agent of claim 1, wherein the substance has an activity of inhibiting the synthesis of a chondroitin sulfate proteoglycan.

4. The agent of claim 1, wherein the substance has an activity of desulfating a chondroitin sulfate proteoglycan.

5. The agent of claim 1, wherein the substance has an activity of inhibiting the sulfation of a chondroitin sulfate proteoglycan.

6. The agent of claim 1, wherein the production or accumulation of a chondroitin sulfate proteoglycan is inhibited in a brain.

7. The agent of claim 1, which is used for treating or preventing a neural fibrotic degenerative disease.

8. The agent of claim 7, wherein the neural fibrotic degenerative disease is a cerebrospinal or peripheral neural fibrotic degenerative disease.

9. The agent of claim 7, wherein the neural fibrotic degenerative disease is Parkinson's disease, Alzheimer's disease, amyotrophic lateral sclerosis, polyglutamine disease, myelopathic muscular atrophy, Huntington's disease, or multiple sclerosis.

10. A method of screening for a neural fibrotic degeneration-suppressing agent, which comprises selecting from a test sample a substance with an activity of inhibiting the production or accumulation of a chondroitin sulfate proteoglycan.

11. The method of claim 10, which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.

12. The method of claim 10, wherein the neural fibrotic degeneration-suppressing agent is used for treating or preventing a neural fibrotic degenerative disease.

13. A method of treating or preventing a neural fibrotic degenerative disease, which comprises administering to a patient in need thereof a substance that inhibits the production or accumulation of a chondroitin sulfate proteoglycan.


SEQUENCE LISTING (83 sequences)

SEQ ID NO: 1 - DNA, 7382 bp, Mus musculus
  CDS (133)..(6531)
ccccccgcgcagagcatccctgctgcagcgcagcaagacccggggctggcagccccaggaatccctagctgcttcgcagggataaaggactgaagttcttggaggagcgagtccaactcttcaagctgaactatgaccactttactcttggtctttgtgactctgagggtcatcgctgcagtgatctcagaagaagttccagaccatgacaactcactgagcgtgagcatccctcaaccatccccattgaaggtcctcctagggtcttccctcaccatcccctgctacttcatcgaccccatgcatcctgtgaccactgccccctccactgcccccctcaccccaagaatcaagtggagccgtgtttccaaggaaaaggaggtggtactgctggtggccactgaaggacaggttcgagtcaacagcatctaccaagacaaggtgtcgctccccaactatccagccatccccagcgatgctaccttggagatccagaaccttcgctccaatgactctgggatctaccgctgtgaagtgatgcacggcatcgaggacagcgaagccaccctggaggtcatagtgaaaggtattgtgttccactacagagctatttccacacgctacaccctggactttgatcgagcacagcgggcttgcctacagaacagcgccatcatcgccaccccagaacaactgcaggctgcctatgaggatggcttccaccagtgcgatgcaggctggctggctgaccagacagtcagataccccatccacacgccccgggaaggttgctatggtgacaaggacgagttccctggagtgagaacctacggaatccgggacaccaatgagacctatgatgtgtactgcttcgctgaggagatggagggtgaggtcttttatgcgacatccccagagaaattcaccttccaggaggcagccaacgagtgccggaggctgggggcacgcttggccaccacaggccagctctacctggcctggcagggcggtatggacatgtgcagcgctggctggctggcggaccgcagcgtgcgctaccccatctccaaggctcggcccaactgcgggggcaacctcctgggtgtaaggactgtctatctacacgccaaccagacaggctatcctgatccctcatcccgatatgacgccatctgttacacaggtgaagattttgtagacatcccagaaaacttcttcggggtgggtggtgaagacgacatcaccatccagacggtgacctggccagacctggagctgcccttgccccgtaatgtcacagagggagaggccctgggcagcgtgatcctcacggcaaagcccatctttgacctgtcccccactatctcagagccgggggaggccctcacacttgcccctgaagtggggagcacagccttcccagaggctgaggagagaactggagaagccaccagaccctggggctttcctgcagaagtcacacgtgggccggactctgccactgcctttgccagtgaggacctggtagtgcgagtgaccatctctccaggtgcagctgaagtccctggtcagccccacttgccagggggagttgtattccactatcgcccaggctccaccagatactctctgacatttgaggaggcacagcaggcctgcatgcacaccggggccatcattgcctctcctgagcagctccaagctgcctatgaggcaggctatgagcagtgtgatgctggctggctgcaggaccagaccgtcagataccccattgtgagcccacgaaccccatgtgtgggtgacaaagacagcagcccaggagtcaggacctaccgcgtgcgcccatcatcagaaacctatgatgtctactgctacgtggacaagcttgagggggaagtgttcttcgccacacgcctcgagcagttcaccttccaggaagcgcgggccttctgtgcggctcaaaatgccaccctggcctccaccggccagctctatgctgcctggagccagggtctggacaagtgctatgctggctggctggcagatggcaccctccgataccccatcatcacccctcggcctgcctgtggtggggacaaacctggcgtgagaactgtctacctctaccccaaccaaaccggcctccctgacccactgtcaaagcaccatgccttctgcttccgaggtgtgtcagtggcgccctctccaggagaagaagagggtagtacacccacatcaccctctgacatagaggactggatcgtcactcaggtggggcctggtgtggatgctgtccccttggagccaaagacaacagaagtgccatatttcaccactgagccaagaaaacagactgaatgggagccagcctacaccccagtgggcacatccccacagccagggatccctcccacatggcttcccaccctcccagcagcagaggaacacacagaaagcccctctgcctctgaagagccctctgcctcagcagtcccttccacctcagaggagccatacacatcttcatttgcagtgccgagcatgacagagctgccaggctctggggaggcgtcgggcgcacctgacctcagtggtgacttcacaggcagtggagatgcttcaggacgccttgactccagtgggcagccttcagggggcattgaaagtggccttccctcaggtgaccttgactccagtggcctcagccccacagtgagctcaggcctgcctgtagaaagtggttctgcctcaggagatggagaagtcccctggtcccatactcccacagttggcaggttgccctctggaggtgagagccccgaaggctctgcctctgcctctggaacaggagaccttagtgggctgccttcaggaggagaaattacagaaacttctacttctggggcagaagaaaccagtggacttccttctggaggagacggtctagaaacttctacctctggagtagatgatgtcagtggaattcctactggaagagaaggtctagagacttctgcctctggagtagaggacctcagtggacttccttctggagaagaaggttcagaaacatctacctctggaatagaggacatcagtgtacttccaactggaggagaaagtctagaaacctctgcttctggagtgggagacttgagtggacttccctcaggaggagaaagtctagaaacatctgcttcaggtgcagaggatgtcactcagcttcctactgaaagaggaggtctagagacttctgcctctggagtagaagacatcactgttcttcctactggaagagaaagtctagaaacttctgcctctggagtagaggatgtcagtggacttccttctggaagagaaggtctagagacttctgcctctggaatagaggacattagtgtgtttcctactgaagcagaaggtctggacacttctgcctctgggggatatgttagtgggattccttctggaggagatggtacagaaacctctgcttctggagtagaggatgtgagtggtcttccatctggaggagagggtctagaaacttctgcctctggagtggaagatcttggtccttctactagagatagtctagagacatctgcttcaggagtagatgttactgggtttccttctggaagaggggacccagagacctctgtttctggggtaggtgatgacttcagtggacttccttctggaaaagaaggcctggagacctcagcttctggagctgaggacctcagtggcttgccctctggaaaagaagacttggtagggtctgcttctggggccttggactttggcaaactacctcctggaactctaggaagtggtcaaactccagaagtaaatggctttccctctggatttagtggtgagtattctggagcagacattggaagtggcccatcctctggcctgcctgactttagtggacttccatctggctttccaactgtctcccttgtggacagtaccttagtggaagtgatcacagccaccacttccagtgaactggaaggaagggggaccattggcatcagtggttcaggagaagtatcagggctgcccctgggtgaattggacagtagtgcggacattagtggtctcccttcaggaactgaactcagtggccaagcatctggatctcctgatagcagtggagaaacatctggattttttgatgttagtggacagccatttgggtcttctggcgtcagcgaggaaacatctgggattcctgaaatcagtgggcagccatcagggactcctgacaccactgcgacatctggagtgactgagcttaatgaactgtcctctggacaaccagatgtcagtggagatgggtctggaattctctttggcagtggacagtcctctggtataacatctgtgagtggagaaacctctgggatttctgatctcagtgggcagccctcagggttcccagtgttcagtggaacagcaaccagaacccctgacctggcttctggcaccataagtggcagtggagagtcttctggcattacatttgtggacaccagttttgttgaagtgacccctaccacatttagggaagaagaagggttaggatctgtggaactcagtggctttccttctggggagacggaactgtctggcacatctgggacggtggacgtcagtgaacaatcttctggagcaattgattccagtggactcacatcccccactccagagttcagtggcctcccaagtggagtagctgaggtcagtggtgaattctctggagttgagactgggagcagcttgccctcaggagcatttgatggcagtggacttgtctcaggtttccccactgtgtctcttgtagacagaactttggtggaatctataactcaggctcctactgctcaagaagctggagaaggaccttcgggcattttggaattcagtggtgcccattctgggacaccagacatatctggggagctttctgggtctctggacctaagcacattgcagtctgggcagatggaaaccagcacggagacaccaagctctccatattttagtggagacttttccagcaccactgatgtaagtggagaatccatagctgccacaactggcagtggggaaagctctgggcttccggaagttactttaaacacctcagagttagtggagggtgtgactgaacccactgtttcccaggaacttggccatggtccttctatgacatacatctcccggctctctgaggccagtggggacgcctcagcatccggggaccttggtggcgctgtaacaaacttcccagggtctggagtagaagcttcagtcccagaagccagcagtgacctgtctgcttaccctgaggctggtgtgggagtgtctgctgcccctgaggccagcagtaaactgtctgagttcccagatctgcatggaatcacttctgccttccatgaaacagatctggaaatgacaaccccaagcacagaggtaaacagcaacccatggacctttcaggaaggcaccagggagggatcggccgctccggaagtgagtggagaatctagcactacctccgacatagacacaggcacttcaggggtgccttctgccacacccatggcttctggagacaggactgaaatcagcggagaatggtctgatcacacctcagaggtgaatgttgccatcagcagcaccatcacagagtccgagtgggcccagcctacccggtaccctacagagacacttcaagaaatcgaatccccaaatccctcatactcaggagaggagacccagacagcagaaacaaccatgtccctgacagatgcccccaccctctcttcttcagaagggtcaggggagacggagtcaaccgttgcagaccaggagcaatgtgaggaggggtggaccaagttccagggtcactgttaccgccactttcctgaccgagagacctgggtggatgcggagagacggtgtcgggagcagcagtcacatctgagcagcattgtcactcctgaggaacaggagttcgtcaacaaaaatgctcaagactaccagtggatcggtctgaatgacaggactatcgaaggggacttccgctggtctgacggacactctctgcaatttgagaagtggcgtccaaaccagcctgacaacttctttgccaccggagaggactgtgtggtgatgatctggcatgagagaggcgaatggaacgacgtcccctgcaattaccagctgcccttcacgtgtaaaaagggcaccgtggcctgtggagaccccccagtggtggagcatgctagaaccctcgggcagaagaaagatcgctacgagatcagctccctggtgcggtaccagtgcactgagggctttgtccagcgccacgtgcccaccatccggtgccagcccagcgggcactgggaagagcctcgaatcacctgcacagaccccaacacctacaagcacaggctacagaagcggacgatgagacccacacggaggagccgccccagcatggcccactgagaggagcttccataatgtgcccaggatgctgagcccagcggccagccaggctgaccgtgcatcccacccacatggtgtcttcttgtcgctttttgtcatataaggaatccattaaagaaggaaaaaaaacccacattgtgtgtgccccactccccgcatcacccaaactgcatctaatttatccccttctcccccccccccaaaaaaaaaaagaaaagaaatgcccaagcaagtcactgtaaccccgtggtacctcagctttgagacttttttgatttccccacccgtgccctttcccagggaccagtgcaggcacagggtgagaaggtaaggggttaaattaaataaagaagattcttttttgttgttgtttcctgactttgcccaagaacagtacaatggttggttacttcgcctccagggagagctaggggcccagaggaggagctgaaagaagacaggaccaggaagggaggagtagggcggtcacagagcttggaggactcagaagaggcaaagtacagcttagtgtgttggacaaaaggaattgaggtctgtgccatctgtgagggaaggacctggggcagggacaggctggctgaagagaagcagagccctctcctagggacctttcatctgggtcaaccaacctagcaggtgtcatttctcggctggactgggtaggggcacttccttcccaggggtctccgccgtccccttccctctccccaccccgggacggtgcaggcaccagtgttccgtgcacctatttatatttttgaaaactaaagattatgataattataataataaagacattggaagagatctaaaaaaaaaaaaaaaaaaaa

SEQ ID NO: 2 - protein, 2132 aa, Mus musculus
MTTLLLVFVTLRVIAAVISEEVPDHDNSLSVSIPQPSPLKVLLGSSLTIPCYFIDPMHPVTTAPSTAPLTPRIKWSRVSKEKEVVLLVATEGQVRVNSIYQDKVSLPNYPAIPSDATLEIQNLRSNDSGIYRCEVMHGIEDSEATLEVIVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQGGMDMCSAGWLADRSVRYPISKARPNCGGNLLGVRTVYLHANQTGYPDPSSRYDAICYTGEDFVDIPENFFGVGGEDDITIQTVTWPDLELPLPRNVTEGEALGSVILTAKPIFDLSPTISEPGEALTLAPEVGSTAFPEAEERTGEATRPWGFPAEVTRGPDSATAFASEDLVVRVTISPGAAEVPGQPHLPGGVVFHYRPGSTRYSLTFEEAQQACMHTGAIIASPEQLQAAYEAGYEQCDAGWLQDQTVRYPIVSPRTPCVGDKDSSPGVRTYRVRPSSETYDVYCYVDKLEGEVFFATRLEQFTFQEARAFCAAQNATLASTGQLYAAWSQGLDKCYAGWLADGTLRYPIITPRPACGGDKPGVRTVYLYPNQTGLPDPLSKHHAFCFRGVSVAPSPGEEEGSTPTSPSDIEDWIVTQVGPGVDAVPLEPKTTEVPYFTTEPRKQTEWEPAYTPVGTSPQPGIPPTWLPTLPAAEEHTESPSASEEPSASAVPSTSEEPYTSSFAVPSMTELPGSGEASGAPDLSGDFTGSGDASGRLDSSGQPSGGIESGLPSGDLDSSGLSPTVSSGLPVESGSASGDGEVPWSHTPTVGRLPSGGESPEGSASASGTGDLSGLPSGGEITETSTSGAEETSGLPSGGDGLETSTSGVDDVSGIPTGREGLETSASGVEDLSGLPSGEEGSETSTSGIEDISVLPTGGESLETSASGVGDLSGLPSGGESLETSASGAEDVTQLPTERGGLETSASGVEDITVLPTGRESLETSASGVEDVSGLPSGREGLETSASGIEDISVFPTEAEGLDTSASGGYVSGIPSGGDGTETSASGVEDVSGLPSGGEGLETSASGVEDLGPSTRDSLETSASGVDVTGFPSGRGDPETSVSGVGDDFSGLPSGKEGLETSASGAEDLSGLPSGKEDLVGSASGALDFGKLPPGTLGSGQTPEVNGFPSGFSGEYSGADIGSGPSSGLPDFSGLPSGFPTVSLVDSTLVEVITATTSSELEGRGTIGISGSGEVSGLPLGELDSSADISGLPSGTELSGQASGSPDSSGETSGFFDVSGQPFGSSGVSEETSGIPEISGQPSGTPDTTATSGVTELNELSSGQPDVSGDGSGILFGSGQSSGITSVSGETSGISDLSGQPSGFPVFSGTATRTPDLASGTISGSGESSGITFVDTSFVEVTPTTFREEEGLGSVELSGFPSGETELSGTSGTVDVSEQSSGAIDSSGLTSPTPEFSGLPSGVAEVSGEFSGVETGSSLPSGAFDGSGLVSGFPTVSLVDRTLVESITQAPTAQEAGEGPSGILEFSGAHSGTPDISGELSGSLDLSTLQSGQMETSTETPSSPYFSGDFSSTTDVSGESIAATTGSGESSGLPEVTLNTSELVEGVTEPTVSQELGHGPSMTYISRLSEASGDASASGDLGGAVTNFPGSGVEASVPEASSDLSAYPEAGVGVSAAPEASSKLSEFPDLHGITSAFHETDLEMTTPSTEVNSNPWTFQEGTREGSAAPEVSGESSTTSDIDTGTSGVPSATPMASGDRTEISGEWSDHTSEVNVAISSTITESEWAQPTRYPTETLQEIESPNPSYSGEETQTAETTMSLTDAPTLSSSEGSGETESTVADQEQCEEGWTKFQGHCYRHFPDRETWVDAERRCREQQSHLSSIVTPEEQEFVNKNAQDYQWIGLNDRTIEGDFRWSDGHSLQFEKWRPNQPDNFFATGEDCVVMIWHERGEWNDVPCNYQLPFTCKKGTVACGDPPVVEHARTLGQKKDRYEISSLVRYQCTEGFVQRHVPTIRCQPSGHWEEPRITCTDPNTYKHRLQKRTMRPTRRSRPSMAH

SEQ ID NO: 3 - DNA, 2291 bp, Mus musculus
  CDS (257)..(1363)
aagaggtaatccagaactcaaggagcgcgggcgcagcagctgtgagcattaggtgctgtggccaagcgctgggacaaggatctgccgctaccagcgcatcctctctcctctccagctctgtcccgcactcgctgcacccttccccaggccaccgcgcttcctatgtgatcttccggggcaacgcggagccctttctcacagctcagcagtgaatttccaccccgaaaactgcagtaagccgcctttcaaggacaagatgttgataaatatgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatatcggggcggtcatagcaagcccagagcagctgtttgccgcctatgaggatggatttgagcagtgtgatgcaggatggctgtctgatcaaactgtcagatatcccatacgggctccccgagagggctgttacggagacatgatggggaaggaaggggttcggacctatggattccgctctccccaggaaacctatgatgtgtattgttatgtggatcatctggatggcgatgtgttccacatcactgctcccagtaagttcaccttcgaggaggccgaagcagagtgtacaagcagggatgcgaggctggcgactgttggagaacttcaggcagcttggagaaatggctttggccaatgcgattacggctggctgtcggatgccagcgtgcggcaccctgtgactgtggccagggcccagtgtggaggaggtctacttggggtgagaaccctgtatcgttttgagaaccagacatgcttccctctccctgatagcagatttgatgcctactgctttaaacgtaagtgtttgatatctcttttaaaaatacagattaaaaagctgcagcaaatatatacccactgaggttccagtggatgtcgggatggattctgtattctgtactaagtgcatggaggacctacagtgcagtgacacaaacagacttaataccaccattatggttaacacagtagggagtttggcaagtgggattttttttgggaggtggggagataacaacgtcagagtggggtcttaactgcaccccagaattccaaaaatcacagaacacaccctgaattgaaatttcttattaattgaagcctttcaatgcaatttcactcttaatataacaatgttaagacaacagcagctgagttaaacctttgaatcctgttttattttttgatatgattcttattataaaaaggagacaatttaagcttaagtcattgcaggtgagtatttgtgctgaaattaataatgattgtgttcaattcctacctaaaaatgcatcatactatcaaataatacgattcacatatctcattttgtttcaagtaatagaataggcgccttttggcgattaatcttgctttgaattaattaaagtacaatacagcctccttgcatgaattggctatgctcagttagatttgccttgtaaaattaattttaggtagcattcattacatatatatatatgtatatatatatattagacattcttaaattattttaaaatattagtcacatacagttttatagttgtagagtgcttaactaaactaataactagtcaatattttattgtaatatgtactttaggatcggtaagatgttagctaacaaatgcctccatactttagcaacagaggccaagtctgacatcgagtatcagaacaatgaaaagccaatgtaacacatgaagtcttttaaaccagctttctaattccttttgttagatcaatgggtaattaaaatagcttgaaaatgtaaaaaaaaaaaaaaa

SEQ ID NO: 4 - protein, 368 aa, Mus musculus
MLINMKGILWMCSTLLLTHALHQAKMETSPPVKGSLSGKVVLPCHLSTLPTLPPNYNTSEFLRIKWSKMEVDKNGKDIKETTVLVAQNGNIKIGQDYKGRVSVPTHPDDVGDASLTMVKLRASDAGVYRCDVMYGIEDTQDTMSLAVDGVVFHYRAATSRYTLNFAAAQQACLDIGAVIASPEQLFAAYEDGFEQCDAGWLSDQTVRYPIRAPREGCYGDMMGKEGVRTYGFRSPQETYDVYCYVDHLDGDVFHITAPSKFTFEEAEAECTSRDARLATVGELQAAWRNGFGQCDYGWLSDASVRHPVTVARAQCGGGLLGVRTLYRFENQTCFPLPDSRFDAYCFKRKCLISLLKIQIKKLQQIYTH

SEQ ID NO: 5 - DNA, 2583 bp, Mus musculus
  CDS (162)..(2339)
cggcagtggctggcaagaaacaattctgcaaaaataatcatacccagcctggcaattgtctgctcctcggtccattgctccgccgccgtccacagtcgcttgcaaggggaaggcactgaatttaccgcggccagaacatccctcccagccggcagtttacaatgctgcgaactaaggatctcatctggactttgtttttcctgggaactgcagtttccctgcaggtagatattgttcccagccaaggagaaatcagcgttggagagtccaaattcttcctgtgtcaagtggcaggagatgccaaagataaggacatctcctggttctcccccaatggggagaagctgagcccaaaccagcagcggatctcagtggtgtggaatgatgacgactcctctaccctcaccatctacaacgccaacatcgacgatgccggcatatacaagtgtgtggtcaccgctgaggacggcacccagtctgaggccactgtcaacgtgaagatcttccagaagctcatgttcaagaatgcaccaaccccacaggagtttaaggaaggggaggatgctgtgattgtctgtgatgtggtcagctccctgcctccaaccatcatctggaaacacaaaggccgagatgtcattctgaaaaaagacgtccggttcatagtcctgtccaacaactacctgcagatcaggggcatcaagaaaacagatgagggtacttaccgctgtgagggcaggatcctggcccgcggggaaatcaacttcaaggacattcaggtcattgtgaatgtaccacccactgtccaggccagacagagcatcgtgaatgccactgccaacctgggccagtctgtcaccctggtgtgtgatgctgatggatttccagagcccaccatgagctggacaaaggatggggaacccatagagaacgaggaggaagatgagagaagcagatcttcagtgagtgacagctccgaggtgaccatcaggaatgtggataaaaacgacgaggccgaatacgtctgcatcgcagagaacaaggctggcgagcaggatgcctccatccacctcaaggtctttgcaaagcccaaaatcacctatgtagagaatcagacggccatggaactagaggagcaagtcactctgacctgtgaagcctccggagaccccatcccgtccatcacgtggagaacatccacccggaacatcagcagtgaagaacaggatctggatgggcacatggtggttcgcagccatgctcgtgtgtcctccttgaccctgaagagcatccagtacagagatgctggagaatacatgtgcactgccagcaacaccatcggccaggactcccagtccatcgacctcgaatttcaatatgctcccaagctccagggccctgtggctgtctacacctgggaagggaaccaagtgaacatcacctgtgaggtctttgcctatcccagtgccacaatctcctggttccgagatggtcagttgctgccaagctccaactacagcaatatcaagatctacaacaccccatctgctagctatctggaggtgaccccagattcagaaaatgactttggaaactacaactgtacagcagtgaaccgtattggacaggagtccttggaattcatccttgttcaagcagacacaccgtcttctccatccattgaccgggtggaaccatactccagcacagcgcaggtgcagtttgatgagccagaggccacagggggcgtgcccattctcaagtacaaggctgagtggaagtcgctgggcgaagaatcctggcatttcacgtggtatgatgccaaagaagccaacatggagggcattgtcaccatcatgggcctgaaacctgagacgacgtactcggatcgactggcggccctcaacggcaaggggctgggcgagatcatgcagccatctgagtccaagacacagccagttccggaactcagtgcacccaagctggaagggcagatgggagaggacgggaactccatcaaggtgaacctgatcaagcaggatgacggaggctcccccatcagacactatctggtcaagtacagagcgctcgcctctgagtggaaaccggaaatcaggctcccatccggcagtcaccacgtcatgctcaagtccctggactggaacgcagagtatgaagtctatgtggtagctgaaaaccagcaaggaaaatccaaggcagctcactttgtgttcaggacctcagcccagcccacggccatcccagccaccctgggcggaagctccacctcctacaccttggtctcattgcttttctctgcggtgactcttcttctgctctgatagaaacttgaacatgcatgtgtgcgagctctcatgtgcgcatgcgcacacgcacacacacgcacacgcacacgcacacgcacgcacacgcacacacacacacacacagacacacacacgcacacacacacgcacatgcacacgcgcacacacgcacacgcacacgcacacgcacgcacacacacacacacacacaccattgtatttgattaaaaagcccagttcctataaaaaaaaaaaaaaa

SEQ ID NO: 6 - protein, 725 aa, Mus musculus
MLRTKDLIWTLFFLGTAVSLQVDIVPSQGEISVGESKFFLCQVAGDAKDKDISWFSPNGEKLSPNQQRISVVWNDDDSSTLTIYNANIDDAGIYKCVVTAEDGTQSEATVNVKIFQKLMFKNAPTPQEFKEGEDAVIVCDVVSSLPPTIIWKHKGRDVILKKDVRFIVLSNNYLQIRGIKKTDEGTYRCEGRILARGEINFKDIQVIVNVPPTVQARQSIVNATANLGQSVTLVCDADGFPEPTMSWTKDGEPIENEEEDERSRSSVSDSSEVTIRNVDKNDEAEYVCIAENKAGEQDASIHLKVFAKPKITYVENQTAMELEEQVTLTCEASGDPIPSITWRTSTRNISSEEQDLDGHMVVRSHARVSSLTLKSIQYRDAGEYMCTASNTIGQDSQSIDLEFQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDRVEPYSSTAQVQFDEPEATGGVPILKYKAEWKSLGEESWHFTWYDAKEANMEGIVTIMGLKPETTYSDRLAALNGKGLGEIMQPSESKTQPVPELSAPKLEGQMGEDGNSIKVNLIKQDDGGSPIRHYLVKYRALASEWKPEIRLPSGSHHVMLKSLDWNAEYEVYVVAENQQGKSKAAHFVFRTSAQPTAIPATLGGSSTSYTLVSLLFSAVTLLLL

SEQ ID NO: 7 - DNA, 3153 bp, Mus musculus
  CDS (151)..(2802)
  misc_feature (3043)..(3043): n is a, c, g, or t
gaggctcccggcgagctggcgcccctgtctgggtcccgcgcgcccggccctgctcgcgcccgcgcatcgcgccgcagtctcggtctgcggctgcgggacgtgacggcgtgcgcggaggggacctcgcaagttcttccatcagtgtgcagaatgataccactgcttctgtccctgctggccgctctggtcctgacccaagcccctgccgccctcgctgatgacctgaaagaagacagctcggaggatcgagccttccgcgtgcgcatcggtgccgcgcagctgcggggcgtgctgggcggtgccctggccatcccatgccacgtccaccacctgcggccgccgcgcagccgccgggccgcgccgggttttccccgggtcaagtggaccttcctgtccggggaccgggaggtagaggttctggtggctcgcgggctgcgcgtcaaggtaaacgaagcctaccggttccgcgtggcgctgcctgcctaccccgcatcgctcacggatgtgtctctagtattgagcgaactgcggcccaatgattccggggtctatcgctgcgaggtccagcacggtatcgacgacagcagtgatgctgtggaggtcaaggtcaaaggggtcgtcttcctctacagagagggctctgcgcgctatgctttctccttcgctggagcccaggaagcctgcgctcgcataggagcccgaatcgccaccccggagcagctctatgctgcctacctcggcggctatgagcagtgtgatgcaggctggctgtccgaccaaactgtgaggtaccccatccagaacccacgagaggcctgctctggagacatggatggctatcctggcgtgcggaactacggagtggtgggtcctgatgatctctatgatgtctactgttatgccgaagacctaaatggagaactgttcctaggcgcccctcccagcaagctgacatgggaggaggctcgggactactgtctggaacgtggtgcacagatcgctagcacaggccagctgtacgcagcctggaatggtggcctggacagatgtagccctggctggctggctgatggcagcgtgcgctatcccatcatcacacccagccaacgctgtgggggcggcctgccaggagtcaagaccctcttcctctttcccaaccagactggcttccccagcaagcagaaccgcttcaatgtctactgcttccgagactctgcccatccctctgcttcctctgaggcctctagcccagcctcagatggacttgaggccattgtcacagtgacagaaaagctggaggaactgcagctgcctcaggaagcgatggagagcgagtctcgtggggccatctactccatccccatctcagaagatgggggaggaggaagctccaccccagaagacccagcagaggcccccaggactccgctagaatcggaaacccaatccattgcaccacctaccgagtcctcagaagaggaaggcgtagccctggaggaagaagaaagattcaaagacttggaggctctggaggaagagaaggagcaggaggacctgtgggtgtggcccagagagctcagcagccctctccctactggctcagaaacagagcattcactctcccaggtgtccccaccagcccaggcagttctacagctggatgcgtcaccttctcctgggcctccaaggttccgtggaccgcctgcagagactttgctccccccgagggagtggagcgccacatctactcctggtggggcaagagaagtagggggggaaactgggagccctgagctctctggggttcctcgagagagcgaggaggcagggagctccagcttggaggatggcccttccctacttccagctacatgggcccctgtgggtcccagggagctggagaccccctcagaagagaagtctggaagaactgtcctggcaggcacctcagtgcaggcccagccagtgctgcccaccgacagtgccagccacggtggagtggctgtggctccctcatcaggtgactgtatccccagcccctgccacaatggtgggacatgcttggaggagaaggagggtttccgctgcctatgtttgccaggctatgggggggacctgtgcgatgttggccttcatttctgcagccctggctgggaggccttccagggagcctgctacaagcacttttccacacgaaggagttgggaggaggcagaaagtcagtgccgagcgctaggtgctcatctgaccagcatctgcacccctgaggagcaagactttgtcaatgatcgataccgggagtaccagtggattgggctcaatgacaggaccatcgagggtgacttcttgtggtcagatggtgcccctctgctctatgaaaactggaaccctgggcagcctgacagctacttcctgtctggggagaactgtgtggtcatggtgtggcatgaccagggacagtggagtgatgtgccctgcaactaccatctatcctacacctgcaagatggggcttgtgtcctgtgggcctccaccacagctacccctggctcaaatatttggtcgccctcggctgcgctacgcggtggatactgtgcttcgatatcgatgccgagacgggctggctcagcgcaacctgccgttgatccgctgccaggagaatgggctttgggaggcccctcagatttcctgtgtaccccggaggcctggccgtgctctgcgctccatggacgccccagaaggaccacggggacagctctcgaggcacaggaaggcaccgttgacaccgccctccagtctctagggagcctggaagactgctgcccccagcaggaccctctcacatcaactgccagtgctcttccccatgatagggggtgacgtgagaggggtgggactgaaattcagaggacagcgctcgaaggggtttctgggaaacacttgggtggctccgccccctcacacaagggcctcaggttttacccggtaagtccctaagtgcctcaactgccctctcatgtcagctgcctccttgtccctcgatntcgtnaggggacactgtgctattcgatcttgattgtcgaagagtttttaggatggagtaccagcaaaaccaggtggaaataaagttgtctgaacccaaagaaaaaaaaaa

SEQ ID NO: 8 - protein, 883 aa, Mus musculus
MIPLLLSLLAALVLTQAPAALADDLKEDSSEDRAFRVRIGAAQLRGVLGGALAIPCHVHHLRPPRSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACSGDMDGYPGVRNYGVVGPDDLYDVYCYAEDLNGELFLGAPPSKLTWEEARDYCLERGAQIASTGQLYAAWNGGLDRCSPGWLADGSVRYPIITPSQRCGGGLPGVKTLFLFPNQTGFPSKQNRFNVYCFRDSAHPSASSEASSPASDGLEAIVTVTEKLEELQLPQEAMESESRGAIYSIPISEDGGGGSSTPEDPAEAPRTPLESETQSIAPPTESSEEEGVALEEEERFKDLEALEEEKEQEDLWVWPRELSSPLPTGSETEHSLSQVSPPAQAVLQLDASPSPGPPRFRGPPAETLLPPREWSATSTPGGAREVGGETGSPELSGVPRESEEAGSSSLEDGPSLLPATWAPVGPRELETPSEEKSGRTVLAGTSVQAQPVLPTDSASHGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLRYAVDTVLRYRCRDGLAQRNLPLIRCQENGLWEAPQISCVPRRPGRALRSMDAPEGPRGQLSRHRKAPLTPPSSL

SEQ ID NO: 9 - DNA, 3977 bp, Mus musculus
  CDS (232)..(2784)
gaattcgcggccgcgtcgaccggatggcgcagttgaactgcgctgctgagctcgcggccgcctgcgcgcgctggggggactcgtctgggctactcccccacctcgcgttggacgaccggtcctggacacaccgcttgcgaggcaagttgaacattgcagagaaggatcttaaggctacacccgacttgccacacttgccatccagctgaagaaccaaaggctgttggagagatggcagtgacatcccaccacatggtccctgtgtttgtcctgatgagcgcctgcctggccaccgcaggtccagagcccagcacccggtgtgaactgtcaccgatcagtgcctctcatccagtccaggccctgatggagagcttcaccgttctgtctggctgtgccagcagaggcaccactgggctgccaagggaggttcacatcctaaacctccgcagtacagaccaaggactaggccagccgcagagagaggttaccctgcatctgaaccccattgcctccgtgcacactcaccacaagcctgttgtgttcctgctcaactccccacagcccctggtgtggcatgtgaagacagagagactggctgctggtgtccccagactcttcctggtttcggagggttctgtggtccagttttcatcaggaaacttctccttgacagcagaaacagaggaaaggagtttccctcaagaaaatgagcatctgctacactgggcccaaaaggaatatggagcagtgacttcattcaccgaacttaagatagcaagaaatatctatattaaagtgggagaagatcaagtgttccctcccacgtgtaacatagggaaaaattttctctcgctcaattaccttgcggagtaccttcaacccaaagccgccgaaggttgtgtcctggccagtcagccccacgagaaggaagtgcatatcattgagttaatctcccccaactccaatccttacagcaccttccaggtggatataataattgacatacgacctgctcgagaggatcctgaggtggtcaaaaacctcgtcctgatcttgaagtgcaaaaaatctgtcaactgggtgatcaagtcttttgacgtcaagggaaacttgaaagttattgctcctgacagtattggctttggaaaagagagtgaacgatccatgacagtgaccaaattggtaagaaatgactacccttccacccaagagaatctgatgaagtgggcattggacaatggctacagcccagtgacgtcatacaccatagctcctgtggccaatagatttcatcttcggcttgagaacaacgaggagatgagagatgaggaagtccacaccattcctcctgagcttcggatcctgctgggccctgaccacctgcctgccctggacagcccacccttccaaggggaaatcccaaatggaggtttcccctttccattcccggatatccccaggagaggctggaaggagggagaagataggatcccccggccaaaggaacccatcattcccagagttcaattgcttccagaccacagagagccagaagaagtgcaagggggcgtgaatatcgccctgtcagtcaaatgtgacaatgaaaagatggtggtagctgtagacaaagattctttccagaccaatggctactcggggatggagctcaccctgttggatccttcctgcaaagccaagatgaatggtacccactttgttctggagtctccgctgaatggctgtggtactagacatcggaggtcagccccagatggtgtggtttactataactctattgtggtgcaggctccatcccctggggatagcagtggctggccagacggctacgaagatttggagtcgggtgataatggatttcctggagacacagatgaaggagaaactgcccccctgagccgtgctggagtggtagtgtttaactgcagcttgcggcagctgaggagtcccagtggcttccaggaccagctcgatggaaatgctaccttcaatatggagctgtataacacagacctctttctggtgccctccccgggggtcttctctgtggcagagaatgagcatgtatatgttgaggtgtctgtcactaaggctgaccaagatctgggatttgccatccaaacctgctttatctctccatactcaaacccagacagaatgtctgattacaccatcatcgagaacatctgtccgaaggatgactctgtgaagttctacagctccaagagagtgcacttccccatcccacatgctgaagtggacaagaagcggttcagctttgtgttcaagtccgtgttcaacacctccctgctcttcctgcactgcgagctgacgctgtgctctaggaacaagggctcccagaagctgccaaagtgtgtgactcctgatgacgcctgcacctctctagatgccaccatgatctggaccatgatgcagaataagaagacattcaccaagcccctggccgtggtcctccaagtagattataaagaaaatgttccaaacatgaaggagtccagtccggttcctcctcctccacagattttccacggcctggacacgctcaccgtgatgggcattgcgtttgcagcatttgtgatcggagcactcctgacgggggccttgtggtatatctactcccacacaggggagacggcacgaaggcagcaagtccctacctcgccaccagcctcggagaacagcagcgcagcccacagcatagggagcactcagagcactccctgctctagcagcagcacggcctaggtggacagatggacgccgctgcctaggtggacagacggatgcctgccaccgccggggcagggccagatgccgatgctgggtgtccagactcagaaggcttgatctgggttccctgtaaagagagagtgaatttcagtatacagacagcctgctctacccaccccttccttaccacggcccacgtaaatgtgaccctggacatctgtcacacgaaagctaagctggtggccttccctgccagcccctcgcaggatgggggtttcaatgtgaaacatatgccagtttttgttctttttttttttttattttttatgctgctttgtccaggtgtccaaacatccatcatttggggtcgtatgttttaaagagtaaaggagacggtgaagggatgtcagctagagcgtagagccagggagagacagccagggattctcgcctagctgaaccaagatgtaaaataggagaaatgctccctccatagccaatctgcatctcgctctggatgcccactcctgacctgccacgccctacatggtcccctgtgtagcttgctagccttgggggcctgtcacttgccaaagtaacattcctctcttgggccaaaatgaatgctgactcctgatactccgcacaagcacaccaactccaaaagtgccttaaagccaggtttgtgtctcctttcaggagagagcgtgtaacgtcagatttgtgcctgtctcgaacattctgtcctaatcatacttaaacatcaggcaggtggggttcagtggtaagcgaagggattattagcaaggtaatgcctgtgtatggtcatataggcacaaatgcagaacacccagcacattggagagacgaccacttcctcctttaaaatgtcattgcttccagagtcggaacctgagttatatgtggtagattatattgactccaaccatcagcctttctgatgagcctttagagaagcctagagagcccgggcatctgatgatgtcacagtgctgcctcagatgatacagagacagggagtttcaggtgaacttcttcagcagaataaggtgatgccactctgacctttgctgagctcagtgactcttcagtgttaaaactgaacccagaggggattctccatgccagatccgctgcaaattaaataataccaattttctagagtccgtcgacgcggccgcgaattc

SEQ ID NO: 10 - protein, 850 aa, Mus musculus
MAVTSHHMVPVFVLMSACLATAGPEPSTRCELSPISASHPVQALMESFTVLSGCASRGTTGLPREVHILNLRSTDQGLGQPQREVTLHLNPIASVHTHHKPVVFLLNSPQPLVWHVKTERLAAGVPRLFLVSEGSVVQFSSGNFSLTAETEERSFPQENEHLLHWAQKEYGAVTSFTELKIARNIYIKVGEDQVFPPTCNIGKNFLSLNYLAEYLQPKAAEGCVLASQPHEKEVHIIELISPNSNPYSTFQVDIIIDIRPAREDPEVVKNLVLILKCKKSVNWVIKSFDVKGNLKVIAPDSIGFGKESERSMTVTKLVRNDYPSTQENLMKWALDNGYSPVTSYTIAPVANRFHLRLENNEEMRDEEVHTIPPELRILLGPDHLPALDSPPFQGEIPNGGFPFPFPDIPRRGWKEGEDRIPRPKEPIIPRVQLLPDHREPEEVQGGVNIALSVKCDNEKMVVAVDKDSFQTNGYSGMELTLLDPSCKAKMNGTHFVLESPLNGCGTRHRRSAPDGVVYYNSIVVQAPSPGDSSGWPDGYEDLESGDNGFPGDTDEGETAPLSRAGVVVFNCSLRQLRSPSGFQDQLDGNATFNMELYNTDLFLVPSPGVFSVAENEHVYVEVSVTKADQDLGFAIQTCFISPYSNPDRMSDYTIIENICPKDDSVKFYSSKRVHFPIPHAEVDKKRFSFVFKSVFNTSLLFLHCELTLCSRNKGSQKLPKCVTPDDACTSLDATMIWTMMQNKKTFTKPLAVVLQVDYKENVPNMKESSPVPPPPQIFHGLDTLTVMGIAFAAFVIGALLTGALWYIYSHTGETARRQQVPTSPPASENSSAAHSIGSTQSTPCSSSSTA

SEQ ID NO: 11 - DNA, 1767 bp, Mus musculus
  CDS (207)..(1271)
gactaatgttggggtaaacacagaaagcccattataacatgataagttcattaaaagaggtcagaatatcgtgcttcagatcacagaagcggtaacgagcagcaccctgctcagaggagaagtgaggggagacaggggcaggagacccagaatctcacgtaacctttaaacttcttcagaaggtcgtgaaaatacatgagataatcatgaaggcaactctcatcttcttccttctggcacaagtctcttgggctggaccatttgaacagagaggcttatttgacttcatgctagaagatgaggcttctggcataatcccttatgaccctgacaatcccctgatatctatgtgcccctaccgatgccagtgtcatcttcgagtggtgcagtgttctgatctgggtttggacaaagtgccctgggattttccacccgacacaaccttgctagacctgcaaaacaacaaaattacagagatcaaagaaggggccttcaagaacctgaaggacttgcataccttgatccttgtcaacaacaagatcagcaaaatcagtccagaggcattcaaacctctcgtgaagttggaaaggctttacctgtctaagaaccaactaaaggaactgcctgaaaaaatgcccagaactctccaggaacttcgtgtccatgagaatgagatcaccaagctgcggaaatccgacttcaatggactgaacaatgtgcttgtcatagaactgggcggcaacccactgaaaaactctgggattgaaaacggagccttccagggactgaagagtctctcatacattcgcatctcagacaccaacataactgcgatccctcaaggtctgcctacttctctcactgaagtgcatctagatggcaacaagatcaccaaggttgatgcacccagcctgaaaggactgattaatttgtctaaactgggattgagcttcaacagcatcaccgttatggagaatggcagtctggccaatgttcctcatctgagggaactccacttggacaacaacaaactcctcagggtgcctgctgggctggcacagcataagtatatccaggtcgtctaccttcacaacaacaacatctccgcagttgggcaaaatgacttctgccgagctggacacccctctcgaaaggcttcctactcggctgtgagtctttacggcaaccctgtccggtattgggaaatctttccaaacaccttcagatgtgtctatgtgcgttctgccattcaacttggaaactacaagtaaccctcagacggcctaattcttataatctggaaaaacaccccaatatgtcaatatcattgctaaaaagaaaaaatactaaaaaagaaagaaagaatgctagattctgaaaattcaagcacactgtgcatgccttttccacatgacttattatgcaagctgaatatacagattgaataattgcctacaataaaaatattttacttgtagtattcagaatcacttttaaagttgtctgtagttgtgaactgagttatcaaagtctgatgtaatcataaatgtcaaccacttagtaaagcgttaaaaggaacagaatacaaagtctctgtaattcatagagctaaattaactcttttgacataaagtcaaatgccgccgaactctagcaatgtattaatctcctttattattggtgaagccttatttgagtatgtaagttcaatatgggctatgtaaaattttcactgtcatattgaaaaaaataaaattttaaaaatgc

SEQ ID NO: 12 - protein, 354 aa, Mus musculus
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 13 - DNA, 1474 bp, Mus musculus
  CDS (152)..(556)
gcctctgtctccctctctccacgaactgcccaggagcgagcagctgctcccggttggccctgacggacagacaaaccgacagcctgacaacctagtccaccaactaagcagcctgcacctggctgcttgtccctccccaggaacattgaccatgtgtcccctgtggctactcaccttgctgctggccctgagccaggccttgccctttgagcagaagggtttctgggacttcaccttggatgatgggctgctcatgatgaatgatgaggaggcttcaggttcagacaccacttcaggtgtccccgacctggactctgtcacacctaccttcagtgccatgtgtcctttcggttgccactgccacctgcgggttgttcagtgctctgacttgggtctgaagactgtgcccaaggagatctcacctgacaccacactgctagacctgcagaacaatgacatttctgagcttcgcaaggatgacttcaaaggcctccagcacctctacgtaagcctcctcctgcctcctgccttttctgaggagccaggaggagtttgatgttcctatgacttggaagaagggtactcacaaatgaggttgggaagaggggtgggaaccgatggagtcctgagataagtctggagctggctgtaatgagaacgcccaaaaccatgcattatatgtatgggtacaagacgaaatctgtcaggtcccatttttttttgacagaaatctttacgttttgttgactcatctgaacaacacagaaagtgtattccatcatagctcttagagaaactgtctagcaaagtatgctaggcagtgatggcattaccagggcagcttggagctaacctcagctccttgccctacactctactcaccaacacatactgatctccatgagaccattaaaggttcagatgtttcccaagtgccatcagtagcaagttcaggaggcagaggagaggggaggtggagtggggggctggggtgctcaccctggttgcctgccccgttttaggccctggtcttggtaaacaataagatctccaagatccatgagaaggcctttagccctctgcggaagctgcaaaaactctacatctccaagaaccacctggtggagattcctcccaacctgcccagctccctggtagaactacgaatccatgacaaccgtatccgcaaagtgcccaagggcgtgttcagcgggctccggaacatgaactgcattggtgagactagtctgtaccttagcatcaggcataaaggacacaggtagaggatatctctttcagccagggcagctgggtccatgtagtcatggaaaagccctgtgtgcctcagaatgttccagatgtttgttttgggacatgaagaaaaagactgtggtggctataaaggctcggttgaagaaagagtctcaaatatagtctaaaaaagaccagtatgtttctgaaaaaaaaaaaaaaa

SEQ ID NO: 14 - protein, 134 aa, Mus musculus
MCPLWLLTLLLALSQALPFEQKGFWDFTLDDGLLMMNDEEASGSDTTSGVPDLDSVTPTFSAMCPFGCHCHLRVVQCSDLGLKTVPKEISPDTTLLDLQNNDISELRKDDFKGLQHLYVSLLLPPAFSEEPGGV

SEQ ID NO: 15 - DNA, 2860 bp, Mus musculus
  CDS (78)..(1208)
gctgccacattctccaacccaaggagaccagacagaaggacgtggtcactctgaacagttcaacccaagagacaaaaatgcagtgggcctccgtcctgctgctggctgggctctgctcactttcccagggccagtatgatgaagactctcactggtggatccaatacctccgaaaccagcagtccacctactacgacccctatgacccttacccctatgagccctctgagccttacccctatggagtggaagaaggcccagcctatgcctatggtgcaccacctccaccagagccccgtgattgtccccaagaatgcgactgtccccccaacttccccacagccatgtactgtgacaaccgcaacctcaagtacctgccttttgtgccctcccgcatgaagtacgtctacttccagaacaaccagatctctgccatccaggaaggtgtctttgacaatgccactgggctcctctgggtcgctctacatggcaaccagattaccagtgacaaggtaggcaggaaagtcttctccaagctgaggcacttggagaggctgtacttggaccacaacaacctgacccggatgcccggcccgctgccccgatccctgagagagctccacctggaccacaaccagatctcacgggtccccaacaatgctttggagggcctggagaacctcacggccttatatctccaccacaatgagatccaggaagtggggagttccatgagaggcctccggtccctgatcctactagacctgagttataaccaccttcggagggtacccgacggtctgccctcagccctggagcagctgtacctagaacacaacaatgtctacaccgtccctgacagctacttccgggggtcacccaagctgctgtacgtccggttgtctcacaacagtctcactaacaacggccttgctaccaacaccttcaactccagcagccttcttgagctagacttgtcttacaatcagctgcagaagatccctcctgtcaacaccaacctggagaatctttacctccagggcaacaggatcaatgagttctccatcagcagcttctgcacggtggtggacgtcatgaacttctccaagctgcaggtgctacgcctggatgggaacgagatcaagcgcagcgcaatgccggtggacgccccactctgcctgcgcctcgccaacctcatcgagatctgaggggcctcgccttgggctcctggctccgggctccgagtaccgtgcaccgtgcaccgggctcggggtacatgccctgccaccccgctgcatgtttggcttttgctggatggtctgggacacgcatgtgacagaagtccacaggatcttattcaatctccttccaacaggcagagttaggtgggatcaggggccaggccagtttctgcagggggatgaatttggtggtaaaagaaatatagctatagagtctagccccaaaatcttcttgcttggacagtagcatataaccagttccaatttgaccttttttgagctgtgctcaacagcatggccagctgcttctgcagttgctctgggctcctgctctttgctcctcaaaacatcacaacacacctcttgcccagtcacctcctcccagccccagctcaccctctctgccctctttcactggggcctcttctaatgtcttaggcagtcaggagacacccacaccctaagggcacactcttatctgaggctaaacggatggctaaaatcacacacttacccccacctcacctgtttaaagtcaccatattgaacaccataatgatgagctgttaacataggggataaccgactctataatggaggatctatcagaggagggaagtgtccagtggtcattatcagtatccatagtaagatggggaagagacacacctcaggatagcagaactgaaggggcagccccctttccagtttcatctgacacaacatgatctgcaccccttcttgggcttttagtatcgaaggggcaagcgtggttttcaaaacatgagaaagagcctctgctcatcctttggtctagtatgaagggttgttacgcaaatggcagaggcagactccacccgccgaggacctacccactaacccagactagggggtgatctcaatagctcgtttgtccataatgctagagcatcaggaaccagccctgtggagatgctcactggggtgcaagaagccctgccaggcccaagtcctgccgacactagacctggcttcctctgcattcacactatgcagtccgccttatgcccagtggtgcctgtgatttgtagtttgtctaagaacagccccatcttctcctttttaaaggtgatattgcctaggtacaaccaacagcttcttctagaccccactgggatagagccaccagggtagcgagtggcaagaagcagttgtaagatgctagcctgaggtgggtccctgccttagacccctgactctgtgtgcacttgggagcaagcttctctggaaaggacagggtggggcggggtgcttagactgtgctacgggttccagaactgcaatccacaaaagccaaaccagcttgtttcaaccggggagtgccacctgcggagcaaggctgccctggccagggttcttgggaagcacctgcatgtatcaccatcctgcctctgaagagccctcagcatgaagcaagcatgaatggcggccaacctaaccaataaagttattttataagtgcatctgcaaggatggaatggttggtggcagccctcggcatcgcctcggggtacctagacacacaggccttgaaccgtgtcagctctttataaagagtagcctatataaaaactcaagtt

SEQ ID NO: 16 - protein, 376 aa, Mus musculus
MQWASVLLLAGLCSLSQGQYDEDSHWWIQYLRNQQSTYYDPYDPYPYEPSEPYPYGVEEGPAYAYGAPPPPEPRDCPQECDCPPNFPTAMYCDNRNLKYLPFVPSRMKYVYFQNNQISAIQEGVFDNATGLLWVALHGNQITSDKVGRKVFSKLRHLERLYLDHNNLTRMPGPLPRSLRELHLDHNQISRVPNNALEGLENLTALYLHHNEIQEVGSSMRGLRSLILLDLSYNHLRRVPDGLPSALEQLYLEHNNVYTVPDSYFRGSPKLLYVRLSHNSLTNNGLATNTFNSSSLLELDLSYNQLQKIPPVNTNLENLYLQGNRINEFSISSFCTVVDVMNFSKLQVLRLDGNEIKRSAMPVDAPLCLRLANLIEI

SEQ ID NO: 17 - DNA, 1723 bp, Mus musculus
  CDS (125)..(1093)
gacagttggaagatgacagttgaccagaattcctatccattggtcaggggcaaataccaaggactctgtatgctcagtcactctgtgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaatattaaatgtgtttgaaagatatatggtcttgtgaaaaagtatttcttataattgtagtgtgcaaataactatgtaatagcagtatacattccttcagtttctaatgacatctatcatctataagaacatcaactcattttgcaaacttggttagttgacagctacctgtattcctctacatacaaaaaataaaatatgagatatagagc

SEQ ID NO: 18 - protein, 322 aa, Mus musculus
MGMLARVALGLIIIDAVLAAPTTELFNYDSEVYDAILEDTGTFYNYEHIPDNHVENEKVSERLSGNRELLTPGPQLGDNQDEDKDEESTPRLIDGSSPQEPEFPGLLGPHTNEDFPTCLLCTCISTTVYCDDHELDAIPPLPKKTTYFYSRFNRIKKINKNDFASLNDLKRIDLTSNLISEIDEDAFRKLPHLQELVLRDNKIKQLPELPNTLTFIDISNNRLGRKGIKQEAFKDMYDLHHLYITDNSLDHIPLPLPESLRALHLQNNDILEMHEDTFCNVKNLTYVRKALEDIRLDGNPINLSRTPQAYMCLPRLPIGSFI

SEQ ID NO: 19 - DNA, 4909 bp, Mus musculus
  CDS (390)..(3296)
gcagctccgagctaggtgctatcgcaaggccagagcgcacagcccggcggagagagcagatccttgctcagatcgagtcaaatcggggccaaggcggaggacgaagagtccaggctcctattctggacttgttccccagctccgggggcgcttctaggtcctgcagcagccaggagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtccctttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggaccaggaagcataaggaagaagcgatttgtgtccagcccccgttatgtggaaaccatgctcgtggctgaccagtccatggccgacttccacggcagcggtctaaagcattaccttctaaccctgttctcggtggcagccaggttttacaagcatcccagcattaggaattcaattagcctggtggtggtgaagatcttggtcatatatgaggagcagaagggaccagaagttacctccaatgcagctctcacccttcggaatttctgcaactggcagaaacaacacaacagccccagtgaccgggatccagagcactatgacactgcaattctgttcaccagacaggatttatgtggctcccacacgtgtgacactctcgggatggcagatgttggaactgtatgtgaccccagcaggagctgctcagtcatagaagatgatggtttgcaagccgccttcaccacagcccacgaattgggccatgtgtttaacatgccgcacgatgatgctaagcactgtgccagcttgaatggtgtgactggcgattctcatctgatggcctcgatgctctccagcttagaccatagccagccctggtcaccttgcagtgcctacatggtcacgtccttcctagataatggacacggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaatggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggagggctgatacactgagtgctggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactgactagatacttcttatcacaaagattgggaaaggcaaagcagaaagatggtaagactgggtttcaaacaaggcttggtttctatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactataattcatgttgaggtagaaacaattcatctatttataaaatgtacattggaaaaaaaaaagtgaagtttatgaggtacacataaaaactgaaggaaacaatgagcaacatgcctcctgctttgcttcctcctgaggtaaacctgcctggcgattgaggttgtttaagattatccatggctcacaagaggcagtaaaataatacatgttgtgccagagttagaatggggtatagagatcagggtcccatgagatggggaacatggtgatcactcatctcacatgggaggctgctgcagggtagcaggtccactcctggcagctggtccaacagtcgtatcctggtgaatgtctgttcagctcttctactgagagagaatatgactgtttccatatgtatatgtatatagtaaaatatgttactatgaattgcatgtactttataagtattggtgtgtctgttccttctaagaaggactatagtttataataaatgcctataataacatatttatttttatacatttatttctaatgataaaacctttaagttatatcgcttttgtaaaagtgcatataaaaatagagtatttatacaatatatgttaactagaaataataaaagaacacttttgaatgtgtatgcctattttctggagtgggattaacttctgggcaagaaatctgatgagacacaaacattggacttcaagacagttttaaattttgggtaaatgaactgtatttcctgtttatagacgtactaataaaaaagaagttgatgatgtctttagtggtaagattgttactaatgtggttggcaaattgctgtaaagagccagatagtaagcatttatggcattgtaggctatctttcctgccacaaccatgtgacagtgagtgctttgtaggactgagagcagccataaatgacatgtaaatgataaactgtggctgtgctttaataaaactttatttacaaaaacaaaaaaaaaaaaaaaaaaaaaaaaaaaaaaaaaaaaaaaaaaaaaaaaaa

SEQ ID NO: 20 - protein, 968 aa, Mus musculus
MQPKVPLGSRKQKPCSDMGDVQRAARSRGSLSAHMLLLLLASITMLLCARGAHGRPTEEDEELVLPSLERAPGHDSTTTRLRLDAFGQQLHLKLQPDSGFLAPGFTLQTVGRSPGSEAQHLDPTGDLAHCFYSGTVNGDPGSAAALSLCEGVRGAFYLQGEEFFIQPAPGVATERLAPAVPEEESSARPQFHILRRRRRGSGGAKCGVMDDETLPTSDSRPESQNTRNQWPVRDPTPQDAGKPSGPGSIRKKRFVSSPRYVETMLVADQSMADFHGSGLKHYLLTLFSVAARFYKHPSIRNSISLVVVKILVIYEEQKGPEVTSNAALTLRNFCNWQKQHNSPSDRDPEHYDTAILFTRQDLCGSHTCDTLGMADVGTVCDPSRSCSVIEDDGLQAAFTTAHELGHVFNMPHDDAKHCASLNGVTGDSHLMASMLSSLDHSQPWSPCSAYMVTSFLDNGHGECLMDKPQNPIKLPSDLPGTLYDANRQCQFTFGEESKHCPDAASTCTTLWCTGTSGGLLVCQTKHFPWADGTSCGEGKWCVSGKCVNKTDMKHFATPVHGSWGPWGPWGDCSRTCGGGVQYTMRECDNPVPKNGGKYCEGKRVRYRSCNIEDCPDNNGKTFREEQCEAHNEFSKASFGNEPTVEWTPKYAGVSPKDRCKLTCEAKGIGYFFVLQPKVVDGTPCSPDSTSVCVQGQCVKAGCDRIIDSKKKFDKCGVCGGNGSTCKKMSGIVTSTRPGYHDIVTIPAGATNIEVKHRNQRGSRNNGSFLAIRAADGTYILNGNFTLSTLEQDLTYKGTVLRYSGSSAALERIRSFSPLKEPLTIQVLMVGHALRPKIKFTYFMKKKTESFNAIPTFSEWVIEEWGECSKTCGSGWQRRVVQCRDINGHPASECAKEVKPASTRPCADLPCPHWQVGDWSPCSKTCGKGYKKRTLKCVSHDGGVLSNESCDPLKKPKHYIDFCTLTQCS

SEQ ID NO: 21 - DNA, 3673 bp, Mus musculus
  CDS (392)..(2929)
ggggagaacccggggaagacccacagatacacagaaacgagagagacagaagaggagagagacagagacaaagagacagcagcaggaggaggaggaggaggaggcaaagacagggcgagcactgaggcaaagccagacagctgagcagagggagaagccaaggagtcacagacaaagacctgggacagaaaggcaaaggaggaagttttggagcagaaagaacccgtgggcacttttcctgagcccaggggctgttttctttttcatcttctctgaaagtccctagcccatttaaaaactttgcctcagattttggcacaagccccaaagcccttctggctttaacgaggagccttccacagttagggtgtggagcattttggtgccgcagatggcctcaatccatcccagctgcagcccgggtaccatgtcccagatgggcttgcatcccaggaggggcttgactgggcactggctgcaaagattccaaccctgcttgccgcttcacactgtgcagtggcggaggctgctgctgctggccttcctcctgtccttagcgtggcccgccagccccctcccccgggaggaggagatcgtgtttccagagaagctcaatggcagtagcatcctacctggatcaggcgttcctgccaggctgctgtaccgattgccagcctttggggagatgttgctactagaactagaacaggaccctggggtgcaggtagagggtttgactgtacagtacctgggccaggcacctgagatgctgggtggggcagagccaggtacctacctgactggcaccatcaatggagatccggagtcggtggcatctctgcactgggacgggggagccctattaggggtactgcagtaccgtggggccgaactccacctccagcctctggaaggaggcgcccttaactctgctgggggaccgggggctcacatcctacgccggaagagtcctgccagcagccaaggtcccatgtgcaccgtcaaggctccttctgggagcccgagtcccatttcccgcagaaccaagcgcttcgcttctctgagtagattcgtggagacactggtggtagcagatgacaagatggcagcattccatggtacagggttaaagcgctacctgctgacggttatggcagctgccgctaaagcctttaaacacccaagcatccgaaaccctgtcaacttggtggtgacgcgcctggtgatcctggggtccggccaggaagggccccaagtggggccaagtgccgcccagaccctacgcagcttctgcacctggcagcggggcctcaacacccctaacgactcagatcctgaccactttgacacagccattctgttcacccggcaggacctgtgtggggtctccacttgtgacaccctgggtatggctgatgtgggcacagtgtgtgatccagctaggagctgtgctattgtggaagatgatgggctccagtcagccttcactgctgctcatgaactgggccatgtcttcaacatgctccatgataactccaagccatgcactaacttgaatgggcaggggggttcctctcgccatgtcatggctcctgtcatggcccatgtggaccctgaagagccctggtcgccctgcagtgcccgattcatcactgacttcctggacaatggttatgggcactgcctcttagacaaaccggaggctcccctccatctaccagcgacttttcctggcaaggactatgacgctgaccgccaatgccaactgaccttcggtcctgactcaagccattgtccacagctgccaccgccctgtgctgccctctggtgctctggccacctcaatggccatgccatgtgccagacgaagcactcaccttgggctgatggcactccctgcgggtcttcacaggcctgcatgggtggccgctgtctgcacgtggaccagctcaaggacttcaatgttcctcaggctggaggctggggcccctggggaccatggggtgactgctccaggacttgtgggggtggtgtccagttctcctcccgggattgcacgaggcccgtcccccggaacggtggcaagtattgtgagggccgccggactcgcttccgctcctgcaacacggagaactgcccacacggctcagcattgaccttccgtgaagagcagtgtgctgcctacaaccaccgaaccgacctcttcaagagctttccagggcccatggactgggttccgcgctacacaggtgtggcccctcgagaccaatgcaaactcacctgccaggcccgggcactgggctactactacgtattggagccccgggtggcagatgggactccctgctccccagacacctcctctgtctgtgtccagggccgctgtatccatgctggctgtgaccggatcattggctccaaaaagaaatttgacaagtgcatggtgtgcggcggggatggctctcgctgcagcaagcagtcgggctccttcaaaaaattcaggtatggatacagcgatgtggtcacgatccctgcgggggccacccatatccttgtacggcagcagggggggtctggtctcaagagcatctacctggccctgaagctttctgacggttcttacgccctcaatggtgaatacacgctgatgccctccccaacagatgtggttcttcctggggcagtcagcttgcgctacagcggagccacagcagcctcagagacactgtctggacatgggccgctggcccagcccttgacgctgcaagtcctggtggctggcaacccacagaatgcacgtctgcggtacagtttctttgtcccgcggccagtcccttcaacaccacgccctcctccccaagactggctgcaacgcagggcagagatactgaagatccttcggaagcgtccctgggcaggccggaaataacctcactgtcccggctgccctttttgggcgccggggcctcggactcatctgggagaatgagcaggcttctgcaactgcctcctgctaaaacacagtagggaggtgtagagggtgagatctgcctgcctcactgccccaaaccgcaggctggccctgccctggcttcctgccctgggaggcagtgatgtcttggtgaatggaaaggggctaggtgacagtaccctatctactaaactgccccctctaccctgcaggtcacaggaggaatggggggaagacagggtgggtcctgggccctagttgtatttatttggtatttattcatttttatttagcaccaggaaaggggactagggtcttggggaaactcacctattatagccctaacctagctatgaaatccagggtgttggtgacaaatatgagtggtgtgtgtgtgtgtgtgtgtgtgtgtgtgtgtgtttatgtatgaggtacaacctgccctgctttcctctccctaatttttttttttttctgggaaaaggaaagtcaaaggtaggactgccttcagggagtaagggatgattgtgtttttaaattgaagtttgctatttatatgctctttttggagtcagacaaatgtgggttatattctggccccgcatctttgagcattagttttctcatgtgccaataataatcccttagaaattggttgtaaggattaaatgatgttaataaagaactagcatagagcctctc

SEQ ID NO: 22 - protein, 845 aa, Mus musculus
MASIHPSCSPGTMSQMGLHPRRGLTGHWLQRFQPCLPLHTVQWRRLLLLAFLLSLAWPASPLPREEEIVFPEKLNGSSILPGSGVPARLLYRLPAFGEMLLLELEQDPGVQVEGLTVQYLGQAPEMLGGAEPGTYLTGTINGDPESVASLHWDGGALLGVLQYRGAELHLQPLEGGALNSAGGPGAHILRRKSPASSQGPMCTVKAPSGSPSPISRRTKRFASLSRFVETLVVADDKMAAFHGTGLKRYLLTVMAAAAKAFKHPSIRNPVNLVVTRLVILGSGQEGPQVGPSAAQTLRSFCTWQRGLNTPNDSDPDHFDTAILFTRQDLCGVSTCDTLGMADVGTVCDPARSCAIVEDDGLQSAFTAAHELGHVFNMLHDNSKPCTNLNGQGGSSRHVMAPVMAHVDPEEPWSPCSARFITDFLDNGYGHCLLDKPEAPLHLPATFPGKDYDADRQCQLTFGPDSSHCPQLPPPCAALWCSGHLNGHAMCQTKHSPWADGTPCGSSQACMGGRCLHVDQLKDFNVPQAGGWGPWGPWGDCSRTCGGGVQFSSRDCTRPVPRNGGKYCEGRRTRFRSCNTENCPHGSALTFREEQCAAYNHRTDLFKSFPGPMDWVPRYTGVAPRDQCKLTCQARALGYYYVLEPRVADGTPCSPDTSSVCVQGRCIHAGCDRIIGSKKKFDKCMVCGGDGSRCSKQSGSFKKFRYGYSDVVTIPAGATHILVRQQGGSGLKSIYLALKLSDGSYALNGEYTLMPSPTDVVLPGAVSLRYSGATAASETLSGHGPLAQPLTLQVLVAGNPQNARLRYSFFVPRPVPSTPRPPPQDWLQRRAEILKILRKRPWAGRK

SEQ ID NO: 23 - DNA, 3002 bp, Mus musculus
  CDS (18)..(2810)
ccggcgggcagcgcactatgcggctcgagtgggcgtccttgttgctgctactgctgctgctgagcgcgtcctgcctgtccctggccgctgacagccccgccgcggcacctgcccaggataaaaccaggcagcctcaggctgcagcagcggccgccgagccggaccagccgcagggggaggaaacacgggagcgaggccatttacaacccttggccgggcagcgcaggagcggcgggctggtccataatatagaccaactctactctggcggtggcaaagtgggctaccttgtctacgcgggcggccggaggttcctgctggacctggagagagatgacacagtgggtgctgctggtagcatcgttactgcaggaggagggctgagcgcatcctctggccaccggggtcactgtttctacagaggcaccgtggacggcagccctcgatccctagctgtctttgacctctgcgggggtctcgatggcttctttgcagtcaagcatgcgcgctacactctaaagccactcctgcgtgggtcctgggcagagtatgaacgaatttatggggatggatcttcccgcatcctgcatgtctacaaccgcgagggctttagcttcgaggccctgccgccacgcgccagttgcgagactcctgcatccccatctgggccccaagagagcccctcggtgcacagtagatctaggagacgctcagcgctggccccgcagctgctggaccactcagctttctcgccatctgggaacgcgggacctcagacttggtggaggcgtaggcgccgttccatctccagggcccgccaggtggagctcctcttggtggctgactcgtccatggccaggatgtatgggcggggcctgcagcattacctgctgaccctggcctccatcgccaacaggctgtacagtcatgcaagcattgagaaccacatccgcctggcggtggtgaaggtggtggtgctgacggacaaggacacgagtctggaggtgagcaagaatgcggccacgaccctcaagaacttttgcaaatggcagcaccaacataaccagctaggggatgatcacgaagagcactacgatgcagccatcctgttcacccgagaggatttatgtgggcatcattcatgtgacaccctgggaatggcagacgttgggaccatatgttctccggagcgcagctgtgcagtgattgaagatgatggcctccatgcagccttcactgtggctcatgaaattgggcatctacttggcctttctcatgacgattccaaattctgtgaagagaacttcggtactacagaagacaagcgtttaatgtcttcaatccttaccagcatcgatgcatccaagccctggtccaaatgcacgtcagccaccatcacagaattcctggatgatggtcatggtaattgtttgctagacctaccacggaagcagattttgggtcccgaggaactcccaggacagacctacgatgccacccagcagtgcaacttgacatttgggcctgagtactcggtgtgccctggcatggatgtctgtgcgcggctgtggtgtgctgtggtgcgccaaggccaaatggtgtgtctgaccaagaagctgccggctgtggagggcactccctgtgggaagggaagagtctgccttcaaggcaaatgtgtggacaaaactaagaaaaaatattactcgacatcaagccatggaaattgggggtcctggggcccctggggtcagtgttctcgctcatgcgggggaggagtgcagtttgcctaccgccattgtaataaccctgcacctcgaaacagtggccgctactgcacagggaagagggccatataccgttcctgcagtgttacaccctgcccacccaatggtaaatcttttcgccatgagcagtgtgaagccaaaaatggctatcagtctgatgcaaaaggagtcaaaacatttgtagaatgggttcccaaatatgcaggtgtcctgccggcagatgtgtgcaagcttacctgcagagctaagggcacaggctactatgtggtcttttctccaaaggttacggatgggactgaatgcaggccgtacagcaactctgtgtgtgtccgaggacggtgtgtgagaactggatgtgacggcattattggctcaaagctacaatatgacaagtgtggagtgtgcggaggggataactccagttgtacaaagattatcggaaccttcaataaaaaaagcaagggttatactgacgttgtgaggatccctgaaggagcaacccacataaaagtccgacagttcaaagccaaagaccagactagattccctgcctacttagccctgaagaagaaaactggcgagtaccttatcaatggcaagtacatgatttccacttcagagaccatcatcgacatcaatggtaccgtcatgaactacagtggatggagccacagagatgattttttacatgggatgggctattcagccacaaaagaaatcctgatcgtgcagatccttgccacagacccaactaaagcgctaggcgtccgttacagcttttttgttcccaagaagaccactcaaaaagtaaactctgtcatcagccatggcagcaacaaggtgggaccacactctacacagctgcagtgggtgacaggtccatggctggcctgctccaggacctgtgacacaggctggcacactaggaccgtgcagtgccaggatggaaacaggaaattagctaaaggatgccttctctctcagaggccttctgcatttaagcaatgtctgctgaagaaatgttagcctgtggtttactctaatgcacaaaaaaacaacaggaggatcatcgcagatacagctgtggtgaagacaaggcctacccaaagcacagaaagtcatgccttcatgtcattgtcaccacgagtcgaattatgggcagaatctgctctctgcgaccaaaaggtttactctacttggtgaatgatggtaccgtga

SEQ ID NO: 24 - protein, 930 aa, Mus musculus
MRLEWASLLLLLLLLSASCLSLAADSPAAAPAQDKTRQPQAAAAAAEPDQPQGEETRERGHLQPLAGQRRSGGLVHNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFPAYLALKKKTGEYLINGKYMISTSETIIDINGTVMNYSGWSHRDDFLHGMGYSATKEILIVQILATDPTKALGVRYSFFVPKKTTQKVNSVISHGSNKVGPHSTQLQWVTGPWLACSRTCDTGWHTRTVQCQDGNRKLAKGCLLSQRPSAFKQCLLKKC

SEQ ID NO: 25 - DNA, 3077 bp, Mus musculus
  CDS (59)..(2200)
ccacgcgtccggccagccccagggaccgcagcaatctcccaaactccccttccccaggatgacagaggagttaatcacccctgtgtactgcaccggggtgtcagcccaagtacagaagaagcgggacaaggagctgggcttgggccgccatgaaaacgccatcaagtacctgggccaggattatgaaacgcttcgggcaagatgcctgcagagtggggtcctcttccaagacgaggccttccctccggtttctcatagcctgggcttcaaggaactgggtcctcattcctctaaaacctatggcatcaaatggaagcggcctacggaactgatgtcaaacccccagttcatcgtggatggagccacccggacagacatctgccagggagcactgggggactgttggctcctggctgccattgcctccctcaccctcaacgagactattctgcaccgagtggttccctacggccagagctttcaggatggctacgctggcatctttcatttccagctgtggcagttcggggagtgggtcgacgtggtcatagatgacttgctgcccaccaaggacgggaagctggtgttcgtgcactctgcccagggcaacgagttctggagcgcactgctggagaaagcctatgctaaagtgaatggcagctatgaggctctttcgggaggctgcacctcagaggccttcgaggactttaccggtggggtcactgagtggtacgacctgcagaaggcccccagcgacctctaccagatcattctaaaggccctggaacgaggctccttgctgggctgctccattaatatctccgatatccgtgatttagaggctattacttttaagaacctggtgaggggccatgcgtactctgtgacgggcgccaagcaggtaacttaccagggccagcgggtgaacctaatccggatgcggaacccctggggtgaagtggagtggaaaggaccctggagtgacagctcctatgagtggaacaaagtggacccctatgaacgagagcagctgagggtcaagatggaggatggggagttctggatgtcgttccgagacttcatccgtgaattcaccaaactggaaatctgcaaccttacaccggacgcccttaagagcaggaccctccggaattggaataccacattttacgagggcacctggcgtcggggaagcaccgctggaggctgcaggaactacccagctaccttctgggtaaacccccagttcaagatccggttggaggaggtggatgacgcagacgactatgacaaccgggagtcgggctgcagcttcttgctggccctcatgcagaaacaccgccgcagggagcgtcgctttggccgggacatggagaccatcggttttgcagtgtaccaggtccctcgggagctggcgggtcagcctgtgcacttgaagcgtgatttcttcctggccaacgcttctcgggcgcagtcagagcacttcatcaaccttcgggaagtcagcaaccgtatccgcctgccgcccggggagtacatagtggtgccctccaccttcgagcccaacaaagaaggcgacttcctgctgcgcttcttctcagagaagaaggctgggacccaggaactagatgaccagatccaggccaacctccctgatgagaaagttctctctgaagaggagattgatgacaacttcaagaccttgttcagcaagttggcaggggatgacatggagatcagcgtcaaggagctacagaccatcttgaacaggatcattagcaaacacaaagacctgcgcactaatggcttcagcctggagtcgtgccgcagcatggtgaacctcatggatcgagatggcaacgggaagctgggtctggtggagttcaacatcctgtggaaccgcatccgaaattacctgaccatcttccggaagtttgacctggacaagtctggcagcatgagtgcctatgagatgaggatggccatcgaggctgcaggcttcaagcttaacaagaagctgcatgaactcataatcacccgctactcggagcccgacctggccgtggactttgacaactttgtgtgctgtcttgtgcggctggagaccatgttccggtttttcaaacttctggacacagacctggatggtgttgtgacctttgatctatttaagtggctccagctgactatgtttgcctgaagtggggaatcgctccgctccagttgcctttgttcttccattcttcctctgccaagccacgcctccccgccaagccacgcctccccgccgtgccactccagactacaccagcttctggtgccttcctcagaacaggaaggaaggcacccttgtcctcctgttccccttgtcctcctgtcctttcacctccccactacctctgttcgactgctctgggcagtcacggggccctccctgcccctgtagtcagactgggcaccctgacccctgctcagggctaagaccaggaagacagctccctgctcatccctgcctcggggctggggcaggggcaggggcaggggcaggggcaggcacccctgcccctccccctcgctgctactgtccggatgtgagtcttgcctaatctgtggaggctctagcccctgtcatcccctagagctgcagacttcataaccactagctagctccctgtctgcacccgggtgttgaaggcctcacctcactgtgggagccccacagccagactcaccttcctgtgccttctgtgcagcagccaagatccctctgcaaaccaaactacacctctcctagcctgtgtgcccaccaggaacttcctggccccgagggtgctcaggccctccccatccccgacccctctccctctctttttttgtatatatactggtcattttcaagggaatgcctgagggatgagtgtactggttatggaggaactgggggtggggtagggaggtcccgtgtttcagtgcagtgagaaaataaaatgaaaggctgtaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - protein, 713 aa, Mus musculus
MTEELITPVYCTGVSAQVQKKRDKELGLGRHENAIKYLGQDYETLRARCLQSGVLFQDEAFPPVSHSLGFKELGPHSSKTYGIKWKRPTELMSNPQFIVDGATRTDICQGALGDCWLLAAIASLTLNETILHRVVPYGQSFQDGYAGIFHFQLWQFGEWVDVVIDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTGAKQVTYQGQRVNLIRMRNPWGEVEWKGPWSDSSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDNRESGCSFLLALMQKHRRRERRFGRDMETIGFAVYQVPRELAGQPVHLKRDFFLANASRAQSEHFINLREVSNRIRLPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKLLDTDLDGVVTFDLFKWLQLTMFA

SEQ ID NO: 27 - DNA, 2105 bp, Mus musculus
  CDS (418)..(1671)
gagccttcccggcgcgtgagccggatccggtggcaccgcggggaagagacaggaccgggcggtggcggcagagacagggggacgcacccggtgcagaagatccaataggagcacgccgccgcaacctctcccgcgcgctccggtcgccgactctacgccgatcgcccactccccgcaccttggactacaccgggaaaaagacgcagcctggcccgaactggggcggagatccttcttgcttcccaagagctcgggagggaagattctcccgccgccgagaaccccgccggactggaggaacccgtggcctggagagcgctggctgtgccagaccccagcctgatggatgtctggtgtggataatgagggaagaacgtgccttttacacccaagaggtgaccccggagcgtgccccggatgaccccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtgctcatggtgctggcagggttagcctcatccacggcagatatccaacacaacaccgtccactatggcagcgcccttaagcgcctggatacttttgaccggcagggcatagtgcaccgcctcagtacctacaccaagatgctctttgtccgggaaccctttgagcggctggtctctgctttccgagacaagtttgagcatcctaacagctactatcatcctgtctttggcaaggctatcctggcccggtaccgcgccaacgcctcgcgggaggcactgcggactggctccggtgtgcagttccccgagttcgtccagtacctgttggatgtccaccggcccgtgggcatggacatccactgggaccatgttagccggctgtgcagcccctgcctcatcgactatgactttgtgggcaagttcgagagcatggaagacgatgccaacttcttcctgcgtctcatccatgcgcccgggaacctgactttcccgaggttcaaggacaggcactccgaggaggcgcggaccacatcgagaatcacccatcagtacttcgctcagctctcctcgctgcagcgacagcgaacctacgacttctactacatggattacctgatgttcaactactccaaacctttctcggacctgtactgagggcggggcctgctggtcaggggcggggtctgccggtcatgcccactcacctgcgcataggcggcctccggggactgagctctgaggatgtgaggccttgtggctgtggccctagggtgggccacagaggcccagacaatggaccttgacccttgtcccacacccatttcctcattgggttggctgagtttgagacggagcacgactcggatggatgctttaagaactcagctgagctatgctgagctatgctgtccagggaagcctgagacccagaagagggccccagcgtcgagggatgtcctacatccccttatcctttgccttgtaccaaaccacgtggtttgctgcttttctatgacccagggtcatctgaataaagcacatggttttcagagcaaaaaaaaaaaaaaaaaaaaaaaaaaaaaaaaaaa

SEQ ID NO: 28 - protein, 417 aa, Mus musculus
MTPQLGTMRLACMFSSILLFGAAGLLLFISLQDPIELSPQQVPGIKFSIRPQQPQHDSHLRISTEKGTRDSPSGSPRGLQLQAPDQPRPHPKAAGSPLRLRQRRRRLLIKKMPAAGTNQGNNSSETFIQPRPRTMDSRWVSLHQTQQERKRVMREACAKYRASSSRRAVTPRHVSRIFVEDRHRVLYCEVPKAGCSNWKRVLMVLAGLASSTADIQHNTVHYGSALKRLDTFDRQGIVHRLSTYTKMLFVREPFERLVSAFRDKFEHPNSYYHPVFGKAILARYRANASREALRTGSGVQFPEFVQYLLDVHRPVGMDIHWDHVSRLCSPCLIDYDFVGKFESMEDDANFFLRLIHAPGNLTFPRFKDRHSEEARTTSRITHQYFAQLSSLQRQRTYDFYYMDYLMFNYSKPFSDLY

SEQ ID NO: 29 - DNA, 1369 bp, Mus musculus
  CDS (40)..(1281)
attcagacatgaagagagacacagtggtctgaagtggtcatgaaagccaaacaagtcttcttttctgtcctgctgtttgggacagcagggcttctgctcttcatgtacttgcaagcatggattgaagaacatcatacagggaaaatagagaagaaaagggatcagaaaggagtatcggtgactacgggaaaaatccagaaacagatcacgaatcagaactctgaggttcacatgcctgaagatctgaagaagaaagggggagatctgctcaacctagggagtccaacaagggttttaaggaagatcagccattcacaaagggagaacggagcttacagatcaactgaagcacatcaaggagctaaaattgaagtttttcagaaacccatccagatggactggccactggtcactcagcccttaaacaaaagtttggtccaaggcaacaaatggaagaaagcagatgcaacccaagagaagcgtcggtcattccttcatgagttttgcaagaaatatggtagagtaaatgatcccaagttcaacctttttcatatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttgcattcatttttctaaagccctacatagatttaatgatgatggcctcaaataagctactgtaattgtcctacaattctctgtatg

SEQ ID NO: 30 - protein, 413 aa, Mus musculus
MKAKQVFFSVLLFGTAGLLLFMYLQAWIEEHHTGKIEKKRDQKGVSVTTGKIQKQITNQNSEVHMPEDLKKKGGDLLNLGSPTRVLRKISHSQRENGAYRSTEAHQGAKIEVFQKPIQMDWPLVTQPLNKSLVQGNKWKKADATQEKRRSFLHEFCKKYGRVNDPKFNLFHIVSRIYVEDKHKILYCEVPKAGCSNWKRILMVLNGLASSAYNISHDTVHYGKHLKTLDSFDLKGVHMRLNTYTKAVFVRDPMERLVSAFRDKFEHPNSYYHPVFGKAIIKKYRPNASAEALNNGSGVKFKEFAYYLLDAHRPVGMDIHWERVSKLCYPCLINYDFVGKFETLGEDANYFLQLIGAPKELTFPNFKDRHSSDERTNAHVVRQYLKDLSTAERQLIYDFYHLDYLMFNYTTPHL

SEQ ID NO: 31 - DNA, 4264 bp, Mus musculus
  CDS (561)..(2246)
ggagtcaggttgacgcgccctgggtgcggggtgctaggcgcggggctgggcgcgcgctcccgcggagtcggagctgcagaggcttccgagatcgtcttgggcttttcaggtcacctagcattggtatgaatctgctcctcaagaagagaaaatattctgaaggcatgtccgtttacctgtcaacgaggaacccataatgtcttctcatctccagtgtggctccatgctctctcctgatgctactccaactttctttggtgccatgggaaaaacctgaagcacgagcagagtcacctccaaatctgtgctcacttcattttgttgcatggctggtgcggaacagactattggaatttgctcttgtgtgttgcccttcgtacctgaaccaaagactgtgcctcattctctagtgcattgctgcctgctgtcttcctcgttcatttctgttgcttgtgccttcctactgtcttctgtcatcacaggacacccttaaagtttctcactggactcctcgagggctggcccatggctctctaagccagctgtccaaggtctctaccatgaggcattgcattaattgctgcgtccagctgttccccgaagacacacacaaacagcaagttgcctgccaaggaggcccccatcacagtcatcaggcgtgccccacttgcaaaggagaaaacaaaattctgtttcgtgtggacagtaagcagatgaacttgcttgctgttctcgaagtgaggactgagggcaatgaaaactggggtgggtttttgcgcttcaggaaggggaagcgatgtagcctggtctttggattgataataatgaccttggtgatggcttcttacatcctttccggggctcatcaggaacttctgatttcctcccctttccattatgggggctttcccagcaaccccagcgtgatggatggtgagaaccccagtgatgtgaaagagcatcactaccagccttctgtaaataacatctcctatgtaaaggactatccgagcattaaactgattatcgacagcattgctgccaggattgagttcacaaccaggcagctcccggacttacaagatctcaagagacaagagttgcatatgttttctgtaatccccagcaaattcctcccgactagcaagagcccttgttggtatgaggagttctcgggcaggaacaccactgacccctacctgaccaattcctacgtgctctactccaagcggttccgctctacctttgacgctctgcgcaaggttttctggggccacctgtcccacgtgcagggcaagcacttccgcctgcgctgcctgccccacttctacatcattgggcagcccaagtgtggaaccactgacctgtacgaccgcctccggctgcatccagaagtgaaattctcagccatcaaggagccgcactggtggacccggaagcgctttggaattgtccgcctgcgggacggactacgagaccgctaccctgtggaagattacctggacctctttgacttggctgcacaccagatccatcaaggactgcaggctgcctctgcagagcagcccagcaagatgaataagatcattattggagaggccagcgcctctacaatgtgggataacaatgcctggaccttcttctatgacaacagcacagacggcgagccaccattcctgacccaagacttcatccatgcctttcaaccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccactgactctgtcattgccatgactttaaaagtctccaggcggagaactgtcacccacagcgtggagaaagccgctggagccccacccaatcctcttctcatgtccaaggcacctggtgtgggaactattttaaagctttccttgccatgttcagcagctctgctcttgattgcaaggctcgtccacctgaatccttggaaacgacactgggggtaggcttctgtctgttcccagagtgccttggatctcaggagggcagtggatctctggcacccagtgggtaggaagaggaagctacgggaggccatagaccagccttgccttccttcctccaggagagtttgtctgggtgtagggttggacagctctgggatccgattaaggccatgttctggggctgcccctcacgtgttatatcccagcaggccttgcataaatcgacaaccccatgtggacagagaacagagctgcttggagaatggaggctctttcgtttttgttcgtattcccaagtcagcccttcggtgacatcagaaatgagccctgttccctttgctgtgctcagaagaaactcagaagtcagcttttggcagtaggctcgtctctctgacctattctgcccatacactttgtttatgcccagcgtagagaaggagcgttttaaaacaaactgcttctcgcatttgactggccctgcctctcttttctgctcccttggatgttttccttaaggacttcagtttatatttggaaagggaagttttgctgttaatgccagaacagaacacctcaaggcatcgaacagtggaaaaggaaggaagtgaagccctccccatttgtgggggtcactggagagtgtggggatgtttttggttctggctggttacaggctcccccctgtcttcctgttggaggaatctttatccacattctctgtcatgtgcagccaaagttctgtaactcactaagggtgagtctcaagacacccttagaatgtcctcagaaacacatctcacaaactaagctcgggccacatgtggtgtcactaggagatagctttgatttttatgaacctttccaggcctgctagcagcttggttcgcaaaggatatgctgaaagtcttgtcattttaaaaaaaaaaaaagaccccagggaataccagagagaacaacttaaacccacttggcacacaggacaaggtggggatgtcacaaagcttttcattggagcgttgccaagggatgctgcctgtgaagctgtgttctcaggtcaggatcctgttcaccagcgtcagaaagtgtacacaccacatgcccgactgtcacttggacgtgtgagaaaagctgggctttggcagcaggaggctgtgcagttgctcgactgtgtgagttctgctgcggtccacccttgttggagatgggactctcagctcattggaaggttccgggagagctctgggcatcagcatggactgggctcgttctgctactgaaggggctgtgtgtcatgtggcctgtgttccaggacaattctcccttctctcacggtggccacttggctcggcagccagtgggaaaatctagcctgagtacaaggatgctttgggaagaaatgacgagagagagagagagagagagagagagagagagagagagagagagagcgagagagagcgcaagctcaagcgcaagcgtgtgagaaagagcacgagagagtatttatttggaataccgtgtgtgtgtgtgtgtgtgtgtgtgtgtgtgtgtgtgtgtgtgtgtgtgcgtgcgcgcgcgagtgtgcgcgcgaacactcgtgcttatactgagcagggccctctcagctgtcttgttgatggggcttctcctccattagagacatgatgcctttatgaattaatttctccaacccttcgcctgtcatttccaggacatatcttactgccccacaccaaccacgactctaagagattttttctgacgcactccc

SEQ ID NO: 32 - protein, 561 aa, Mus musculus
MRHCINCCVQLFPEDTHKQQVACQGGPHHSHQACPTCKGENKILFRVDSKQMNLLAVLEVRTEGNENWGGFLRFRKGKRCSLVFGLIIMTLVMASYILSGAHQELLISSPFHYGGFPSNPSVMDGENPSDVKEHHYQPSVNNISYVKDYPSIKLIIDSIAARIEFTTRQLPDLQDLKRQELHMFSVIPSKFLPTSKSPCWYEEFSGRNTTDPYLTNSYVLYSKRFRSTFDALRKVFWGHLSHVQGKHFRLRCLPHFYIIGQPKCGTTDLYDRLRLHPEVKFSAIKEPHWWTRKRFGIVRLRDGLRDRYPVEDYLDLFDLAAHQIHQGLQAASAEQPSKMNKIIIGEASASTMWDNNAWTFFYDNSTDGEPPFLTQDFIHAFQPEAKLIVMLRDPVERLYSDYLYFASSNKSADDFHEKVTEALQLFENCMLDYSLRACVYNNTLNNAMPVRLQVGLYAVYLLDWLTVFSKEQFLILRLEDHASNVKYTMHKVFQFLNLGPLSEKQEALMTKSPASNTRRPEDRSLGPMWPITQKILREFYGPFNTRLAQVLDDEAFAWKTT

SEQ ID NO: 33 - DNA, 1718 bp, Mus musculus
  CDS (374)..(1564)
acaaagcccggggggtccacgctggcgctggaggcgagccgggagcgagcccggggcgcggcgggacttggggcgcagggctgctacgggacgcggggctgctagcaggacgcgcgaccaggcgtgggttgcgatcgccacccccacggcggggcccgggacattttgggccctttggggccgcagcgagcggtggggaccaagacgagcagagtaccctgagcccggcaacttcggcccctccccgcccccacccggctgccctcagcgcgtccctctccatgtgcagacagccggctaggcgctccggctggcggcggatgggtgacctcttcctggccggggcgggctgttcgaggcggcggagccggcgatgaagaagaagcagcagcagcatcccggcggcggcacggatccctggccccatggggcccctgtggggggcgcccctccgtgcctgggcagctgcaagcgccggatcccgctgctgcctttcctgcgcttctccctccgggactacggtttctgcatggctaccctggtggtcttctgcctgggctccctcttctaccagctcagcgggggaccccctcgcttcctgctcgacctgcggcagtatttgggaaattccacttacttggatgaccatggaccacctcctagtaaggtccttcccttccccagccaggtggtgtacaacagagttggcaagtgtggcagtcgtaccgtggtcttgcttctgagaatcttgtcagagaagcacggcttcaatttggtcacatcagacattcacaacaagacccggcttactaaaaatgaacaaatggaactgattaagaatataagtaccgccgaacagccctatttattcacgagacatgtccacttcctcaacttctccaggtttggaggagaccagcctgtctacatcaacatcatcagagaccccgtcagtagatttctgtctaactatttcttccgtcgctttggagattggagaggggaacagaatcacatgatccgcacccccagcatgaggcaggaggagcgctacctggacatcaatgagtgcattctggaaaactaccctgagtgttccaaccccaggctgttttacatcatcccatatttctgtggacagcaccccagatgtagggagcccggcgagtgggcgctggagcgggccaagctgaacgtgaacgaaaacttcctgctcgtggggatcctggaggagctggaagatgtgctgctcttactggaaagatttttacctcattacttcaagggtgtgctgagcatctacaaggacccagagcacaggaaagcttggcaacatgactgtgaccgtgaggaagactgtcccctcccccgaggcggtgcagattctctaccagcgaatgagatacgagtacgagttctaccactacgtcagagaacaattccacctgctcaagcgcaagcttggactgaagtctcgcgtgagcggacctcccgtgagaccacagttcttcatccccacgcccctggagacggaggagccaatcgacgacgaggagcaagatgacgaaaaatggctagaagatatttataagaggtgatacctgcgccagattgtggcttctgtggcctcatccccccttttacaaagttctttggggaagagaaattctgaagggactttcattaatgctcgagtgcattaaaaagaacaaaacatt

SEQ ID NO: 34 - protein, 396 aa, Mus musculus
MKKKQQQHPGGGTDPWPHGAPVGGAPPCLGSCKRRIPLLPFLRFSLRDYGFCMATLVVFCLGSLFYQLSGGPPRFLLDLRQYLGNSTYLDDHGPPPSKVLPFPSQVVYNRVGKCGSRTVVLLLRILSEKHGFNLVTSDIHNKTRLTKNEQMELIKNISTAEQPYLFTRHVHFLNFSRFGGDQPVYINIIRDPVSRFLSNYFFRRFGDWRGEQNHMIRTPSMRQEERYLDINECILENYPECSNPRLFYIIPYFCGQHPRCREPGEWALERAKLNVNENFLLVGILEELEDVLLLLERFLPHYFKGVLSIYKDPEHRKAWQHDCDREEDCPLPRGGADSLPANEIRVRVLPLRQRTIPPAQAQAWTEVSRERTSRETTVLHPHAPGDGGANRRRGAR

SEQ ID NO: 35 - DNA, 5091 bp, Mus musculus
  CDS (318)..(1595)
ggcggcacccaaacagctaccccgtgcggagacccaaactttctactgccacttggagtctcgcggccgccgcctccgccccgcgcgtccggggcctgcccgtcagtccgtcggtccgcgtggagcagctcgggcgccgccgtgctcccgatccccgcagctgcagcgccgcagtcctggcccggacgcccgggcaagttctccagagttaaaagcgaagttcgggcgaggcgcgggccgagctgcctctgagcgcccccggcgtccccagtgcgcccagccccgccgggggcgccggtgacccttcggtgccagccatgtcgtccatcctgcccttcacccccccgatcgtgaagcgcctgctgggttggaagaagggcgagcagaacgggcaggaggagaagtggtgcgagaaggcggtcaagagcttggtgaagaagctcaagaagacggggcagttggacgagctggagaaggccatcaccacgcagaacgtgaacaccaagtgcattaccatccccaggtcactggatggtcggctgcaggtgtcccatcggaaggggctccctcacgttatctactgccgcctgtggcgatggcccgacctgcacagccaccatgaattacgggccatggagctctgtgagtttgccttcaacatgaagaaggatgaagtgtgtgtaaatccttaccactatcagagagtagagacgccagttctacctccagtgttggtgccacgccacaccgagatcccggccgagttccccccactggatgactacagccattccattcccgagaacactaacttccctgctggcattgagccccagagcaatattccagaaaccccacctcctggctacctgagtgaagatggagaaaccagtgaccaccagatgaaccacagcatggacgcaggttctccaaacctctccccgaatccgatgtccccagcacacaataacttggacctacagccagtcacctactgtgagccggccttctggtgctccatctcctactacgagctgaaccagcgagttggggagacattccacgcctcacagccatccatgacagtagatggcttcactgacccctccaactcggagcgcttctgcctgggcctactgtccaatgtcaaccggaatgcagccgtggaacttacaaggcgacacattgggagaggtgtgcggctctactacatcggaggggaggtctttgcggagtgcctcagtgacagtgctattttcgtccagtctcccaactgcaaccagcgctatggctggcacccggccactgtctgcaagatcccaccaggctgcaacctgaagatcttcaacaaccaggaatttgctgccctcctagctcagtctgtcaaccagggctttgaggctgtctaccagctgacgcgcatgtgcaccatccgtatgagcttcgtcaaaggctggggagcagagtacaggagacagacagtgaccagcaccccctgctggattgagctacacctgaatggacccttgcagtggcttgtcaaggtcctcacccagatgggttccccgagcatccgctgttccagtgtgtcttagagacactaggagtaaagggagcgggttggggagggcgggcttggggaaaatgaccttggaagagaactccatccaacttggtcttgtcaaagaacaccgattccactcaactaaggcaccagcctgtttctgagaccacagaagaaaaccccagggatggatttatgaacagctgtgtctgctacatacacgtgcccctgtctgaaggccaagtgatggcttctgttctggtggcttgaactaacaggtggtgtatcgccacctgactccttgtttaatgacagaggtctgggatgtcacagtccaaaaggaaagtgcctttctccatggctggagtatggagtttacctttggagaagttgtaatggagcatgccctgtcccaccactctcagagagggtgtacctgtcaaactggatggcctacataggtactcccccctacccctaggatgcagagagacgggaacacgccggagggtagagctggggagaacccattcttccttggaaggatccgctgaaggtcagcgtataggtgatgtacagttcctaatatcacatcagtctcagagtgttcacaggaagcagcaagggcactcgtggagtatgtgtcctgggtgaggtggcaccacaccgaatgaatgcatctctgggagctggcaccacaaccctgatgtataggctgtgttggtttatggagacaagttgcatcaatgaattcacctagcataggctctttgaaatgtcctctgtttgataaaaaacaatcctgggtacgtatgttggctggaaaaccacaatggaccctgccactgcttcttgccctgaggtttggaagctgagagttatagaagccaattcaccaggaggtaagacatcccaggctgacatgggcaaatgaaaagggctattaaaatttttttacaccttggaaaattgcaggcttggtgcagagcgctctgtcatcttcaccctgggatgtaggattacctagctatggtaaaggattgccacagcaaactgtgacactgtgtaatgagcactgttcccagcggcaattacagagaaaacgagtgtaaattatttcaactggaaaaaagtccctttcttggctgttttagaacagggtacacaggatcgccacctgcaactggtactcgcttcttggctgctgcttctgttgtgaaaagacgagcccatgtttctgcatggatttcccatggaagtcctgtcctgctacagaggggaagaaagtgtaccctccaatgtgataaatcttctgatgcccccagactcttggagcacatcctggtgcccctcctgcaggagcctgtggcatatttccagctgggcatgctgatcctccttgagacacagatgcctgtgtgagtctccgttgatacaattctgaacccctcaggttctctgaaagggcacagaccatgggcgtgaacattgtgccgtacctgagttggtctgtggactgctgcttcagacacacgagtcctcggaactgcctggctgcctgtcacgcatgctctgagtcagaacacaccaacgctctgctgtggctcctagggaagcattcatggtcctctgttatcagcaggggtttatgtcacttgctgtccggtttcctaggggcttcctgtgccccttccccagctatcctccaggtggctagggacagtctattctgctgcaactggaaagtagagggaaccggcactgctcagagcagatggcggcttctggaggcacacagtgggagtacaccccttcatgttattggccagttgctggagaatgctgtaggagaaaattctaggcaggtctactcttggcatccctgagagtcaaaggcttggagtctaggaaaggtcacaccatgatggagaacacaggtcatttgggtacgtgtaatcaaagtgccctcccaaatcagttctccttttcgtatgaacagcatctctacttttaaagaggagttgaggatcgagaagatgacagtgcagcagtgggtgtggcctgactacatgtgctgttccagccctgggtgcccactgacaccgacccccaggcagaggcctttgtcttcagcactcctgagaagttggctctttaccccttctcctctgctgcccctccttcctgctggttcaggtagccccagccacgtgggttagagtcctgtgctggcctgccatggcagctggctaccttccagaccaactgtagagatacctggcattttgaagaaagcctagactggagagcagggcctctcttgggaaggacacaaggcgggcaaggctgctggcagacttctccactgacctgagtgtgctttttttttcccctaaatgtattgcatcaagcctcagtgcttatggagtgcagtggtcttcatcttccccaacttgcttctcagagctgggatgtattccagagcctgatgtttttattcaaaccacagaaaagttttctttaagtagcctgtgcagtcatgcatgtgcctgagttgtctggagcagaggcaaacatctgacttcattcttagccccaagctgccatttctgagtccttgagaggctgagaaggctctagctttgtactgtattcttactgtgtgactagatgcgtgagcgctttacattagaaggaacctggttagagctcgctcctcctgtctttgtgtggcatttgtgttccattaccggcccctttaagtaacggccttcacagcaccttcccagtgggtagaaagccacacaccaggatgtgggtcaaccatgaagatgtggcattgcagacgggggaacatgtggatgcatggctatcgccctgaacagtccctgcagctacttgtgttaacacagaactgatgtttagcattctgccgctttcgtatttatgtaacaattccttaaagccattcaaatggctaactatttaatttctttaggacagttgtaaaggtctctctcctgaggacaatgacttggaagaactggggcacagccagtcccagacactggtggaggctgcagtgactttttttggctcaagatccacaagcattagagtagactgggccaacaagtcaacaagtggtggcgtgtgcaaacgggctgccctagtcaagcccagtcccttcaacagtatgtctgatgcaccacaggccctccctactggaagtgggaacttcaaatggaaattggagccatcttttatcccagaagcctttgctgctgccagggcaagtgggctggtgtggactcttgtttaggaggctgaggttcttgtcactccttagccagccaggcctttagtgtctttgtaaaaagcatgtatttatagtgttttagatttttctaacttttgtatcttacagcattgtaccccattgttaaagagccgtgtcccctcttcttataaacgcccttttaataaacttttcaccgtaaagctcctgagacaggagcacagtctgt

SEQ ID NO: 36 - protein, 425 aa, Mus musculus
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLVKVLTQMGSPSIRCSSVS

SEQ ID NO: 37 - DNA, 1983 bp, Mus musculus
  CDS (80)..(1171)
tggagctggtcggttatggttggaactttccctgcccttcgttgactgagtgaaccagccagaaatacattcccaggaaatggcctctaaatcttggctgaattttttagtcttcctctgtggatcagcaatagggttttttttatgttctcaactcttgagtattttgttgcgagaagaggctgccattcagcctaacatgcttcacaatgaccctcatgcaaggcattcagatgacaatggacacagtcacctcaaaggacagatgaacttcaatgcagattccagccaacataaagatgagaacatagacgttgctgagaacctctatcagaaagttaaaattctttgttgggttatgacaagtcctcaaaatctagagaaaaaggccaaacatgtcaaagctacgtgggcccagcgttgtaataaagtgttatttatgagttcggaagaaaatcaggacttccctactgtgggattgaaaaccaaagaaggcagagagcaactatattggaaaacaattaaagctttccagtatgtacatgaccattatttagaagatgctgactggtttatgaaagcagatgacgacacatacgtcattgtggacaacctgagatggcttctatcaaagtatgaccctgaacaacccatttactttgggcgaagatttaagccctatgtgaagcagggatacatgagcggaggagcgggctatgtcctaagcaaggaagccttgagaagatttgttaatgcatttaaaacagaaaagtgtacacatagttcctccatcgaagacttagctctgggaaggtgcatggaaattataaatgtagaagctggagattccagagataccattgggaaagaaaccttccatccatttgtaccagaacaccacttaatcaaaggttatctaccaaaaacattttggtactggaattacaactattatcctcccatagagggtcctggatgctgttctgatattgcagtttcttttcactatgttgatgggacaactatgtatgaattagaatacctcgtttatcgtcttcgtccatatggttgtttatatagatatcaacctgccttacctgagaatatactgaaagaaattaatcaagtaaacagaaaggaagatacaaaaataaaattaggcaacccctgaaagcagaacataagtggtctacattgcactgaaggactcttgcctttctacggaaccctgaaatcccagtgaggaactcacctgaagtgaacattccatatagaaatctttcaaatggatgactataaactgaagcatttaaagagctgtgaagtttgctaaaacgtgtcttgatacagtaatatataaatataaataaatatatatgaagaatttgtgtttttaaatggtgacagaagagctagaaagactctgtttctgactgttcccaacagtgtatgttgttatcactacagttggagtggttagtttataatcaccttctgcaccacaccagttagaacacaatgctgaacagatctgccttaaagaaagaaagaaagaactgttgctcagtgttgtttctcagctgatatgtgtatgctgcaaaccagccttccagttgccgaatgaaaactttcctgtgattatattatttataattggcagtgcttcctcaagaagaataagaagggcacttaccccaaagtgcagtaattaaccacatttccaattattccgagccttgactccattagatacttagttctaaagataatcaacccgataccaagtccccattgtcccctttccaaatgtttgaccctgaataccatttttcactattaatgctaatttactcaaagcattggcagcattctgtaaatcatgcattttcaaatatgttgttccccgattcacccattgttaattgtaggtaacaattaaatttaatgctgtttggacaagcaaaaaaaaaaaaaaaaaaaaaa

SEQ ID NO: 38 - protein, 363 aa, Mus musculus
MASKSWLNFLVFLCGSAIGFFLCSQLLSILLREEAAIQPNMLHNDPHARHSDDNGHSHLKGQMNFNADSSQHKDENIDVAENLYQKVKILCWVMTSPQNLEKKAKHVKATWAQRCNKVLFMSSEENQDFPTVGLKTKEGREQLYWKTIKAFQYVHDHYLEDADWFMKADDDTYVIVDNLRWLLSKYDPEQPIYFGRRFKPYVKQGYMSGGAGYVLSKEALRRFVNAFKTEKCTHSSSIEDLALGRCMEIINVEAGDSRDTIGKETFHPFVPEHHLIKGYLPKTFWYWNYNYYPPIEGPGCCSDIAVSFHYVDGTTMYELEYLVYRLRPYGCLYRYQPALPENILKEINQVNRKEDTKIKLGNP

SEQ ID NO: 39 - DNA, 4263 bp, Mus musculus
  CDS (602)..(1576)
ccgagaaggagagagcggcacaggtttctgccgcggcgctcgaacgggagcttagggacgcagcatcctcggtgtccacccgggatcgcagctgcaccggccgacatgtgacggaggaaagccctcagcattcccggcaaccgccttcaaaggggtcccggaacgctcgcccaggccagcagctggatcctcttagccctcagcgacgcgggacaggagcggcaaggtacaacgcctggtacgttccaggctcgcactcgggaaaagagcgcagcggggaccgccgcctggcttgcactcggcacaactccgggacctgcggagtgcggctggaccgtgtccagattcccgccgacttcatccagagtgacccaagaccctagccggtcctcctgcaccccctggagttctagcttgccctgcatctgcgccgaccccacaacgcccaggtccccgcaacgccccagtccggaaggggagcgcgcttgcggcttctccctgattgcagacgctgccgctgtcctaactgcacacagaggtcccctacccggagcctagtccttgcagggggtggtgtccgagacgctggggagcgcgcaccatgaagtccgcgctgtgcagccgcttcttcatcctcctgccctggatcctgattgtcatcatcatgttggacgtggacccccgcaggcccgcgccccaactcacttcccgtccctacttctctccccatgcagtgggctgcgggggctcccgagtcccgctccgacggagtagccccgggcgtgacgctgccgagaagcggaacgagtctcggcctcagctgcagccggagccgcgcttgcccaccatctatgccatcacacccacctacagtcgcccggtgcagaaagccgagctcactcgcctggccaacaccttccgccaggtggcgcagttgcactggatcctggtggaggaccgggcgacgcgcagcgagctggtgagcagcttcctagctcgggccggcctgcccaacacgcacctgcatgtgcccacgccgcggcgctacaagcgaccgtggctgccgcgcgccaccgaacagcgcaacgcgggcctcgcctggctgcgccagaggcatcagcatcagagcgcgcaacccggcgtgctcttcttcgccgacgatgacaacacgtacagtctggagctcttccaggagatgaggaccactcgcaaggtttctgtctggcctgtgggactagttggtgggcgacgctatgaacgtccgttggtaaaaaatggcaaagttgttggctggtacaccggatggagagaagacaggccttttgccattgacatggctgggtttgctgtgagtctgcaagtcatcttgtccaatccgaaagctgtatttaagcgccgtggatcccagccagggatgcaagaatctgactttctaaagcagataacaacagttgaagaactggaaccaaaagctagtaactgcaccaaggttctcgtgtggcacactcggacagagaaggtcaatctagccaatgagccaaagtatcacctggacacagtgaacattgaggtgtagccgccaggaccagcaggtgaggacaggaagtctggagcgtgggctgctgcccattcaggtactgttcctttgccttcagcctttccttaccgccgatggacggccgtttcgacagctacctggatgctacaaaatgtgttgtcttcatttgtctgcatgtgttttctgaactggaagagttttgtagagtgaagccacatgtcactgaaaaggacaacgaagtatctcacactgctccctccctccctccctccctccctccatccctccctcccacaagccatctcaatactgctagaatgacaggttttagaagctgatgaggagtactctgtcaattaaatattcccagttaaattcttggcttcacaacatgaactatttggaaagctgatggcagtgtacactgtaggaaataaaacccacaaataaaaaggtgtgtggattcatctgtttaatatagggaaataacattttgtcaatactaggcaccataaaatgtaaacacaattactgtcataaacctggatataccttcaaggattaaaagtggctttgttttagttaccctgtgtgcatatagtgcagaaaaaggtcttcatattagcactatgtacatgagaagaggcccacattacaagaggcaatggaaatttgaattctgaaaccttcattgagttttacagtagcatccaggtttatctccgttcactaaccacgttccctgttaagcacatcataacaacagcacagtggagtgagtgatgaataaaagaatttgatacactagaaaacagtgctcagtgatacatttacattctatttatatgattaaacatttgatcatacagtacctttctacaggattactggctaatttgggggtggggtttatactgttagaggtattactaacatgataactacttcccttatatgcaaacgttagagctataattttgcaaataaaactgattatgcaagttaactgagcagcttcccaatgtggcttaggacatcatggggagtatgagcaaagctgccctccacacagatggtttctcaacctgctttcacatccaatttaatcagcacagaccaacacggtcaatcaggtcattcttaatgtgagcaaatgggggaaagaaaaaatacgttatgtacatgaataaatgggaactagatgcctttgcagcgaggaatgttggggtcggttctggatggaactctgtgacagcttcttacttccacagctgtgtgctcagcgtctgccccgatgagcttccagaccaccctgctgtcgtgctgggaggctggccaaaacctgcactagcgttagcactactgaggttcatggcagccatctgctgattcatctgcgaaacacaaaaggcttggtttacaaggaggaccctcttttcactcctgactccttagctacagccagacacttcatcagatgtgaaaattagtccagacactggatgtggactacactgaagtctttctactgaaatcagttacttttttcttacttttttctgagttaggtgttacttataaagtaatccataattagttcactttctagattaaccacaactgaaaacacatcaaagtaatctttgagtttatatctgagaccatcactcttctgtgactccaagactgagacattttagaaagtaaaacatctaaaattaccctaacatttacaaaatactcatacctctctattaacatcctaaatgaccttaatatacttcctaaacattaaaaaggtcatatagcatatgttttaggtttatgttataataacataaacagctaaaattaaatttcattatatgctatacaataaacaactacatgtacttttctttcaggctaaaatccagtatatttatcaggctcttagcacagtagttctcccactacccagtttggtgacggaacaagagatgcaggatttacctaaaatagtttcaaacctatgttcaacattatctgtgttctttgtgcaaattttttttaaaacccaagacataataagcacacatgtgtctacagacaggtagcttccatcttcccttctgctcactcttgtggccttgctgttcttcactggctctcctgtactctaacctgagtcatgtgagcctgagcatcctggtcacagaaaagctgttcctagtatctgacatcattcccgaggtttccaatgcataagcagtgtgtacgtatatacctataaaccgcagctgtcacagcacacgagcatttgtaagatacacggtgattctgtgaatgttagttagcagacattgttctgaaggtaaactaggggtcttagtggtcctggttcctgtgagatgctcggtggaccatcatgcaggaatgggaatgggaccatgagcactaagtcaaagcctctgatgtggggtaacagcagaaatgttactctaacctaagccagacagacatggaatttatcagtgaatacctgagggaagcagggtttacattctcaccatggggattcctgagccaatggatgttcctcatacatcaacaggcagtaaccacatgacagcggcagccttgatccctcataaaactgtgctttgaaaatatgaagctggatttctatctcactgataagaattaataaaaaaaaaaaaaaaaaa

SEQ ID NO: 40 - protein, 324 aa, Mus musculus
MKSALCSRFFILLPWILIVIIMLDVDPRRPAPQLTSRPYFSPHAVGCGGSRVPLRRSSPGRDAAEKRNESRPQLQPEPRLPTIYAITPTYSRPVQKAELTRLANTFRQVAQLHWILVEDRATRSELVSSFLARAGLPNTHLHVPTPRRYKRPWLPRATEQRNAGLAWLRQRHQHQSAQPGVLFFADDDNTYSLELFQEMRTTRKVSVWPVGLVGGRRYERPLVKNGKVVGWYTGWREDRPFAIDMAGFAVSLQVILSNPKAVFKRRGSQPGMQESDFLKQITTVEELEPKASNCTKVLVWHTRTEKVNLANEPKYHLDTVNIEV

SEQ ID NO: 41 - DNA, 1599 bp, Mus musculus
  CDS (175)..(1182)
gaagtggtcccaggtgtcgctcagagtcccgcgttcccgggaactagtaagggagcctggggaaggcggggcgcggcccgggcggccggggcggggccggcgggagggcggagggccgggcggacctgtccctgccggtcccggtggaccccgcctgccccggtggcaccggccatgaagctgaagctgaagaacgtgtttcttgcctacttcctggtgtcgatcgccggcctcctctacgctctggtgcagctcggccagccttgcgactgcctccctccgcttcgagctgcagctgagcagcttcggcagaaggacctgaggatatcccagttgcaagctgatctccgtcgcccaccccctgtcccagcccagccccctgaacctgaggccctgcctactatctatgtcattacccccacctacgccaggctggtacaaaaggcagagctggttcggctgtcccagaccctgagcttggtgccccgtctacactggctgctagtggaggacgctgagagccctaccccgctggtctcggggctgttggccgcctctggtctcctctttacacacctggctgtccttacccccaaggctcaacggcttagggaaggtgagccaggctgggtccggccccgaggagtggaacagcgcaataaggccctcgactggctccgaggaaaagggggtgctgttgggggggagaaggatccaccgccaccagggacccaaggagtcgtgtattttgctgacgatgacaacacctacagccgggagctctttaaggagatgcgttggactcgcggtgtctcagtgtggcctgtggggctggtgggtggcctgcgatttgaaggtcctcaggtacaggatggccgcgttgtgggtttccacacagcatgggaacccaacaggccctttcccttggacatggcgggatttgcggttgccctgcccttgctattggctaagcccaatgcccagtttgatgctactgcaccccggggccacctggaaagtagtctcctgagccaccttgtagatcccaaggacctggagccacgggctgccaattgtactcaggtactggtatggcacacccggacagagaaacctaagatgaagcaggaggagcagctacaacggcagggccagggctcagacccagccattgaggtgtgatggcaacctcaccctgacttctaccttattttaggcacataccttgtgggactgggctccaggccagcccaggatgtggttttttttttaaagacctgacccctgagaaccagaggcagcccctccaaccacagggtgtgtcccagttgcttccctcccttcccccagcctgccatgtggcactgcccacatgttggggacaagcagcttctctagtgagccagatcagccccatctggggcggagcaggacagatggactcagaaaggagggtagctgtgggaaaagggtaacttattggggacaagcatgcagtggggggctagaggagctgggctggaccctccccacctgagcatgcgatccccttcctacctctagaataaagaatctcaacccggaaaaaaaaaaaaaaaa

SEQ ID NO: 42 - protein, 335 aa, Mus musculus
MKLKLKNVFLAYFLVSIAGLLYALVQLGQPCDCLPPLRAAAEQLRQKDLRISQLQADLRRPPPVPAQPPEPEALPTIYVITPTYARLVQKAELVRLSQTLSLVPRLHWLLVEDAESPTPLVSGLLAASGLLFTHLAVLTPKAQRLREGEPGWVRPRGVEQRNKALDWLRGKGGAVGGEKDPPPPGTQGVVYFADDDNTYSRELFKEMRWTRGVSVWPVGLVGGLRFEGPQVQDGRVVGFHTAWEPNRPFPLDMAGFAVALPLLLAKPNAQFDATAPRGHLESSLLSHLVDPKDLEPRAANCTQVLVWHTRTEKPKMKQEEQLQRQGQGSDPAIEV

SEQ ID NO: 43 - DNA, 2974 bp, Mus musculus
  CDS (197)..(2203)
gataggggccccaggcttcccacagcatggagacacagggagtgtggagggtgccccccagcccacggacaacaccttcactccgaagtgtgagattgtgggcaaggacgcactgtcagcactggcccgggccagcaccaagcagtgtcaacaggagatcgctaatgtagtgtgcctgcaccaagctgggaacctaatgcccaagtctgtgccccggcactgccagctggctggcaagatgagccctggcgtccaatgggaagagatccgggcccagcagcctgttggtggccctccagtacgcatcgcctacatgctggtggttcacggccgtgctatccgccagctgaagcgtcttctgaaggccgtctaccatgagcaacacttcttttatatacatgtggacaagcgttccaactacctgtaccgggaggtagtggaactggcccagcactacgagaatgtgcgggtgacaccttggcgcatggtcaccatctggggcggggccagccttctgaggatgtatctgaggagcatgaaggaccttctggaaattcctggctggacctgggacttcttcatcaacctgagtgccactgactatccaacgaggacgaatgaggagctggtagcattcttatccaagaaccgggacaagaatttcctcaagtcacatgggcgagacaattccaggttcattaagaagcaaggcctggaccggcttttccatgagtgtgattcccacatgtggcgcctgggtgaacggcagatcccggcaggcattgtggtggatggtggctctgactggtttgtgctgacacgcagctttgtggaatatgtggtgtatacagatgaccccctcgtggcccagcttcgccagttctatacatacacattgcttccggctgagtcattcttccacacagtgctggagaacagcccggcctgtgcgagcctggtggacaacaacctgcgggtcaccaactggaaccgcaagctgggctgcaaatgccagtacaagcacatcgtggactggtgcggctgctcccccaacgacttcaagccacaggacttcctgaggcttcagcaagtctccagacccaccttctttgcccggaagttcgagtcgactgtgaaccaggaagtcctggaaattttggacttccatctatatggcagctacccacccagcacaccagccctcaaagcctactgggagaacatctacgacgtggccgatggccctggcggactcagcgacgtcctactcacagcctacacagcctttgcccgtctcagcctgcgtcatgctgccactgctgtatccccactggccactgcagtctgcaggtttgagcccagggggttgccgtccagcgtgcacctgtatttctatgacgaccatttccagggctacctggtgacgcaggcggtgcagccctcagcccaggggccagcagagacacttgagatgtggctgatgccccagaggtcgctgaagctgttggggcacagtgaccaggccagccggctccagagtctggaggttggtactgagtgggaccccaaagaacgtctcttccggaactttgggggcctgctgggaccactggatgagcctgtagccatgcagcgctgggcccggggccccaacctcacagccactgtggtctggattgaccccacctatgttgtggccacatcgtatgacatcacggtagatgcggataccgaagtcacgcagtacaagcctccactgagcctgccactgcggccgggagcctggactgtccgcttgcttcagttctgggaacccctgggtgagacccgcttcctcgtgctgccgttgacgttcaaccgcaaactacctctcaggaaagatgatgccagctggctgcatgcgggaccaccccacaacgaatacatggagcagagtttccagggcctaagtggcatcctgaatctgcctcaggcagaggccctggaggaggctgcccggaggcacacagagctcacgggttctgcacttgaggcctggacagatggggaactgagcaatttctggtctgtggcaggattgtgtgccataggaccttctgcttgtccctccctggagctctgcagactgaccagctggagctctctgtctcctgaccccaagtcagagctggggcctgtcaaagctgacgggcgactcaggtagcaggaccccagccagcacaacccggaggagccggggaattgcaccttacagacaacggagggacatctctcccacagacaagaaccagaggcccaggcagtttgcccatttgagccaacaggaacctacactgagggcagggaaggtgggtgcagtactccctactgctaacggccctgcaagagaccagcggtccactggagccatgaggaggatcttcctctgctcctcgttagtttatacttttttaataattaaaatgagtggcagagggaggacgatgtgcaatagggctcagagcagccccaggatatgggccatggccatggggagcaccacagagcatatcggtttctgctctggggaggcatgggctgcctccctctccagagcctccatccccagcctgcatctgaactgcgcactgctgaggctctgaggcccggaggacaggtgatctgtgggggctactgtggagttggactttagtgagcaggcctctcctctgggagccagggagccctagagcagagctggggccggctctcgggcttccctaggaaatgcgcgccatcagggcctgctgagatccatgaagtttccctactggaggaggaagcccactggcggcacaggaaggctgtctgtcagagctctcacaagagtcgagttgtgcctgaagctcagtgcgaagtctgaaatatgcaacagaggaaatatatctctatctctccaaaaaaaaaaaaaaaaaaaaaaaaaaaaaaaaaaaaa

SEQ ID NO: 44 - protein, 668 aa, Mus musculus
MPKSVPRHCQLAGKMSPGVQWEEIRAQQPVGGPPVRIAYMLVVHGRAIRQLKRLLKAVYHEQHFFYIHVDKRSNYLYREVVELAQHYENVRVTPWRMVTIWGGASLLRMYLRSMKDLLEIPGWTWDFFINLSATDYPTRTNEELVAFLSKNRDKNFLKSHGRDNSRFIKKQGLDRLFHECDSHMWRLGERQIPAGIVVDGGSDWFVLTRSFVEYVVYTDDPLVAQLRQFYTYTLLPAESFFHTVLENSPACASLVDNNLRVTNWNRKLGCKCQYKHIVDWCGCSPNDFKPQDFLRLQQVSRPTFFARKFESTVNQEVLEILDFHLYGSYPPSTPALKAYWENIYDVADGPGGLSDVLLTAYTAFARLSLRHAATAVSPLATAVCRFEPRGLPSSVHLYFYDDHFQGYLVTQAVQPSAQGPAETLEMWLMPQRSLKLLGHSDQASRLQSLEVGTEWDPKERLFRNFGGLLGPLDEPVAMQRWARGPNLTATVVWIDPTYVVATSYDITVDADTEVTQYKPPLSLPLRPGAWTVRLLQFWEPLGETRFLVLPLTFNRKLPLRKDDASWLHAGPPHNEYMEQSFQGLSGILNLPQAEALEEAARRHTELTGSALEAWTDGELSNFWSVAGLCAIGPSACPSLELCRLTSWSSLSPDPKSELGPVKADGRLR

SEQ ID NO: 45 - DNA, 5532 bp, Mus musculus
  CDS (369)..(1427)
ccagtgcacggcggcggcggcaccttccttctcgcatcccggtcgcccggcccgtgaggaggcagcggcggccgcaggcgccagcagaggaggcggcggagcatcgagcagaggcgaggcggaccggcaggagcgcgcggccggagccacgcatcctcacacttgcacaccaactcctgcgcctttcaccctctagaccagagtcggggcctcggagagcctcggtgaagctagcgggagccagctccggagcccagccagctctgttctgagcctccccggtgctggtacccccgcggcggggtccccgcttctgcgctcgggacgcgcctcccggacagccgggtccccgcggccaggacaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttgaaaagctacatgaagttcctgttcgtgcgggaacccttcgagaggctggtgtctgcctaccgcaacaagttcacgcagaagtacaacacctccttccacaagcgctacggcaccaagatcatccgacgccagcggaagaacgccacgcaggaggccctgcgcaagggggacgatgtcaagttcgaggagttcgtggcctacctcatcgacccccacacccagcgggaggagcccttcaacgagcactggcagacggtctactctctctgccacccgtgccacatccactacgacctcgtgggcaagtatgagacactggaggaggactccaattacgtactgcagctggccggagtgagcggctacctgaagttccccacctatgcaaagtccacccgaactaccgacgagatgaccacggagttcttccagaacatcagcgccgagcaccagacacagctgtacgaagtctacaaactggactttttaatgttcaactactcagtgccaaactacctgaagttggattagagggctgcggaggggagggggaggggtggttgggggagagggagagaatcctgctttttaatttaagatttttatttgtcaaaagaattctatggagactgggttattttgtaagttaatatgtcttcgggggagatgctgcgagcggcatggttaagaattatttaaaaattctccacggggaaggacagctgtctttgcaggggagcggggtggaatatccctgtttttagaagtggatactgcaacactgtctccaaggtgtccttgtgttctggtgaagtccacaaactgcattccataaagtctaatgaatcttatttatagttatttaaacgtggtctgtggcagcagcctgctcctgtctgtgcagaggagagtacagtctgtgcttcgctggctatggctcattgggggaggggagggggttatctcccccactgaccgtgtgtagaatcccatggtgacagctgcgggatgtgtgcctgtggccattcaagaaaccctgctagaaagaagtcaattgtccttgcactaagaaacagggatgagactggttcaaaccgattgctagcaaccccaaagtcccttgtagtaaagaaatgcaaatttttttaacccaagaagagagcagatgatagcgatttctctttaccgaaattagcaggctataagaataggctttctgtggaaatctttaagtggcactttccagtgctagcgaaggcagaataatggttgttttctagtcaataagtattgagcatctactattctccaggcttgactgcagatagcacattgagtgaaaccaggagccttggccttcagcaagctttatgtgaacacagggcatccagctcaacagtgaaccaacacccccatccctttggtcctcacacagactttagactattctcaagtggtccagagggtccaggaccctagcaaatttgcgtatttctacagttcaaatttgaaagccacacatttggatcaggccacttaactagctaagtagttcattgcccaacacaagaaatgacagaaacccttttgatcatgtatatctagtgcagtgtcttcatagtagaagcctataagccacaatattctccaagcgcaaggatggaggactgctgtgattgattagtgatgtctgccttgactaaggaaagggaggaacatttctcaagggcctcctactgtgatctccagtccacatcttgtcctgcctgccacttaaaacagtgcactgcatacatttggagactggcggagttcaagtgtgaaggtcaaaggtggagtgttggtacccacaggcacatgcccaccctccatgtcctgcattggcagagcatccaggacctccctctcctccagaaggttggaaatttgctctttcctgttccttgctgatcaatgcccaaacctattcctcatttctagttgtttctgatactttgcggttgacatttgagccacattcccatgacagccagtgtccgggtacgtagcagatgctgtacatagttctgatgataagggagctggtaggctgtaaattttgctttttgtgtttttgtggggttttgtttgttttgatgcagtatagagtgaagggggtaagaatattctgaaaaacataaaattgagtaatttattcacagaaactattaaaatggtattgttaggctcacaaggaggctgggggcagcggtaactgctacactgtctcgcagaggaaattattttatttaatgtgagagaaatgtgagccagagtggctgcctgaggctatttataaaacgcaggtaccttccccaatttctctcccctcgcctttaataaatagatttatgtcatgtgtgtgaccaagaacagtgacagatcttggctgtccctggaatagagaggcagtcatggtgctcaggccctgggcgtggtgtagctgatgcatgtcccaccgagcccaggctctccaaggcatcagtttgtggtcacatatccttagaaaacaaaattaagtcaggttcctgtctgcaggagttttcagagccttgaatacatagtctgccttgtgaaactgggagaatgtagcatttctgggctgcatgacctaggtatcctgtatggcagaccatgtcttagctggtttttcacacagtagccttcagtgagggtgatctagcctgtctgaggtctgcaaagattgacagctgcttcctagatccttccacaaatcagcagaaacagtgtccatctgggaactttatgagagtgttgctttacctagttggggtgtcttcatctggatactgccccagagagtgatcccgtccctccaagcacagcggagggaagcctagggtgttatggagtggggcaggtggcttaaggacaccttaaaggccacttggtggggataatggcctcgtggggatactcctcacttccacccacagtttggaactgttcactgtgtgcttcaggagttttaaatggagattcgtgttgatttccatggtccgcatctgtttgtcttccgtctgtggtccagtgctgtgtgcagatggtacttactccctttctctctcttacagggagaaagactgtccttctggggagagtgtatcaatacgcttccagaatgagggagtctttctcctgtcttgtgttcacacttcctagtgacaattttcaaaatatgtatcggcttgactgtttattgtagagtgtagaaaggtttctgtgttcctggctaaagacatccattggtgaaatgtgcttggaaatccaagtgttttctactgaggaaaaaaaaatgttggaaaagctttctcctgcatgcctcttgtgcttagctagaaaggggagctgccagcgtcccagctctgagccactgttcaaaggtgcagctgtgttttaggactaggtacaggcagagtgataaggacatgcctgcttagtgtactcatttatcgagaaagtttagactatgagccacaggggtccctgcctgcagtccctatagcctccccctgccccagtgcctttctcagctggtctgggaatcagggcagcccattgaggggggctcagacaaccctttcttcctggttctgggagtaaggctttctctgcagccacaaggagaacatgggaaccagaaacttctctttggtcagcccttctgagcacacaggtttagcccatgctccaagaatgctcctgagcattgtataagcccctaggactcttagctcatgtgataatgattgcattactatcctgctttaactgacaaaaccacagagaattttgcttgctttcctactctcttcctctgcccaggcctcctccatacgtatacatacacacatgggcatgtatacacacacatgcacaatagacatgcacacacacatgcacacatgggtatacatatacacacatgcacaatagacatgcatatatacatatacatgcacacattgataggcatacacacatgcacagtagacatgcacacatgggtatacatagacacacatgcacagacatgcatacacacacatgcacacataaatgtgcacacacacatacacaaattgcccaagcctgtgcactcctggtacccatgtcccatcctggcccacctctttgtcagcaggactgactccatcactccctcaggttttgcccaccagcaccccccagctttcccccagcagagctaaacagcccctctgaatagacaacaggaccttctagaaacagctgaacccttggacagcagcaggcagattttgatctgctctcatcaggtacaaaaggtggtatccgtattctcttctgagcatgctcagtaagcatatggacatagaatttaacatctctgtggagtgtgtgttttttacatatttgtatgcagtcgaggagggcctgttgtagaattctctccctgtatcttactatactgttaaagaagctgaattctatgttgccaacagatgcgtgaaatgttcctccaggaaaagccattcaagcctgattatttttctaagtaacttcaattaaattgaagaagaaaaaaaaaaaaaaa

SEQ ID NO: 46 - protein, 352 aa, Mus musculus
MKPALLEVMRMNRICRMVLATCFGSFILVIFYFQSMLHPVMRRNPFGVDICCRKGSRSPLQELYNPIQLELSNTAILHQMRRDQVTDTCRANSAMSRKRRVLTPNDLKHLVVDEDHELIYCYVPKVACTNWKRLMMVLSGRGKYSDPMEIPANEAHVSANLKTLNQYSIPEINHRLKSYMKFLFVREPFERLVSAYRNKFTQKYNTSFHKRYGTKIIRRQRKNATQEALRKGDDVKFEEFVAYLIDPHTQREEPFNEHWQTVYSLCHPCHIHYDLVGKYETLEEDSNYVLQLAGVSGYLKFPTYAKSTRTTDEMTTEFFQNISAEHQTQLYEVYKLDFLMFNYSVPNYLKLD

SEQ ID NO: 47 - DNA, 1785 bp, Mus musculus
  CDS (149)..(1408)
ggcgatttcggctgcagaatcagcatcaccagcaacagcagcagcggcggtgactgtggcgggcgctaggtccgtctcctagggaccatgtcccagctgtgcacaaggctgaagtgaagggccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaacacgtgcacaacaccagcacgcacctcaccttcaacaagttctggcgccgctacggaaagttctcccgtcacctcatgaaggtgaagctgaagaagtacaccaagttcctgttcgtgcgcgacccctttgtgcgcctcatctcagccttccgcagcaagttcgagctggagaacgaagagttttaccgcaagttcgcggtgcccatgctccgactgtacgccaaccacaccagcctgcccgcctcggtgagtgaggctttcagcgccgggctcaaggtctccttcgccaacttcatccagtacctcctagacccacacacggagaagctggcgcctttcaacgagcactggcgacaggtgtaccgcctctgccacccgtgccagatagactatgacttcgtggggaagctggagacgctcgatgaggacgctgcccagctcctgaggttcctcaaggtagactcccagctccacttcccccccagttatcggaacaggacggccagcagctgggaggaagactggtttgccaacatccccctggcatggaggcaacagctctataaactctacgaggccgactttgttctctttggctaccccaagccagaaaacctgctcagggactgagcccccagaagccctcacgctgcccccaacaaattgaatggctgtcccatgaggccgtcctttgaggatgggaccctgtggcctcctgggttctctcctggcttcctttgcttcctggtgtgacaggcagaggattccacgcccccccctcgcatctggagaccgtggtacagccaagaccgaagcacctcactctccagagttttgcgctccccacccccgcccttttgcaatctggatttgtttactccacagcctgtattcatggaacactgtgttaaatactgttttctaagattaatatatttcagatatatttaatacgaaagtgggaggaagctggagtaaagtgtggcgcccgcaaaaaaaaaaaaaaa

SEQ ID NO: 48 - protein, 419 aa, Mus musculus
MTKPRLFRLWLVLGSALMILLIIVYWDNVGTAHFYLHTSLSRPHILEPLPTQGLVEENVFTSDVDEFLDTLLSSDAKHNDLSRRKTEQPPAPAPSKPVLSHMEENVRGYDWSTHDAHQNPDRDRQQAERRSLLRDFCANASLAFPTKDRSFDDIPNYELNHLIVDDRHGVIYCYVPKVACTNWKRVMIVLSESLLDRGSPYRDPLDIPREHVHNTSTHLTFNKFWRRYGKFSRHLMKVKLKKYTKFLFVRDPFVRLISAFRSKFELENEEFYRKFAVPMLRLYANHTSLPASVSEAFSAGLKVSFANFIQYLLDPHTEKLAPFNEHWRQVYRLCHPCQIDYDFVGKLETLDEDAAQLLRFLKVDSQLHFPPSYRNRTASSWEEDWFANIPLAWRQQLYKLYEADFVLFGYPKPENLLRD

SEQ ID NO: 49 - DNA, 1728 bp, Mus musculus
  CDS (1)..(1116)
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggccgactttgcgccggctgaggtcaactggcggctgcgcgactacctgacctttctcttcgtgcgggagcccttcgagcgcctggcgtcagcctaccgcaacaagctggcgcggccacacagcgcggccttccagcggcgctatggcacacgcatcgtgcgtcgcctacgaccacacgcgcagcccgatgcgctggcccgcggccacgacgtgcgcttcgccgagttcctggcctacctgctcgacccgcgcacgcgccgtcatgagcccttcaacgaacactgggagcgcgcacacgcgctgtgccatccgtgcctagtgcgctatgatgtagtgggcaagtttgagacgatagcagatgatgctgccttcgtgctggacctggtgggtgagcctgggctacgtttccctgctccaccgctgaggccagagaaggaccttacgcgtgagcaggcccggcgccttttccaggacatcagccccttctaccagcgtcgcctctttaacctctataagatggactttctgctcttcaattactctgccccttcctacctgcgactgcaataagggtgttgggtgcaatagagccagtggctgctgtgaccaggaggccaccaggaggtctggaacgaacctggttgtgtggattggagaccttatccagtgggcctgaccaagagtctggccactggtcacactcattccgactgggtagggtacaggttgctttaggtgaccataaccttgtcaggccgtttctgctgttagtttgatgtgtgtctcttcctcccactctgcagatgtcaggcttcttcctaggactccaggtttgtagttctttggtttggtttgagaggccatttctcagtcttgtctgtgtagaacctgtccgtggcatggtgctacaagacagaatgtcatggcttggttcagtgtggcctaaggtcttgtcagcatttactgcttaggagttaacatgagctgcctgccaccccagcagtcacaggatggtgagcactaccactccacatctacctggctgatctacctttgatctcagccttgcaagaggctggatcttcccctgtgtcagcaaaggccaagatgcaatactgtggcagcttttccagctcacttttattttttttgttgtttttttaaataaatatgttttgttac

SEQ ID NO: 50 - protein, 371 aa, Mus musculus
MTVACHACQAQHGKTLLLQAALAGGGKSGCCTPAPVRPASRVTTGKDAQDTEWQGSPKALLGVPTFENKALGSSWFGGVRKSPLQLLRDLDQGPRSAMAEVHQQRRELLRRACSRHTRRQRLLQPEDLRHVLVDDAHRLLYCYVPKVACTNWKRVMLALRGRGDPSAIPAHEAHAPGLLPSLADFAPAEVNWRLRDYLTFLFVREPFERLASAYRNKLARPHSAAFQRRYGTRIVRRLRPHAQPDALARGHDVRFAEFLAYLLDPRTRRHEPFNEHWERAHALCHPCLVRYDVVGKFETIADDAAFVLDLVGEPGLRFPAPPLRPEKDLTREQARRLFQDISPFYQRRLFNLYKMDFLLFNYSAPSYLRLQ

SEQ ID NO: 51 - DNA, 2073 bp, Mus musculus
  CDS (129)..(1259)
ccggccctggtccctgcctgcaccccgggagctggccacccactatccctcccctcccgagacctccagcccctgctgcagtcacctcccctgcagcctcgaggtcggcgaggtctggccgcagcaccatgtttccccgccctctgaccccactggctgccccgaaaagcgcggagaccctgggccgcacgccaaggcgggccccattgggccgggcccgggctgggctcggggggccgcccctgctgctgccgtccatgctgatgttcgctgtaatcgtggcctccagcggactgctgctcatgatcgagcgaggcatcctatcggagatgaaaccccttcccctgcaccctcccagccacaaaggcgcggcctggagcgggacagatcctaagcctagaggcctatccttggatgctggggactcggacttgcaagtgagggaggacatccgaaaccggaccttgagggccgtgtgcggacaaccaggcatgccccgggacccctgggacttgccggtgggacagcggcgcaccctgctgcgccacattctcgtaagtgaccgctaccgcttcctctactgctatgtccccaaagtggcctgctctaactggaaacgtgtgctgaaggtgctggctggcgtcctgaacaacgtggatgtccgcctcaagatggaccaccccagtgacttggtgtttctggcagacctgcggcctgaggagattcgctaccgtctgcagcactacttcaagttcctgtttgtgcgagaccccttggaacgcctcctgtctgcttaccgtaacaagtttggagagatccgagagtaccagcagcgatatggggccgaaattgtcaggcgctacagggctggagctggtcccagccctgcaggggacgatgtcaccttcccagagttcctgagatacctggtggatgaggatcctgaacatatgaatgagcattggatgcctgtgtaccacctgtgccaaccatgtgctgtgcactacgacttcgtgggttcctatgagaggctggaggctgatgccaaccaggtgctggagtgggtgcgggccccaccccatgtccggttcccagctcgccaggcctggtaccggccagccagcccagaaagtctgcattaccacttgtgcaatgttccacgggccctgcttcaagatgtgctacctaagtatatcctggacttctccctctttgcttacccactgcccaatgtcaccaaggaagcctgtcaccaatgacagtaggccagcaccttttggagtttgggtttaatgatatcagctttgggatgtctttcagagaaactcctggctctgggtggcttcctggtttctctaggtgtctccatatctcagtggtaaggactgtccttggaggtccttgtccacagtggctcagaggacagagctagaaaggaggcctgctgctttcactggtgaactgcctctcttaggggcctgtggtatccgtgtctgcagggcaccagtggttattaaagccatatgtttgatcgaaagactgacttcagccccctggctgctgggtctatgcagtccacctggtctgttgtaatttaacctgtggccaaatcccaaatatgacactagccaagcacatgatcatgcctaggaccaatggctgtgaccccctattcacccatcccatggacctcaggactggagtgagctgtggtgccttagaaatgaaatgtgtgcaattctactccagacttttacatttcctcctcttgctaggtctgaatcatttttctaaggaaagagaaacggaagtggggcccttacctcgaagctctaaagcccagcccctcaagcatccaaagacgcctgtgcctgacctcttcctagggctcctggagcatcttcaataagcctcccttccctacaaacctttggagactatgtgagactgtatggcccatatatctggctgtcacttgtctaatgcatttatttaaaatgtgtatattttaataggatccttgtaagggctgacttttaataaagctttttcatatacaaaaaaaaaaaaaaa

SEQ ID NO: 52 - protein, 376 aa, Mus musculus
MFPRPLTPLAAPKSAETLGRTPRRAPLGRARAGLGGPPLLLPSMLMFAVIVASSGLLLMIERGILSEMKPLPLHPPSHKGAAWSGTDPKPRGLSLDAGDSDLQVREDIRNRTLRAVCGQPGMPRDPWDLPVGQRRTLLRHILVSDRYRFLYCYVPKVACSNWKRVLKVLAGVLNNVDVRLKMDHPSDLVFLADLRPEEIRYRLQHYFKFLFVRDPLERLLSAYRNKFGEIREYQQRYGAEIVRRYRAGAGPSPAGDDVTFPEFLRYLVDEDPEHMNEHWMPVYHLCQPCAVHYDFVGSYERLEADANQVLEWVRAPPHVRFPARQAWYRPASPESLHYHLCNVPRALLQDVLPKYILDFSLFAYPLPNVTKEACHQ

SEQ ID NO: 53 - DNA, 6000 bp, Mus musculus
  CDS (496)..(1914)
cttagccactgaaccatctctccagcctctgttttggggtgtttgtttgagacagaatcaaaaggctggtctaaagtacatattgtagctaaagatggcttccaactcctgccttgcctccagagtgttagggtgtgacctttaaatgacctattaaagaaaccccaacaggtgtagagtacacctgtgatcccagctcttaggaggttggacagatggataaagaggtccaggtgtcccggactacatagagagaccctatctcaaaagagaggcgaagacttaggttttgagagtgggcctgaggtctctcttcaaaagttttatagaaagatgtctctgttccctgtctatgaacacggatggcctgagcacctgtctcttcacaggatcagagtgtcccccacctgaagagggctgattgggtcccaagctatgctcctgagctgagtgcctgcagccagtctgaggaactccatggcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtatgtgctggagcccttcatcagccctccgcccgaggaccacttgactcagttcctgttccgccggggatccagccgttcactctgcgaggatccggtgtgcacacccttcgtcaagaaggtctttgagaagtaccactgcaggaaccgtcgctgcgggccactcaacgtgaccttggcgggcgaggcctgccgccgcaaggaccacgtggccctcaaggctgtgcgcatccgtcagctggagttcctgcagccgctagttgaggacccgaggttggatctacgagtcattcagctggtgcgcgacccccgggccgtgctggcttcacgcatagtggcctttgcgggcaagtatgagaactggaagaagtggctgtccgaggggcaggaccagctgagcgaggatgaggtgcagcgattgcggggcaactgtgagagcatccgcctgtctgcagagctgggcttgcggcagccagcctggctgcgcggtcgttacatgctggtgcgctatgaggatgtggcacgcaggccactgcagaaggcccgagagatgtacagctttgcgggcatccccttgaccccgcaggtggaggactggatccagaagaacacgcaggcgacacgcgacagcagcgatgtctactccactcagaaaaactcttctgagcagtttgagaagtggcgcttcagcatgcctttcaagctggcacaggtggtacaggctgcctgtggcccgaccatgcacctctttggctacaagttggccagggatgccgcctcactcaccaaccgctccatcagcctgctggaggagcggggcaccttctgggtcacgtagtgggggatgtctgggacccttggaactccttcttgtgaaaggctggccctgcttccctcacacccagcctggcagtgagacatagccctggcagaaagtcaaaatggggagcgatgatgggaacatagcccctggctgtagcggtagggccccctgccagctagactccccagagcagctacagctagggccttgggctcctctgaggacaccctgtctccttagtggtactggtcataaggtgtcctcaccctatgacctgcagtgtcccgagcaggtcaaggtaggttcctgtgtgtggacacacctccctagctctttctcacacagtctacacgaagcctgtaaaggcctgtaggttgtgtgggctggagtccaagtatttaacaaccagaaggggtgtagaccctctgcagcctctcagacctcctactgtattaagtgttaacctcttccgctctgagtcagagaagctgggatctggctgagtcctgggaaggaggaggacagcctagggtgaggaaggggacctttgaagctcctcagggaacagtggctgtgtaccagctcatagaaaatggtttgatcagctgttctagtcactcatgagtatcaatcagcctgtgtagagcaagacacacaaagtgcattgaaaacagacaaggccaggggtgtggctcagcggtaaagcccttgcctctctcatgcccagggccctaggtgtgattcttagcactataaatataagggaaaataagccatacatacgtgtgcagtacagagagagacagaaaagcgtaataaacattcccccctctcataagcacacgctctgcctctgtctctgtctctgtctctctctgtctctgtctctctctgtctctctctctcactctctctctctctctctctctctcacacacacacacacacacacacacacaaacacacacacacacactggtacagaaaagcttcccttctccactctttatgctcaccatgtttttaggatttttgtttgtttgttttgttttgggtttctatatatctaaacaaggagccttaaattacatctttttgggattacgtggcgagggggagcagcagggagcatttgcttctggtaaaaggatagaccagaaaggacttccccttggatgatcggagtcatgcagagcccccagggtcccacatgttctgtgtgaacttcatgtggaatgactcacagaagtgactaccttaggcgctcgctctgtagctcaatgtgcgtagagtttgtctattgtataggaagccctgggttctgtgccctgccccacaccatataggtatgatggcgtgtacccatatcccagcattcaggaaatcaaaggttggcggtcaaaagttcaaggtgcagctatgtatcttttgagatgtccgagtctataagctccctctactgagagctgctagtattccttctcaagaacaggttatttgaaaatctcttcttgtaagccccaacatttgggggaggggaggatgggggaactcttgactgagcagctaggtttccccaagcagaagttcttctctcccagtgcaagctgaggtttagtgccccggctagcctcggaactggctcagtgggggttggcacgctgacttcctggacaattgctgcatctgacactgtgagcgcccaggacaccggatgtgtagcatggatgtgggcctagcacaggtgtggaagaggacagtcaggacactcactgttggtgtttgttatccacctgttcagccgccgggtcccacacataggtcatctttcgtcactcaagccggcctctgtttttgattttaacaatccaagagcaagtgtgtaggggacaaagagacaccagctcttcatacttgatggagaccgggacaggatgctgcaggcaggctcgggagtgcattatcgtttcttctgagctcttcatccaggccatattcccttttctgcatcgcatctggggtgggaggggctgagctgggagtttccgtccttcttccctgtgggggtgggagatggggctcaaggtctccatctctcggctccctgaggacagaacccccacagtggacctttgggccttctcaggacattgacaatgttgtgtgcactgcaagttgactttatttattttgtaggaaaaagagatggtattctttagcaggatctgaaactgtaccctagtcaagaagtacaattaataacattattattaataacaattgggatgattcaaaggtcacacacacaccagaccacccaagaaccatgtaggagatgaggcggagccaggtcatggaagcccagaatgctacagagattctgtgggtctattattgatcagaaaatacaacatgggggggccttttctatggcataactcaggtgtctgcaaagacagagcagctcaggtgggtggataggcaggcagatggagccttgaatcacatcccctgaggctcagccagccagtgcgaatcctcaaggttcggaaagggacagtgtagcggcaaactactgggcatcttctctgatgcttagaggctcttaccaaggagcttgatggagaaagtggccatgttggtggcatgggacaactgctcacccaacttcaaagatcaaagggcacccaggtggcctatgacagtgacactctttcctaccattaggggtgtctgcccccactgaaagccatgaaatttctggccctaagggggtaggaaggacttaggagtagcagatggtttgatcccatcccccccacacacacacacacatccctcagctgtcttccacattagagccacttcagttgtccatggacttgtccctttgagacatcctggattttgaaggatagaaacatccccaagatggtctcgtgttaatcccacaacagaggcagaagggtcatactgagagagagagagagagagagagagagagagagaggagtgggggccagaccctcaagaagccaagtgggtctggtcaacctgtgcacatgagaaggagggaacatcactaaaatcagggcctgggctggtgtgttgttggtgagatcccgtggagtggctggctagatatggatgagttttctgagcatgctcacacacccccaacttcacattcttagaaatagcacaaccataatgccttacctcaaaaggatgaggcaaagcttgcaattaattcccagtgtctgaagacctccgggatcctttgagctgtgtgtccttgtcagtacatggggacaggctccttaggtattcctgacatagaggtaaggtgctgcccttgctgccttgcataagctgtgacaagcttcttactgggcacatagaacggctctgtcatctgcttccactaaatttgggcttggactttgcctcctgccaaatctccatctctgctggatagtctagtccctagggtctaaccaccaccctccacttcctggtgggtcctacaagcgctgtctttgtgccagtacccggatggtgtcctgcctcatggctaaatggtacaggacatcttcccagactgagtggtcatggcatgtgcatgtatgttcatgagtacatgcatggtgtgtgggggtggatgggtgcattctgttctgcttctggtgcagctacacagccacaacctctttctgtcattgaccttcttggtcttcttgtagccacagataatcttccaatgcccgattctgctgttctcatctgagagctgacaacccagcgctcagagtagagttcatgacctaggaaacccttctcctggtagcttactgaacttatttaattaaaaacgaacatataggggttggagagatggttcattggttaagagcaccggctgcttttgcaaaggacctgagttcaaatcccagcaaccatatggtggcttacaaccatctgtaatgagttctggtgccctcttctggcatgcaggtgtacatgcagatagagcactcctatgcataaaataaataaatcttttaaataaataaataaatgaacatgaaaaaaaaaaaaaaa

SEQ ID NO: 54 - protein, 472 aa, Mus musculus
MEKGLALPQDFRDLVHSLKIRGRYVLFLAFVVIVFIFIEKENKIISRVSDKLKQIPHFVADANSTDPALLLSENASLLSLSELDSTFSHLRSRLHNLSLQLGVEPAMESQEAGAEKPSQQAGAGTRRHVLLMATTRTGSSFVGEFFNQQGNIFYLFEPLWHIERTVFFQQRGASAAGSALVYRDVLKQLLLCDLYVLEPFISPPPEDHLTQFLFRRGSSRSLCEDPVCTPFVKKVFEKYHCRNRRCGPLNVTLAGEACRRKDHVALKAVRIRQLEFLQPLVEDPRLDLRVIQLVRDPRAVLASRIVAFAGKYENWKKWLSEGQDQLSEDEVQRLRGNCESIRLSAELGLRQPAWLRGRYMLVRYEDVARRPLQKAREMYSFAGIPLTPQVEDWIQKNTQATRDSSDVYSTQKNSSEQFEKWRFSMPFKLAQVVQAACGPTMHLFGYKLARDAASLTNRSISLLEERGTFWVT

SEQ ID NO: 55 - DNA, 2080 bp, Mus musculus
  CDS (147)..(1601)
caccgcccggctgctccactccgctccacccaacattgagggagacccgaagaggccggagccgaggactttgggctgggttttctggacagaaccagcaggcgcctactctgctctgggtggggagagtgaggattcggtgaactatgaagggccggcggcggcggcgccgaaagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgaccccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgctccgcgccctggaggccgagtgccgcaagtacccggtggtggtcatcaaagacgtgcggctactggacctgggagtgctggtccctctgctgcgtgacccaggcctcaacctaaaggtggtgcaactcttccgagaccctcgggccgtgcacaactcgcgcctcaagtcgaggcagggactgctgcgcgaaagcatccaggtgctgcgcacgcgccagaggggcgaccacttccaccgggtgctgctggcgcatggagtggatgcccgtccgggaggccaggcccgggctctgccctcggcgccacgcgctgatttcttcttaaccagcgcgcttgaggtgatctgtgaagcgtggcttcgcgacctgctattcacccgcggcgcgcccgcctggctgaggcgtcgctacctgcggctgcgttatgaggacctggtgtggcagccccaagcccagctgcgccgcctgctgcgcttctctgggttgcggacactcgccgcgcttgatgccttcgcattcaatatgacgcggggctcggcctacggcgccgatcgtcccttccacttgtctgcgcgggacgcccgagaggctgtgcacgcctggcgcgaacgtctgagccaagagcaggtgcgccaagtggaaaccgcctgcgcccctgccatgcgtctgcttgcctaccctcgaagtggggacgaacgcgacaggaagaccgtcagggaaggggagacaccactggagaccaaggccaattgggctgtgtaataccctgatccctgaaccctgccccggggcgtattcaggtcagtggccataaaaaggtgaactcagcatgctgcccccgcactggagaggctgcacggtggaggcgatctatcacactgtgagacactgggactgatttggtatcaactgctgtgccattctcctggtcaggagcatcacaagctgttaagtaatgacagacaccttggctgagatgaagtttccagaaaggaagtaacagtgcaatgtggatatttgtgaccacaacataggaaaagctgtacttcccaggctgaacttggctcagcttgagccatttcaacaaggcatcctcacaataatgaagagatgtgatctggtttcctttcacatcagccaagatgtctggacaaaaccatcaatgtgaataagggccaagtgcagttgtgtctctcttgattaaattacttcatattaaataaaaaaaaaaaaaaaaaaaa

SEQ ID NO: 56 - protein, 484 aa, Mus musculus
MKGRRRRRRKYCKFTLLLALYTLLLLLVPSVLDSHSEQDKGRNCPGLQRSLGVWSLEAAAAGEREQGAEVRSLAEGNPDRSPGSPGNLSAVGEAVTQEKQHIYVHATWRTGSSFLGELFNQHPDVFYLYDPMWHLWQALYPGDAESLQGALRDMLRSLFRCDFSVLRLYAQPGDPGERAPDSANLTTAMLFRWRTNKVICSPPLCPAAPRARADVGLVEDKACESTCPPVSLRALEAECRKYPVVVIKDVRLLDLGVLVPLLRDPGLNLKVVQLFRDPRAVHNSRLKSRQGLLRESIQVLRTRQRGDHFHRVLLAHGVDARPGGQARALPSAPRADFFLTSALEVICEAWLRDLLFTRGAPAWLRRRYLRLRYEDLVWQPQAQLRRLLRFSGLRTLAALDAFAFNMTRGSAYGADRPFHLSARDAREAVHAWRERLSQEQVRQVETACAPAMRLLAYPRSGDERDRKTVREGETPLETKANWAV

SEQ ID NO: 57 - DNA, 512 bp, Mus musculus
atgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatat

SEQ ID NO: 58 - DNA, 536 bp, Mus musculus
acaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttg

SEQ ID NO: 59 - DNA, 660 bp, Mus musculus
gccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaaca

SEQ ID NO: 60 - DNA, 600 bp, Mus musculus
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagatgtttggcagctgtgagctatgggtagtcagtggggagcggcagaaagtgggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggcc

SEQ ID NO: 61 - DNA, 600 bp, Mus musculus
gcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtat

SEQ ID NO: 62 - DNA, 720 bp, Mus musculus
tggggagagtgaggattcggtgaactatgaagggccggcggcggcggcgccgagagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgagcccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgc

SEQ ID NO: 63 - DNA, 600 bp, Mus musculus
accccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtg

SEQ ID NO: 64 - DNA, 735 bp, Mus musculus
atatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttg

SEQ ID NO: 65 - DNA, 574 bp, Mus musculus
ccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccact

SEQ ID NO: 66 - DNA, 1430 bp, Mus sp.
tgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaata

SEQ ID NO: 67 - DNA, 21 bp, Artificial
  An artificially synthesized primer sequence
gacccagatcatgtttgagac

SEQ ID NO: 68 - DNA, 21 bp, Artificial
  An artificially synthesized primer sequence
atgcctgggtacatggtggta

SEQ ID NO: 69 - DNA, 20 bp, Artificial
  An artificially synthesized primer sequence
acgtgccttttacacccaag

SEQ ID NO: 70 - DNA, 20 bp, Artificial
  An artificially synthesized primer sequence
gtgtgcccttttctgtggat

SEQ ID NO: 71 - DNA, 25 bp, Mus musculus
acccccaactcggaacgatgcggct

SEQ ID NO: 72 - DNA, 24 bp, Mus musculus
tgcatgttctcgtccatcctgctg

SEQ ID NO: 73 - DNA, 26 bp, Mus musculus
cgccaccgtgtactgtactgtgaagt

SEQ ID NO: 74 - DNA, 25 bp, Mus musculus
aggctgctccaactggaagagggtg

SEQ ID NO: 75 - DNA, 24 bp, Mus musculus
atatagtatctaggatatatgtag

SEQ ID NO: 76 - DNA, 25 bp, Mus musculus
gaagtaccaaaagctggctgctcta

SEQ ID NO: 77 - DNA, 26 bp, Mus musculus
ttctatcacttggactatttgatgtt

SEQ ID NO: 78 - DNA, 25 bp, Mus musculus
tacacaactccacatttgtaatttg

SEQ ID NO: 79 - DNA, 24 bp, Mus musculus
ccagaagccaagctcattgttatg

SEQ ID NO: 80 - DNA, 25 bp, Mus musculus
ctgtggagaggttgtactcagacta

SEQ ID NO: 81 - DNA, 26 bp, Mus musculus
atttgcctggaagacaacgtgagagc

SEQ ID NO: 82 - DNA, 27 bp, Mus musculus
gtcccttctgcagaagctgggcccact

SEQ ID NO: 83 - DNA, 9120 bp, Mus musculus
tgtatatctgagtatctgtgtatgtgcatatatgtgtgtctatgtgtatgtgtgggtttgtgcatgtgtgtctgtgtgtacatgtctgtgtgtctctgtgtgtgtctgtgtatgtgtgtttacatgtgtgtgtgtgtttctgtgtggacgcatgtgtatgtgtctccgtgtgcctgcatgtgtgtttgtgtatgtgtgtaggcatgagtacatcatggtgtacatgtagaaattgagagagactcttgggtgtggttctcatctcccaccatatctgaagtaaatgtcttattcacaccactgtgcacatcagaaagctagccttgacctccaaggaactctgtctctgtctcctgtcttattgtaggtacctggggtttataagcctatgctgctgcatatggcttcacttgggttctggggatttgaacttaggtcctcacagttttagggcaagcattttattcactgaacaatctcccatgatccctagagtttattgtaggcaacttaacctttagtcgtcattgagatttagcattgatttcagttccagttgtattctgatgttgagcgctacagcagaggtacccagggactgctgattttctgtggccatgaatcctctagtgattaataaatttcatcataatgttctgtttatttctaaatgtccagacaactctaatttcaaattaatctcatcctaaacagcacaggataaaagttacagattgtcctatcttcattcatgtgatgcctcaaatcagatgtttaatcctttagtcagttatttgtgaaacggaacctaattacacacactcgcatgcgcgtgcgcgcacacacacacacacacacacacacacacacacacacacacacttgtagatttgcccacacagctgcttaagcaatttggagcaaatgtagaaatattatgaaatttcacataaaaatgggttttcctctgtcttcttgagaaatgtgtgagtttcaatatgacagtaatcttctggtgttggactataaccacttgaacccctgaggtactaagtgtgtacctactacgtgcctggattccatcatttccctacttatataagcttctccatcctgtagatcagcactgtgtaatggagtaacacaagtcattgaatcatgcaatcatttaatcaatttaattggcacttctgataagagaagtgggcacatatttagttgtgtttgcagcctgtgctatgtcatttttctctatatatactttaagatttttttcttttttccatttacggggggggggttattccatttaatttacatgcttaatgacatataatttgcatatcaggtgttcctttctttagagttaacttcttttaggaccattgtttataatggatgtatatcatccctaaattaaatcagccattgtctacttaacacaccagtttctctgatgtccctctggctcttcctcctctgaggttctagttataggaaagtctggcattttgatgtttttccatggcttcgtgggctctgatcatccacttacagtgtactttcatctcttgtctacaaactgcatatcatctattgatctgcgttcaaggctactacattttcatgcaattccactttgctgttaaaccttttctatgatccgtttttatacttataaacaatccaagtgttgtactctttgggtataaatgttctgtatttcacaggtcttaacttcttttcagactatacaaagaaagattttctattttttttatttgttgtatgcattttatgcatttagcactgggatttccataacaggcagttaagatcattgaatacatactttgtgtggtaaatataatacggcttatttgattctcccccttaaaatgctcccatcttctcagtcatgtcaggttgctatggagtccaccctgagcaccatgagtgtgatgctgtacagacactcctgatatgccttctgttatttgaactccacctgcaagttttcattcaagcatagctcctaatcccattcagtgtgcacttttcttaattgatttgctggtagagagctatatgtaagcatggcttagcatcaaattgaactttctcctctccctagcttctttccagtatattcttcctcaccttcatcagattttgaggtcttgagatctaaaccttcatgttacaatttgcagagataccaggatctctatcagacttgatgtgtctccatgtaattctagtatcagcctttcataagataattgcaaaatatagaaccccccctctctccttttgactattacagctttgtttctctcccaagttaatacagatgtatgcaaatcaactaagaaaagcaaacactgaatgggataaggagctatactcaaaagaaagcttgcagttggagttcaaataacacaagtggaaccaagagtatctgcacagcatcagactcactgtgctcaggatggaatccatagcaacctcccataactgagaggatgtgagccccttttaaagggaaaatcaaataagcagtattatattaaccacatgatgtgtatattcaatgtagattgggctgcaccgaagttatgattttagtaatgagtatttctccttgggttttcttctggaccaccctggttttccttttaaaaatagtaaaatagcagcaatgactgcttctcttaaaatctgaacaacacagagcacttcagcatcctttcaacctatctaaatccattgtagacctgcactgacttgctgtgagctattgtgtcacaactattattgtagctaaacaccttgaattttcaaagctgctctggactgtgtcttcttttagaattaaacttgctaaagatattggactctgagatgcttattcaccactttctataaaagagtaaggatgacaatgctttgagtagaagaagattgcatacgagggtaccagcatgtcattatgtccccagcagtcagtaaagtaaggatccagtgatggtagagttattatgtccattatggaactaatgtaaatgtttatcaatgactagaatggctgtaatcaccaatccttagacaagtgtgagtaacatcatgctcttttcttataataatagatagtcacacacaccatggtcttaaacattacattttgatttttctctttgtgtctgtcttttcgtacctctactccacttacctgagccttcagctagggctcctccaagacggcagtcacagtactagtgaggataattctcatatggagacctgaagcagaataaccttcttccaaggaaaccaaaatcctagtcaacatcttctccactctttggttatttatttttatttctatttctattttgccatttaagaatttttaaactcttttctgttttcttatatatattcttttttttacaacttttgatattctttatcaaatgatttatcccattgcctttcctcctgcagctatctgtgagttttagcatggatatttagaatacaattggaaattatgaaggtcgaataaaagttctctaagagccaggacctcactagcctagttagttgcttagtttctaatataaggcatggtttatctcctactgtatggcccttaaatccactagacatctgttggcttccaacagcatacaattgctgctattgtatgtttaggtatatatttttatgaaacactatattctgattggtgttcattcttcctatgaaggggaagattttattgcctgctcaaattcaagtcagtcctttttgagtagacaaaggttgtccaatcttgctaaacccttttcttttttgcttggggaatttctttagggaaaattctcaaccatgaaccaagcctagagattgataatagctgaaggcagagtagccagtcttagtaagggatgagagaaagactgaccaaagactgaataggaaatggttgatagcattcattgggactagacaataaagttagctaccccaacatgggcaaatggagaaaatggggagagtcatatggatatgtagggaggagaagaaaggaaggggtgagtttgaagtatggaagagcctttaaagcatttgtaagatttgtaagaaaaagcttttgagagacagagtattacctgccttcaaatcccttcaattactgtccctacaagacatacctgtgctcagctgctcttggaccacaagggggcctaagagcatgtttgtgtccctgtctttatacaggcttctcatctgtatttctcaaacgtaaatacaaatgggggagataacaaatgatatttctaagtggtacacaggtatgagaactgcacacaatctatttatatgtataatggccagagctctcagcagggaacagaggaagcctaggtaactcctgctccagagcatgtctgtatggagacctcagttcagcaactatgaaatctgtgttcaaagggtcatcactgggaacagtttctggaatagcagacatattcattctctctctctctctctctctctctctctctctctctctctctctctctctctctctctgtctgtctctgtctctctctgtctgtctgtctgtctctcccttctgtcaggggtatgcatacatgggctgggaattctatcttccctgcctgctgtctttggtgtctccctaatgtggtgccatgtttacttaacctgtctgaaatggaggataacctctggtctgataactgtcaagttactgaaatgtcagggaattgtgaatgaacccctctgaatgctgggcagatgttttgagcacgtgggtatccatagatttttttttcagcaggatttccttctttaatatattgtgggaacgaaacattgaagtttccaacactaagaaaagcaatgctctgggtttgcaaagaagtctccaaagctgtgggagaggcaatggctttgcctcacaaaggtgatcctgatttggattgggtgttcctcagtctccctcatcctttgtttgtggaccaaacctctgatcaaataatcagtatacagacaagtactgactgactcctgtcactgtttcccatgtaaattctcaaccatgtgtgtcagaattgtttgagtcatcaaagttccgtgatgctcaggatggtgaataccagaaactgaagtctgaaatccaatttggcctttctcgggcactgtaaactgagtgcaaatgtgcccagttccctcgtggcccatttatagcacaataacacagtgcttgcttacaaaatctagtcccgattaaagtatttatttaaagttgctttagacaaatatatttctaaatggaaggatgaaacagaaagaatattccaactgtacagtctatattttgtttctaaaaatttttattagaattctgacttttagaaaacaaatagatattcaaaaactacttctttaattaaataataacatcattatagacattctccagccatttagaagtagcatgattacaaagcattcatgttccctttgttttttgaaacagaagtatttatatgacaaataaaaagcaaaaatctcatatatgtgatagctatagatgcattgataggaattaaatttaatcaaaaagaacttttaatggcaataatgtggctactaacttcattgtttgtaataatgctgcaaatgtttaatgaaaactgttactaacaatacaggagaagattacagatatgattaggtcgtgttgatagaaatggaaagagaaaggtcactcactacctcagccacagaacttgaggaatgtgtgagggtttttgaggaaaaaagagaacaatgtaacatcacagcaacatcttaatctgaaagtagacgacaaaggtttcagagtaggggatcagaaatttaagtatgcagagaagcaaggcatatacggcttagacaatgtatgaagaacagcagcagacagcaagtgaaagaaatcacagtccacatggaaatcacacagaccacagtgtgcacacagagacagaaatcagcaagggacacacggcttccagttatgaaagttagacagcaaaatttaagttgtcccacctaaaacatgatgtatcgtctctcaaatggggaaggggaaggagcagcaaacatctggatatgtctaaaacctcacagagaacatctgggatctttgcatttctccctctgactttctcaatatgttagctacttctagatagtttcaataatggtctctaattgactatggagagacaggtctcatgtatgtgaacatccttaaccaggagatggaatgtgtttccttgtcactcattttctaaggaagtgaattccttctgcattgcccaggattggatgttacgtgaaactaaaaatcattgattaaaaagaaatgtcattaccacagttgggttgaatcaatcaagacttatcctagttacaggcatcacctccccttaagcacaaatactgagaacagctaagctcactttgaaatctgggatgaggaggctttaggtcaagcatctgacaatgtgtgtgtgtatgtgtgtgtgtgtgtgtgtgtgtagtatgattatatgtatatatgtagatgtgtgtatatatttatatgtatatatgtatatgtgagtatgtatatatgtatatgtgtgtatgtgtatgagtgtgtgtatgtatgactatgagtgtgtatgtattgtgcaagtgtgtgtatatatgtgtgtatatgtttatgtatatgtgtattgtatgggtgtgaatgtattatatatgtgtgtaggtacatatgtgtatatgtttatgcgtgtatatgtgtatgcattatatgtatgtgtgtatgtgtgtatgttttcatgtgtatgtagcatatgtatatgcatgtgtgtgtctgtgtgtgtaagcatgtatttactaagaaaaaatcagtttctctagctaatgagtcattgaagtgaagaaaccttacgctatcacattactatgtttccagcataaattgcaatatcagtaagacattccccatcataacttaatggacacttagattaggtcatgcagcagtaaattatgaaagcgtctatgctgagctgaggtgggaaatcgtctttcctgcctccttcctcctgttctttctcctaaaggccctcatcctctttctccccttcctttcttttaaaatattctatcattgatccaagctaactaacccttcccttgctcacatgtgacctgcccagaggaatctatttatagtcaacaataacaggccaaggacatgcgaacaaatgactttacttagccccacttggtgaatctattgggtgaataggtaaggggttatttataggagcttgactgactggcacagctgtatctctgaaaagtcccccaaagcatcatgggtgggaactaaggaaagccggaaatgctggagctggaatcccaacctgcagtggctacatgatgaatgtgtctcctttcctactaaatatttaagactctgggcaggctggttagtcttgtaaaagtcttgtgggtttctggagtcctgggaggctcactgtccctgcagaagcttgttattattgtatatgaaagaaagtgctcaacactgagtcacaaaagaagcccccgagaatcggtggcagaggtggaaatagaagaatccgatgcagttcccgaggcaagagttgatcctctccatggtgacaagaaaacaagagccacagaagcctgaaggctaagactatgacctgtgaggttctcctctcctaggacttgaatgcacttaactctgagggctagcagggcagagattcaggagcgttctcagaggttgtcccttctccttgtatgattctaggagaagctcagtgcataggcagaagttgcaaactctaacacattaacccacatcctgaattgttttatatcaattgttttataactacatgttttaacttttgttttggtttttgaattttgtttgtttgtttgttttctatttttcatatgtgtgcatgtgagccagaagacaggctaagggactctgttctctctttccagcatgtgggtttcagggatcaaagttaggtcccaaggcttcactgggcaacgggacccttagcctctgagtcatcttcttaatctttttctttgaaactttttcattacattgtgtcagtgcacacacacacacacacacacatgcatatacatccacatgcatatacatccacatgccatatgtcatggcacatgtgtggaaagaaatgggtgccttgaggaaatcagttttcttcttccatcacgtggatcttatgacttcaggtaacccatctctgctccccaagcatggctacaagcatttacctgccaagtcatctccctggctcccacattaatttataataatacaccctttcagcctagagagatgactcagtggttaagaactctggctgtgcttcagaaggttctgagttcaattcccagcagctataaggtggctcacaactatctctaatgttgttttctgtcctcttctggcatgcacttgtatgtgcagatagagtacttatatgcataaataaatataaatacattaataatcttttaaaaagaaggaaagattaaaagaacaatacaccctttcttttaagtaagaaaacatgcttagtatatagaataaatgctggtgttgaaaggtgaaattcagaagaaattataggttggaaatatttgaactcttttgtgtcaaaccaaatggtaaagatgatgtgataataaggtaaggttttcttcctacacatatacttgtttaataaccagtataagactatgagtgaacataactaaaaatacatgaaaaaaaactattttaaaaaaacatcactttcagatgactctggtgaaactcactgataaccagcaatctctttgatagaccagagtgtccagtctgaagttaattcttttgctttattctcaaacaatgttgcagaagtatgaaccatagtatcaagatacactgtccacatatgcacctggcctgacttgtgt